SEQUENCE LISTING (35 sequences)

SEQ ID NO: 1 - DNA, 12 bp, Artificial Sequence
  folC - E. coli
ataccatgatta

SEQ ID NO: 2 - DNA, 12 bp, Artificial Sequence
  rpsD - E. coli
agaaaatggcaa

SEQ ID NO: 3 - DNA, 12 bp, Artificial Sequence
  rpsD - K. pneumoniae
agaaaatggcaa

SEQ ID NO: 4 - DNA, 12 bp, Artificial Sequence
  rpsD - S. enterica
agaaaatggcaa

SEQ ID NO: 5 - DNA, 12 bp, Artificial Sequence
  rpsD - S. enterica, rtcA
agaaaatggcaa

SEQ ID NO: 6 - DNA, 12 bp, Artificial Sequence
  ffh - E. coli
gacaatgtttga

SEQ ID NO: 7 - DNA, 13 bp, Artificial Sequence
  ffh - K. pneumoniae
gacaatgtttgat

SEQ ID NO: 8 - DNA, 12 bp, Artificial Sequence
  ffh - S. enterica
gacaatgtttga

SEQ ID NO: 9 - DNA, 12 bp, Artificial Sequence
  lexA - E. coli
cggaatgaaagc

SEQ ID NO: 10 - DNA, 12 bp, Artificial Sequence
  lexA - K. pneumoniae
cggaatgaaagc

SEQ ID NO: 11 - DNA, 12 bp, Artificial Sequence
  lexA - S. enterica
cggaatgaaagc

SEQ ID NO: 12 - DNA, 12 bp, Artificial Sequence
  gyrB - E. coli
gttgatgtcgaa

SEQ ID NO: 13 - DNA, 12 bp, Artificial Sequence
  gyrB - K. pneumoniae
gttgatgtcgaa

SEQ ID NO: 14 - DNA, 13 bp, Artificial Sequence
  gyrB - S. enterica
cgttgatgtcgaa

SEQ ID NO: 15 - DNA, 12 bp, Artificial Sequence
  Peptide Nucleic Acid directed to alpha-folC
ataccatgatta

SEQ ID NO: 16 - DNA, 12 bp, Artificial Sequence
  Peptide Nucleic Acid directed to alpha-ffh
gacaatgtttga

SEQ ID NO: 17 - DNA, 12 bp, Artificial Sequence
  Peptide Nucleic Acid directed to alpha-lexA
cggaatgaaagc

SEQ ID NO: 18 - DNA, 12 bp, Artificial Sequence
  Peptide Nucleic Acid directed to alpha-gyrB
gttgatgtcgaa

SEQ ID NO: 19 - DNA, 12 bp, Artificial Sequence
  Peptide Nucleic Acid directed to alpha-rspD
agaaaatggcaa

SEQ ID NO: 20 - DNA, 60 bp, Artificial Sequence
  Antisense oligomer for alpha-rpsD
atttaggtgacactatagaagtggagaaagaaaatggcaagatatttgggtcctaagctc

SEQ ID NO: 21 - DNA, 60 bp, Artificial Sequence
  Antisense oligomer for alpha-lexA
atttaggtgacactatagaagcagggggcggaatgaaagcgttaacggccaggcaacaag

SEQ ID NO: 22 - DNA, 60 bp, Artificial Sequence
  Nonsense oligomer
gaattcgaattcggtcagtgcgtcctgctgatgtgctcagtatctctatcactgataggg

SEQ ID NO: 23 - protein, 10 aa, Artificial Sequence
  cell penetrating peptide
KFFKFFKFFK

SEQ ID NO: 24 - DNA, 20 bp, Artificial Sequence
  gyrB forward primer
cgggtccatagtggtttccc

SEQ ID NO: 25 - DNA, 20 bp, Artificial Sequence
  gyrB reverse primer
gtgagaaactgcgtggcttg

SEQ ID NO: 26 - DNA, 20 bp, Artificial Sequence
  folC forward primer
gctcaagcagttgttctgcc

SEQ ID NO: 27 - DNA, 20 bp, Artificial Sequence
  folC reverse primer
tctcaccgggcgtatgaaag

SEQ ID NO: 28 - DNA, 20 bp, Artificial Sequence
  ffh forward primer
ttccatacgcaccagcactt

SEQ ID NO: 29 - DNA, 20 bp, Artificial Sequence
  ffh reverse primer
cgcgcaggcagagaaattag

SEQ ID NO: 30 - DNA, 20 bp, Artificial Sequence
  rpsD forward primer
cagccaggttggcttttcac

SEQ ID NO: 31 - DNA, 20 bp, Artificial Sequence
  rpsD reverse primer
agaagcacgtcagctggtta

SEQ ID NO: 32 - DNA, 20 bp, Artificial Sequence
  lexA forward primer
gttaacggccaggcaacaag

SEQ ID NO: 33 - DNA, 20 bp, Artificial Sequence
  lexA reverse primer
tcaataacgcctttgcgtgc

SEQ ID NO: 34 - protein, 401 aa, Bacteriophage lambda
MKMPEKHDLLAAILAAKEQGIGAILAFAMAYLRGRYNGGAFTKTVIDATMCAIIAWFIRDLLDFAGLSSNLAYITSVFIGYIGTDSIGSLIKRFAAKKAGVEDGRNQSTGVPRYAGVVGGNRTSENQKSWLRHCRRRAIYLLRSPSQTCHAKPKTQINRRRTLPASFPLVGCLPQAAWPERLLSEKSGRCGIAADGAWRFTYDSWYPSGNRPLQQYLGFTAGRWLWSVRAGQPDCKIQRSGRNGQRDCMSRVTAIISALVICIIVCLSWAVNHYRDNAITYKAQRDKNARELKLANAAITDMQMRQRDVAALDAKYTKELADAKAENDALRDDVAAGRRRLHIKAVCQSVREATTASGVDNAASPRLADTAERDYFTLRERLITMQKQLEGTQKYINEQCR

SEQ ID NO: 35 - protein, 309 aa, Shigella flexneri
MVDLCNDLLSIKEGQKKEFTLHSGNKVSFIKAKIPHKRIQDLTFVNQKTNVRDQESLTEESLADIIKTIKLQQFFPVIGREIDGRIEILDGTRRRASAIYAGADLEVLYSKEYISTLDARKLANDIQTAKEHSIRELGIGLNFLKVSGMSYKDIAKKENLSRAKVTRAFQAASVPQEIISLFPIASELNFNDYKILFNYYKGLEKANESLSSTLPILKEEIKDLDTNLPPDIYKKEILNIIKKSKNRKQNPSLKVDSLFISKDKRTYIKRKENKTNRTLIFTLSKINKTVQREIDEAIRDIISRHLSSS

(19) United States
(12) Patent Application Publication
(10) Pub. No.: US 2021/0087565 A1
(43) Pub. Date: Mar. 25, 2021

(54) ANTISENSE ANTIBIOTICS AND BACTERIAL SECRETION BASED DELIVERY SYSTEM TO ELIMINATE DRUG-RESISTANT BACTERIA

(71) Applicant: The Regents of the University of Colorado, a body corporate, Dener, CO (US)

(72) Inventors: Anushree Chatterjee, Lafayette, CO (US); Jocelyn Campos, Boulder, CO (US); Kristen Alice Eller, Boulder, CO (US)

(21) Appl. No.: 17/048,948

(22) PCT Filed: Apr. 19, 2019
(86) PCT No.: PCT/US19/28392

§ 371 Date: Oct. 19, 2020

Related U.S. Application Data
(60) Provisional application No. 62/660,130, filed on Apr. 19, 2018.

(51) Int. Cl. C12N 15/113; C07K 14/00; C12N 15/70; C12N 15/74; C12N 15/85; A61K 47/64; A61K 47/54; A61K 35/741; A61K 35/744

(52) U.S. Cl. CPC C12N 15/113; C07K 14/003; C12N 15/70; C12N 15/74; C12N 15/746; C12N 15/85; A61K 2035/115; A61K 47/549; A61K 35/741; A61K 35/744; C12N 2310/11; C12N 2310/3181; C12N 2310/3513; A61K 47/64

(57) ABSTRACT

The present inventions relates to systems, methods and compositions for the rational design of a new classes of antibiotics targeting non-traditional pathways and genes including metabolism, cell signaling, and stress response using sequence-specific peptide nucleic acids (PNAs). The invention further includes systems, methods and compositions for the efficient delivery of PNAs to intracellular pathogens through a novel use of the bacterial secretion system in combination with a cell lysis switch.

This application claims the benefit of and priority to U.S. Provisional Patent Application No. 62/660,130, filed Apr. 19, 2018, which is incorporated herein by reference in its entirety.

### GOVERNMENT INTEREST

This invention was made with government support under grant number DARPA Young Faculty Award (D17AP00024), NSF Graduate fellowship (DGE 1144083), and a grant by the National Science Foundation (DGE1144083). The government has certain rights in the invention.

### SEQUENCE LISTING

The instant application contains a Sequence Listing which has been submitted electronically in ASCII format and is hereby incorporated by reference in its entirety.

### TECHNICAL FIELD

The inventive technology includes novel systems, methods and compositions for the treatment of bacterial infections, in particular multidrug-resistant infections. The invention further includes the novel use of antisense technology to rationally design antibiotics that can be configured to target antibiotic-resistant bacteria. Additional aspects of the invention include novel bacterial secretion delivery systems to deliver antisense oligomers to treat a multidrug-resistant infection.

### BACKGROUND

Multidrug-resistant (MDR) infections caused by antibiotic-resistant bacteria are threatening our ability to treat common infections causing an estimated 20 billion dollars in direct healthcare costs. This health crisis is due to the intersection of rapidly evolving antibiotic-resistant bacteria and the lack of new antibiotics being developed. In particular, bacterial pathogens in urgent need of effective antibiotics include Enterobacteriaceae such as carbapenem-resistant Enterobacteriaceae Escherichia coli and extended spectrum β-lactamase producing Klebsiella pneumoniae which were both recently designated priority one critical class by the World Health Organization. Current antibiotics are limited to small molecules targeting proteins within three main pathways in bacteria: cellular replication, protein biosynthesis, and cell wall biosynthesis. There are 303 essential genes in E. coli, with 139 of those existing in non-traditional antibiotic pathways such as metabolism, cell motility and secretion, and those of unknown function. Exploring non-traditional antibiotic targets expands the realm of potential therapeutics and introduces bacteria to new stresses to which they have not developed resistance.

Recent research efforts to identify novel small molecules by isolating microorganisms from specific niches (e.g., the discovery of teixobactin from soil microorganism) are promising but involve tedious, time-consuming screening processes. Additional strategies involving sequence specific targeting by antisense therapies have also been technically limited. For example, traditional sequence specific antisense therapies use reverse complement nucleic acids, which may be natural or synthetic, to bind the target RNA sequence via Watson-Crick base pairing and inhibit transcription and translation. Additional research in gene-specific/pathogen-specific targeting uses CRISPR-Cas9 technology offers potential, however, it relies on tedious cloning of guide RNA and pathogen specific CRISPR systems, all of which require time and cannot be readily translated to treat clinical bacterial strains.

One area of investigation has included the use of peptide nucleic acids (PNAs). Generally, PNAs are synthetic nucleic acids which have a modified protein-like backbone supporting the nucleic acid functional groups, providing increased stability in the cell due to no known enzymatic degradation. The neutral backbone of a PNA has reduced electrostatic repulsion resulting in increased nucleic acid binding affinity compared to natural nucleic acids, improving thermal stability of a duplex by 1-2° C. per base pair. Single-stranded PNA forms sequence-specific duplexes with RNA at higher stability compared to duplexes with DNA, leading to lower translation of the targeted RNA as opposed to transcriptional inhibition. PNAs have demonstrated antimicrobial activity against traditional antibiotic targets including β-lactamase drug resistance genes, 23S and 16S ribosomal RNA, and cell division proteins such as ftsZ. However, despite their promise, a systematic effort to target non-traditional antibiotic pathways has not been achieved. One major restraint on PNA therapies is their limited ability to be delivered into bacterial and mammalian cells due to the neutral nature of the molecule. This has diminished the overall effectiveness of PNA based therapy.

However, despite their promise, lack of a systematic effort to target non-traditional antibiotic pathways and poor transport properties of PNA has limited their application for antibiotic development. To overcome these limitations, the present invention presents an effective strategy involving a new class of rationally designed antibiotics targeting non-traditional pathways and genes including metabolism, cell signaling, and stress response using sequence-specific PNAs. As demonstrated below, these rationally designed PNAs showed successful growth inhibition against multi drug-resistant Enterobacteriaceae clinical isolates including carbapenem-resistant Escherichia coli, extended-spectrum beta-lactamase (ESBL) Klebsiella pneumoniae, New Delhi Metallo-beta-lactamase-1 carrying Klebsiella pneumoniae and MDR Salmonella enterica for 73% of treatments, with nearly 27% of the treatments leading to more than 97% growth inhibition. Despite presence of resistance genes, these rationally designed PNAs were capable of potentiating antibiotic activity in the clinical isolates. Finally, to address the poor transport of PNAs the present inventions demonstrates a novel use of the bacterial Type III secretion systems in combination with a cell lysis switch. As detailed below, this novel bacterial delivery system eliminated 99.6% of an intracellular Salmonella infection in human epithelial cells.

### SUMMARY OF THE INVENTION

The inventive technology generally relates to systems, methods and compositions for the treatment of bacterial infections, as well as novel use of antisense technology to rationally design antibiotics that can be applied to clinical cases and intracellular infections. One aim of the inventive technology is to generate new classes of antibiotics by targeting non-traditional pathways and genes using sequence-specific PNAs. Using predictive homology, such exemplary antisense therapeutics may be configured to target varied pathogenic species of bacteria, such as Enterobacteriaceae. Additional aims of the invention may include the use of antisense therapeutics configured to target specific pathogenic species of bacteria, such as carbapenem-resistant Escherichia coli, extended-spectrum beta-lactamase (ESBL) Klebsiella pneumoniae, New Delhi Metallo-beta-lactamase-1 carrying Klebsiella pneumoniae and MDR Salmonella enterica.

Another aim of the current invention may include the application of antisense therapeutics against a range of MDR bacteria. In one exemplary embodiment, PNAs designed to target essential genes in pathways including metabolism, cell signaling, and stress response may be capable of killing or inhibiting MDR bacterial strains.

Another aim of the invention may include the use of the application of antisense therapeutics to increase the effectiveness of antibiotics in treating bacterial infection, and in particular, MDR infection.

In yet another aim of the invention, a certain embodiment may use bacterial secretion systems to overcome limitations of transport typically associated with antisense therapeutics, such as PNAs, to treat an infection of bacterial pathogens. Certain embodiments of the invention may further include methods of delivering antisense therapeutics using a probiotic expressing a select bacterial secretion system, such as bacterial Type III or Type-IV secretion system.

One aim of the inventive technology includes systems, methods and compositions for the use of predictive homology to design broad-pathogen and gene-specific antisense RNA-inhibitors against of select pathogens and in particular clinical isolates of MDR bacteria.

Another aim of the inventive technology includes the rational design and application of antisense-PNA molecules in non-traditional antibiotic pathways to inhibit pathogen growth, and in particular cell growth of in MDR clinical isolates.

Another aim of the inventive technology includes the use of RNA-inhibitors, and preferably an antisense PNA directed to expression of a select pathogen gene, as a monotherapy wherein its inhibitory effect acts as an adjuvant or potentiator in combination with conventional antibiotics despite any preexisting resistance.

Another aim of the inventive technology includes implementation of rationally designed PNA design principles that can be used to create monotherapies by targeting tightly connected protein networks of particular biological pathways.

Additional aims of the inventive technology may include the use of RNA-inhibitors, and preferably an antisense PNA directed to expression of a select pathogen gene, as antibiotic potentiators which may be constructed by targeting multiple protein networks involved in diverse cellular processes to induce fitness impacts through multiplexed gene expression perturbations which may have negative effects on epistasis, and the slowing down bacterial evolution.

Another aim of the inventive technology includes systems, methods and compositions for a novel bacterial delivery and release system for overcoming the transport limitations of PNA to treat intracellular infections. In this preferred embodiment, a bacterial type III or IV secretion system may be used as a delivery method, and may further be combined with a holin-endolysin release switch system release system for effective therapeutic delivery. Such an embodiment may allow for a novel probiotic PNA delivery method for clinical applications.

Additional aims of the invention may be included in the following embodiments:

1. An antisense peptide nucleic acid configured to be complementary to at least one target sequence in a bacterial gene, wherein said peptide nucleic acid is configured to be capable of hybridizing to said target sequence and further inhibit expression of said bacterial gene.
2. The antisense peptide nucleic acid of embodiment 1 wherein said antisense peptide nucleic acid configured to be complementary to at least one target sequence in a bacterial gene has a sequence length selected from the group of: a 5-mers to a 40-mers; a 10-mers to a 15-mers; and a 12-mer.
3. The antisense peptide nucleic acid of embodiment 1 and further comprising a cell penetrating peptide conjugated to said antisense peptide nucleic acid sequence.
4. The antisense peptide nucleic acid of embodiment 3 wherein said cell penetrating peptide is selected from the group consisting of: (KFF)₃K; penetratin; NLS; TAT; Arg(9); D-Arg(9); 10HC; cyLoP-1; and Pep-1.
5. The antisense peptide nucleic acid of embodiment 4 wherein said cell penetrating peptide is conjugated to said antisense peptide nucleic acid sequence via a linker.
6. The antisense peptide nucleic acid of embodiment 5 wherein said linker is selected from the group consisting of: an AEEA-linker; an O-linker; an E-linker; a C6A linker; aC6SH linker; a X-linker; and C11SH linker.
7. The antisense peptide nucleic acid of embodiment 1 wherein said at least one target sequence in a bacterial gene comprises a start codon of a bacterial gene.
8. The antisense peptide nucleic acid of embodiment 1 wherein said bacterial gene comprises a bacterial gene related to one or more antibiotic pathways such that inhibition of said bacterial gene has a bactericidal and/or bacteriostatic effect.
9. The antisense peptide nucleic acid of embodiment 1 wherein said bacterial gene comprises a bacterial gene related to a non-traditional antibiotic pathway such that inhibition of said bacterial gene has a bactericidal and/or bacteriostatic effect.
10. The antisense peptide nucleic acid of embodiment 9 wherein said bacterial gene comprises a bacterial gene selected from the group consisting of: folC; ffh; lexA; gyrB; and rpsD.
11. The antisense peptide nucleic acid of embodiments 9 wherein said antisense peptide nucleic acid configured to be complementary to at least one target sequence in a bacterial gene comprises and antisense peptide nucleic acid configured to be complementary to a nucleic acid sequence selected from the group consisting of: SEQ ID NO. 1; SEQ ID NO. 2; SEQ ID NO. 3; SEQ ID NO. 4; SEQ ID NO. 5; SEQ ID NO. 6; SEQ ID NO. 7; SEQ ID NO. 8; SEQ ID NO. 9; SEQ ID NO. 10; SEQ ID NO. 11; SEQ ID NO. 12; SEQ ID NO. 13; and SEQ ID NO. 14.
12. The antisense peptide nucleic acid of embodiment 9 wherein said antisense peptide nucleic acid comprises an antisense peptide nucleic acid configured to be complementary to at least one target sequence in said folC gene and wherein said antisense peptide nucleic acid sequence is identified according to SEQ ID NO. 15.
13. The antisense peptide nucleic acid of embodiment 9 wherein said antisense peptide nucleic acid comprises an antisense peptide nucleic acid configured to be complementary to at least one target sequence in said ffh gene and wherein said antisense peptide nucleic acid sequence is identified according to SEQ ID NO. 16.
14. The antisense peptide nucleic acid of embodiment 9 wherein said antisense peptide nucleic acid comprises an antisense peptide nucleic acid configured to be complementary to at least one target sequence in said lexA gene and wherein said antisense peptide nucleic acid sequence is identified according to SEQ ID NO. 17.
15. The antisense peptide nucleic acid of embodiment 9 wherein said antisense peptide nucleic acid comprises an antisense peptide nucleic acid configured to be complementary to at least one target sequence in said gyrB gene and wherein said antisense peptide nucleic acid sequence is identified according to SEQ ID NO. 18.
16. The antisense peptide nucleic acid of embodiment 9 wherein said antisense peptide nucleic acid comprises an antisense peptide nucleic acid configured to be complementary to at least one target sequence in said rspD gene and wherein said antisense peptide nucleic acid sequence is identified according to SEQ ID NO. 19.
17. The antisense peptide nucleic acid of embodiment 1 wherein said bacterial gene comprises a bacterial gene selected from the group consisting of: a bacterial gene in E. coli, a bacterial gene in K. pneumoniae, and/or a bacterial gene in S. enteric.
18. The antisense peptide nucleic acid of embodiment 1 wherein said bacterial gene comprises a bacterial gene of a multi-drug resistant (MDR) bacteria.
19. The antisense peptide nucleic acid of embodiment 18 wherein said bacterial gene of a multi-drug resistant (MDR) bacteria is selected from the group consisting of: carbapenem resistant Enterobactericeae Klebsiella pneumonia (CREKP), MDR tuberculosis (MDRTB), MDR Salmonella enterica, MDR Salmonella typhimurium (MDRST), methicillin-resistant Staphylococcus aureus (MRSA), vancomycin-resistant S. aureus (VRSA), extended spectrum κ-lactamase Klebsiella pneumoniae (ESBL K. pneumoniae), vancomycin-resistant Enterococcus (VRE), carbapenem-resistant Enterobacteriaceae Escherichia coli (CRE E. coli), MDR Escherichia coli (MDR E. coli), New-Delhi metallo-β-lactamase producing Klebsiella pneumoniae (NDM-1 K. pneumoniae), and MDR Acinetobacter baumannii (MRAB).
20. A method of treating a bacterial infection comprising administering to a patient a therapeutically effective amount of the antisense peptide nucleic acid of embodiment 1.
21. The method of embodiment 20 and further comprising the step of administering to a patient a therapeutically effective amount antisense peptide nucleic acid and further comprising the step of administering to a patient a therapeutically effective amount at least one antibiotic compound.
22. The method of embodiment 22 wherein said antibiotic compound is selected from the group of: penicillins; cephalosporins; carbacephems; cephamycins; carbapenems; monobactams; aminoglycosides; glycopeptides; quinolones; tetracyclines; macrolides; and fluoroquinolones.
23. An antisense peptide nucleic acid selected from the group consisting of: the sequence according to SEQ ID NO. 15, the sequence according to SEQ ID NO. 16, the sequence according to SEQ ID NO. 17, the sequence according to SEQ ID NO. 18, and the sequence according to SEQ ID NO. 19.
24. The composition of embodiment 23 wherein said antisense peptide nucleic acid is introduced into a bacterium having an endogenous or exogenous expresses Type III secretion system configured to inject said antisense peptide nucleic acid into a eukaryotic cell.
25. The bacterium of embodiment 24 wherein said bacterium comprises a genetically engineered bacterium having a polynucleotide coding sequence operably linked to an inducible promoter encoding a heterologous cell lysis kill switch, having at least one heterologous cell lysis protein.
26. The bacterium of embodiment 25 wherein said heterologous cell lysis protein comprises a holin-endolysin protein according to SEQ ID NO. 34.
27. A method of delivering an antisense peptide nucleic acid to a host comprising the steps:
generating at least one antisense peptide nucleic acid configured to be complementary to at least one target sequence in a bacterial gene, wherein said antisense peptide nucleic acid is configured to be capable of hybridizing to said target sequence and inhibit expression of said bacterial gene;
transforming a delivery bacteria with a polynucleotide coding sequence operably linked to an inducible promoter encoding a heterologous cell lysis kill switch, wherein said cell lysis kill switch comprises a polynucleotide expressing:
   a heterologous holin-endolysin construct;
   a heterologous bacterial secretion system;
   a heterologous transcriptional activator that induces expression of said heterologous bacterial secretion system;
introducing said antisense peptide nucleic acid into said transformed delivery bacteria having said heterologous cell lysis kill switch; and
introducing a therapeutically effective amount of said transformed delivery bacteria having one or more of said antisense peptide nucleic acids and further allowing the transport of said antisense peptide nucleic acid into a host cell through said bacterial secretion system and through cell membrane pores formed by said heterologous cell lysis kill switch;
lysing said transformed delivery bacteria through the action of said heterologous cell lysis kill switch.
   28. The method of embodiment 27 wherein said bacterial secretion system comprises a bacterial secretion system selected from the group consisting of: a Type-III secretion system, and Type-IV secretion system.
   29. The method of embodiment 27 wherein said transformed delivery bacteria is a probiotic to the host, symbiotic to the host, or endosymbiotic with the host.
   30. The method of embodiment 27 wherein said step of introducing a therapeutically effective amount of said transformed delivery bacteria comprises the step of administering a therapeutically effective amount of said antisense peptide nucleic acid to a host cell to treat a bacterial infection.
   31. The method of embodiment 30 wherein said bacterial infection comprises a multi-drug resistant (MDR) bacterial infection.
   32. The method of embodiment 27 wherein said heterologous transcriptional activator comprises heterologous VirB transcriptional activator according to amino acid sequence according to SEQ ID NO. 35.
   33. The method of embodiment 32 wherein said heterologous holin-endolysin construct comprises a holin-endolysin construct according to amino acid sequence SEQ ID NO. 34.
   34. The method of embodiment 33 wherein said inducible promoter comprises a promoter that is induced by entry into a host cell.
   35. The method of embodiment 34 wherein said promoter that is induced by entry into a host cell comprises an Ipac promoter from Shigella flexneri induced by entry into a mammalian host cell.
   36. The method of embodiment 27 and further comprising a cell penetrating peptide conjugated to said antisense peptide nucleic acid configured to be complementary to at least one target sequence in a bacterial gene and wherein said cell penetrating peptide is selected from the group consisting of: (KFF)₃K; penetratin; NLS; TAT; Arg(9); D-Arg(9); 10HC; cyLoP-1; and Pep-1.
   37. The method of embodiment 36 wherein said cell penetrating peptide is conjugated to the antisense peptide nucleic acid via a linker and wherein said linker is selected from the group consisting of: an AEEA-linker, an O-linker; an E-linker; a C6A linker; aC6SH linker; a X-linker; and C11SH linker.
   38. The method of embodiment 27 wherein said target sequence in a bacterial gene comprises a start codon of a bacterial gene.
   39. The method of embodiment 27 wherein said bacterial gene comprises a bacterial gene related to one or more antibiotic pathways such that inhibition of said bacterial gene has a bactericidal and/or bacteriostatic effect.
   40. The method of embodiment 39 wherein said bacterial gene comprises a bacterial gene related to a non-traditional antibiotic pathway such that inhibition of said bacterial gene has a bactericidal and/or bacteriostatic effect.
   41. The method of embodiment 40 wherein said bacterial gene comprises a bacterial gene selected from the group consisting of: folC; ffh; lexA; gyrB; and rpsD.
   42. The method of embodiment 40 wherein said antisense peptide nucleic acid configured to be complementary to at least one target sequence in a bacterial gene comprises and antisense peptide nucleic acid configured to be complementary to a nucleic acid sequence selected from the group consisting of: SEQ ID NO. 1; SEQ ID NO. 2; SEQ ID NO. 3; SEQ ID NO. 4; SEQ ID NO. 5; SEQ ID NO. 6; SEQ ID NO. 7; SEQ ID NO. 8; SEQ ID NO. 9; SEQ ID NO. 10; SEQ ID NO. 11; SEQ ID NO. 12; SEQ ID NO. 13; and SEQ ID NO. 14.
   43. The method of embodiment 40 wherein said bacterial gene is folC wherein said antisense peptide nucleic acid comprises an antisense peptide nucleic acid configured to be complementary to at least one target sequence in said folC gene and wherein said antisense peptide nucleic acid sequence is identified according to SEQ ID NO. 15.
   44. The method of embodiment 40 wherein said antisense peptide nucleic acid comprises an antisense peptide nucleic acid configured to be complementary to at least one target sequence in said ffh gene and wherein said antisense peptide nucleic acid sequence is identified according to SEQ ID NO. 16.
   45. The method of embodiment 40 wherein said antisense peptide nucleic acid comprises an antisense peptide nucleic acid configured to be complementary to at least one target sequence in said lexA gene and wherein said antisense peptide nucleic acid sequence is identified according to SEQ ID NO. 17.
   46. The method of embodiment 40 wherein said antisense peptide nucleic acid comprises an antisense peptide nucleic acid configured to be complementary to at least one target sequence in said gyrB gene and wherein said antisense peptide nucleic acid sequence is identified according to SEQ ID NO. 18.
   47. The method of embodiment 40 wherein said antisense peptide nucleic acid comprises an antisense peptide nucleic acid configured to be complementary to at least one target sequence in said rspD gene and wherein said antisense peptide nucleic acid sequence is identified according to SEQ ID NO. 19.
   48. The method of embodiment 27 wherein said bacterial gene comprises a bacterial gene selected from the group consisting of: E. coli, K. pneumoniae, and S. enterica.
   49. The method of embodiment 27 wherein said bacterial gene comprises a bacterial gene of a multi-drug resistant (MDR) bacteria.
   50. The method of embodiment 49 wherein said bacterial gene of a multi-drug resistant (MDR) bacteria is selected from the group consisting of: carbapenem resistant Enterobactericeae Klebsiella pneumonia (CREKP), MDR tuberculosis (MDRTB), MDR Salmonella enterica, MDR Salmonella typhimurium (MDRST), methicillin-resistant Staphylococcus aureus (MRSA), vancomycin-resistant S. aureus (VRSA), extended spectrum β-lactamase Klebsiella pneumoniae (ESBL K. pneumoniae), vancomycin-resistant Enterococcus (VRE), carbapenem-resistant Enterobacteriaceae Escherichia coli (CRE E. coli), MDR Escherichia coli (MDR E. coli), New-Delhi metallo-β-lactamase producing Klebsiella pneumoniae (NDM-1 K. pneumoniae) and MDR Acinetobacter baumannii (MRAB).
   51. The method of embodiment 27 and further comprising the step of co-administering a therapeutically effective amount of at least one antibiotic compound.
   52. The method of embodiment 51 wherein said antibiotic compound is selected from the group of: penicillins; cephalosporins; carbacephems; cephamycins; carbapenems; monobactams; aminoglycosides; glycopeptides; quinolones; tetracyclines; macrolides; and fluoroquinolones.
   53. A method for developing an antibacterial antisense therapeutic comprising:
identifying at least one target gene involved in bacterial fitness; and
designing an antisense peptide nucleic acid complimentary to an mRNA sequence of the at least one target gene thereby developing an antibacterial antisense therapeutic.
   54. A method for delivering an antibacterial antisense therapeutic comprising:
transforming a delivery bacteria with a polynucleotide coding sequence operably linked to promoter encoding a heterologous antisense molecule configured to be complementary to at least one target sequence in a pathogen gene;
transforming a delivery bacteria with a polynucleotide coding sequence operably linked to promoter encoding a heterologous cell lysis kill switch.
   55. A method for developing an antibacterial antisense therapeutic comprising:
establishing a bacteria having an endogenous bacterial secretion system;
transforming a delivery bacteria with a polynucleotide coding sequence operably linked to promoter encoding a heterologous antisense molecule configured to be complementary to at least one target sequence in a pathogen gene;
transforming a delivery bacteria with a polynucleotide coding sequence operably linked to promoter encoding a heterologous cell lysis peptide.
   56. A method of delivering a therapeutic molecule to comprising the steps:
generating a therapeutic molecule configured to inhibit expression of a pathogen bacterial gene;
transforming a delivery bacteria with a polynucleotide coding sequence operably linked to a promoter encoding a heterologous cell lysis kill switch;
introducing said therapeutic molecule into said transformed delivery bacteria; and
introducing a therapeutically effective amount of said transformed delivery bacteria having one or more of said antisense peptide nucleic acids and further allowing the transport of said therapeutic molecule into a host cell through the action of said heterologous cell lysis kill switch; and
lysing said transformed delivery bacteria through the action of said heterologous cell lysis kill switch.
   57. The method of embodiment 56 wherein said therapeutic molecule comprises a therapeutic molecule selected from the group consisting of: an antisense therapeutic molecule, a peptide nucleic-acid molecule; a DNA polynucleotide molecule; and RNA polynucleotide molecule; a single-stranded DNA polynucleotide molecule; a single stranded RNA polynucleotide molecule; an interfering RNA molecule; a vaccine; a chemotherapy molecule; a protein inhibitor; a gene editing molecule; a CRISPR/Cas9 system gene or protein; a gene; an antibiotic; a polypeptide; an antibody, therapeutic peptides, Zinc Fingers endonuclease system gene or protein, TALENS endonuclease system gene or protein, and/or an plasmid.
   58. The method of embodiment 56 wherein said heterologous cell lysis kill switch comprises:
a heterologous cell lysis peptide;
a heterologous bacterial secretion system; and
a heterologous transcriptional activator that induces expression of said heterologous bacterial secretion system.
   59. The method of embodiment 58 wherein said heterologous bacterial secretion system comprises a heterologous Type III or Type-IV secretion system.
   60. The method of embodiment 58 wherein said heterologous cell lysis peptide comprises the amino acid sequence according to SEQ ID NO. 34.
   61. The method of embodiment 58 heterologous transcriptional activator that induces expression of said heterologous bacterial secretion system comprises the amino acid sequence according to SEQ ID NO. 35.
   62. The method of embodiment 56 wherein said promoter comprises an inducible promoter that is induced by entry into a host cell.
   63. The method of embodiment 62 wherein said promoter that is induced by entry into a host cell comprises an Ipac promoter from Shigella flexneri induced by entry into a mammalian host cell.
   64. The method of embodiment 56 and further comprising a cell penetrating peptide conjugated to said therapeutic molecule and wherein said cell penetrating peptide is selected from the group consisting of: (KFF)₃K; penetratin; NLS; TAT; Arg(9); D-Arg(9); 10HC; cyLoP-1; and Pep-1.
   65. The method of embodiment 64 wherein said cell penetrating peptide is conjugated to said therapeutic molecule via a linker and wherein said linker is selected from the group consisting of: an AEEA-linker, an O-linker; an E-linker; a C6A linker; aC6SH linker; a X-linker; and C11SH linker.
   66. The method of embodiment 56 wherein said transformed delivery bacteria is a probiotic to the host, symbiotic to the host, or endosymbiotic with the host.
   67. The method of embodiment 56 and further comprising the step of co-administering a therapeutically effective amount of at least one antibiotic compound to said host.
   68. The method of embodiment 56 wherein said therapeutic molecule comprises an antisense peptide nucleic acid.
   67. The method of embodiment 68 wherein said antisense peptide nucleic acid comprises an antisense peptide nucleic acid selected from the group consisting of: an antisense peptide nucleic acid according to SEQ ID NO. 15, an antisense peptide nucleic acid according to SEQ ID NO. 16, an antisense peptide nucleic acid according to SEQ ID NO. 17, an antisense peptide nucleic acid according to SEQ ID NO. 18, and an antisense peptide nucleic acid according to SEQ ID NO. 19.

Additional aims of the inventive technology will be evident from the detailed description and figures presented below.

### BRIEF DESCRIPTION OF DRAWINGS

The above and other aspects, features, and advantages of the present disclosure will be better understood from the following detailed descriptions taken in conjunction with the accompanying figures, all of which are given by way of illustration only, and are not limiting the presently disclosed embodiments, in which:

FIG. 1. Rational design of PNAs across multiple pathogens against non-traditional antibiotic pathways and novel genes to inhibit multi-drug resistant bacteria. (A) Bioinformatic toolbox predicts PNAs that can target essential genes in one or more Enterobacteriaceae including E. coli, K. pneumoniae (KPN), and S. enterica (STm). E. coli essential genes are identified and screened for internal off-targets. Candidates without off-targets are narrowed to those with homology among KPN and STm. Of the 71 final candidates that meet the thermodynamic requirements for experimental conditions five gene targets were randomly chosen for assessment. (B) Most of the PNAs have homology to all three Enterobacteriaceae in this study, except α-folC which is designed to be specific to E. coli. The 12-mer sequence of each PNA is shown N terminus to C terminus. Not shown is the (KFF)₃K cell penetrating peptide (CPP) attached at the N-terminus via an O-linker. (C) PNAs are designed to be added exogenously to cultures of the target bacteria. (D) All PNAs are conjugated to the CPP via an AEEA-linker for increased transport into bacteria. (E) Upon bacterial uptake, the PNAs inhibit translation by binding to their corresponding mRNAs (e.g., by binding to AUG start codon). (F) Each PNA targets a different bacterial essential function. PNAs α-folC, α-ffh, and α-lexA target novel pathways of metabolism, signal recognition, and stress response respectively. PNAs α-gyrB and α-rpsD target novel genes in traditional antibiotic pathways.

FIG. 2. Antisense antimicrobials diminish the growth of a range of clinical isolates. (A) Antibiotic resistance characterization of clinical isolates of CRE E. coli, MDR E. coli, ESBL KPN, NDM-1 KPN, and MDR S. typhimurium. (Left) Antibiotic resistance characterization of clinical strains used in this study; “R” indicates drug-resistance, “S” indicates sensitivity, and “I” indicates intermediate resistance. Sensitive, Intermediate, and Resistant breakpoints are provided in Table 4. Nine antibiotics of varied mechanisms and classes were tested including penicillins (ampicillin, AMP), cephalosporins (ceftriaxone, FRX), carbapenems (meropenem, MER), aminoglycosides (gentamicin, GEN and kanamycin, KAN), tetracyclines (tetracycline, TET), fluoroquinolones (ciprofloxacin, CIP), quinolones (nalidixic acid, NXA), and phenicols (chloramphenicol, CHL). (Right) Unique antibiotic resistance genes identified in clinical isolates. (B) Predicted homology (shading) and significant growth inhibition (*) of the clinical isolate. (C) Normalized growth (ratio of optical density of treatment to no treatment at 16 hours) of clinical isolates in the presence of treatment with 10 μM of the indicated antisense-PNA. All data shown are the average of 3 biological replicates with standard deviation shown as error bars. Significance (represented by an asterisk, p-val<0.05) was determined relative to control nonsense PNA with a scrambled sequence (indicated as α-nonsense) with a 95% confidence interval. (D) STRING database data shown in FIG. 21 was used to determine the average protein interactions of the protein partners directly impacted by PNA knockdown (y-axis) compared to the corresponding normalized growth (x-axis). A linear fit was performed and correlation and significance (p=0.008) presented. As the α-folC was not able to bind to sequences in Klebsiella or Salmonella, this data was excluded for these organisms.

FIG. 3. Antisense-PNA acts as a potentiator and adjuvant with small-molecule traditional antibiotics. (A-B) Addition of α-gyrB potentiates activity of antibiotics Chloramphenicol (CHL) (A) and Gentamicin (GEN) (B) in CRE E. coli. (C-E) Potentiation of tetracycline (TET) activity in ESBL KPN with addition of either α-ffh (C), α-lexA (D), or α-gyrB (E). (F) Addition of α-rpsD potentiates activity of Meropenem (MER) in NDM-1 KPN. All graphs in (A-F) show normalized growth (optical density for respective strain at 24 h with respect to time t=0) for no treatment (left), 10 μM respective PNA (middle left), antibiotic (middle right), and combination of PNA and antibiotic (right). Antibiotic concentrations were (from top to bottom) 2 μg/mL tetracycline (TET), 4 μg/mL gentamicin (GEN), 8 μg/mL chloramphenicol (CHL), and 8 μg/mL meropenem (MER). Data shown are the average of 3 biological replicates with standard deviation shown as error bars. Significance (represented by an asterisk, p-val<0.05) was determined relative to monotherapy treatments of PNA or antibiotic with a 95% confidence interval. S-values obtained using the Bliss Independence model shown above each graph indicate a synergistic interaction between PNA and antibiotic.

FIG. 4. Establishing the bacterial Type III secretion system (T3SS) for PNA transport. (A) Schematic showing enhancement of PNA delivery into HeLa cells using the bacterial T3SS of Salmonella enterica serovar typhimurium (STm) for treatment of an intracellular infection of STm. HeLa cells were seeded and allowed to grow for 24 h. SL1344 GFP STm was added at a MOI of 10:1 and allowed to infect HeLa cells for 45 min following two separate conditions: (Top) No PNA is added during this time, termed here as Naked PNA treatment, (Bottom) PNA is added at the time of infection at t=0 to utilize the T3SS, termed here as T3SS-PNA treatment. The T3SS-PNAs enter Salmonella during 45 minutes of incubation due to transport enhanced by the cell penetrating peptides, followed by entry into host cells via T3SS encoding and PNA carrying STm. (Top and Bottom). After 45 minutes infected HeLa cells are washed and treated with media containing gentamicin for 75 minutes to kill extracellular bacteria. The cells are then incubated for 18 h with media containing gentamicin following the two separate conditions: (Top) PNA supplied after infection for Naked PNA treatment. (Bottom) No PNA is added after infection. Post 18 hours of treatment HeLa cells are fixed for imaging or lysed for Colony forming Unit (CFU) analysis. (B) Representative images of uninfected (Top left), and infected HeLa cells without treatment (Bottom left). Top (right) panels show representative images of infected HeLa cells following Naked-PNA treatment. Bottom (right) panels show representative images of infected HeLa cells following T3SS-PNA treatment. HeLa cells are stained with nucleic stain DAPI (blue), and membrane stain Phalloidin (pink), and green pixels represent intracellular SL1344 S. typhimurium fluorescing GFP (green). Images show an evident decrease in GFP expressing Salmonella during T3SS-α-rpsD or T3SS-α-lexA treatment compared to no treatment or with Naked-PNA treatment. (C) Intracellular S. typhimurium load (CFU/mL) in presence or absence of treatment. Significant reduction in STm CFU in HeLa cells when PNA is delivered using T3SS. Naked-PNA treatment does not produce the significant therapeutic effect.

FIG. 5. PNA binding to off-target start codons. (A) The set of potential PNA candidates identified from the Keio collection were screened against the entire E. coli MG1655 genome to identify the distribution of off-target alignments when allowing for zero base mismatches in the alignment (left) and one base mismatch (right). In the 0-basepair mismatch analysis, all chosen PNAs that were tested had no other start codon alignments within the genome, whereas in the one base mismatch alignment, the number of start codon alignments distribute across zero to five alignments. (B) Electrophoretic mobility shift assay (EMSA) depicting 60 nucleotide single-stranded DNA fragments containing the complementation site for their corresponding PNA. Nonsense DNA was a random 60 nucleotide single-stranded DNA sequence that contained no complementation sites for PNA binding. Lanes with only PNA showed no bands because the SYBR-Gold® stain intercalates between the base pairs and the PNA has no secondary structure (bands not shown). Bands corresponding to PNA+complementary DNA are shifted above the unbound DNA (red triangles). Corresponding PNA+nonsense DNA bands are seen in lanes 3 and 5, with lane 3 having slight cross-over bands coming from lane 2. All lanes are from the same gel.

FIG. 6. Homology of antisense-PNA RNA-inhibitors in clinical isolates. After sequencing, UGENE was used to search for the 12 nucleotide antisense-PNA targets in the gene of interest. The targets, predicted using the non-pathogenic and drug-sensitive strains, were present in all cases. Sequences are listed 5′-3′ with the antisense-PNA target bold and underlined with the translation start codon italicized. Grey indicates matching sequences.

FIG. 7. Dose response of various PNA. Optical density of a 1:10,000 dilution of MG6155 from overnight liquid culture, treated with various concentrations of antisense inhibitors was measured for 3 biological replicates with error bars as standard deviations. Normalized growth was measured as the optical density at 22 hours normalized to no treatment. An asterisk (*) indicates a significant difference (p<0.05) as compared to no treatment for each inhibitor.

FIG. 8. Antisense Inhibitors targeting novel pathways can inhibit growth of E. coli. z. (A) Growth curves of E. coli MG1655 from a 1:10,000 dilution from an overnight culture, normalized to time t=0 with 10 μM of respective antisense inhibitor. Inhibitors α-lexA and α-rpsD show complete suppression of cell growth. (B) Colony forming units per milliliter of MG1655 E. coli for respective treatment as a function of time. The CFU/mL at t=0 represents the starting culture after a 1:100,000 dilution from overnight culture. MG1655 treatment with 10 μM α-rpsD resulted in 0 CFU/mL within 2 hours of treatment. Pound sign (#) represents significantly different from t=0 and asterisk (*) indicates significantly different from no treatment at each specified time point. Significance is measured as p<0.05.

FIG. 9. Growth curves of MDR clinical isolates with respective PNA monotherapy treatment. Growth curves shown are the average of three biological replicates normalized to the optical density at t=0 with error bars as standard deviation. Each antisense inhibitor treatment (10 μM) shown with no treatment growth curves in grey for comparison. Data were used in FIG. 2B-D and FIG. 3 of the main text. An asterisk (*) indicates a significant difference (p<0.05) at 16 hours compared to no treatment at 16 hours.

FIG. 10. Growth curves of MDR clinical isolates subjected to antibiotic and PNA combination treatment. Optical density growth curves of each multidrug resistant bacteria from a 1:10,000 dilution of overnight liquid cultures, normalized to t=0. Growth curves shown are of three biological replicates with error bars representing standard deviation; the data shown was used in FIG. 3 of the main text, and an asterisk (*) indicates significant inhibition (p<0.05) at 16 hours as compared to no treatment. All conditions were treated with 10 μM of the indicated PNA. Antibiotic concentrations were (from top to bottom) 2 μg/mL tetracycline (TET), 4 μg/mL gentamicin (GEN), 8 μg/mL chloramphenicol (CHL), and 8 μg/mL meropenem (MER).

FIG. 11. T3SS-PNA treatment eliminates Salmonella infection of osteoblast cells. Osteoblast precursor cells were grown for 24 hours on tissue culture treated 96-well plates prior to infection with SL1344 at a multiplicity of infection of 30. (A) The antisense inhibitor α-rpsD was added at 10 μM either without infection (No Treatment), after infection (Naked), or during infection (T3SS Transport). (B) After treatment for 18 hours the infection was imaged in brightfield and GFP channel. GFP expression corresponds to an intracellular infection with red arrows indicating hard to see GFP expression.

FIG. 12. Cytotoxicity measurement of varying concentration of PNA in HeLa cells. HeLa cells were plated on tissue culture treated 96-well plates at 4,500 cells per well. Varying concentrations of α-lexA and α-nonsense were added exogenously after 24 hours of growth and incubated in growth conditions for 18 hours. Cytotoxicity was measured using a lactate dehydrogenase assay and absorbance read at 450 nm with a blank (growth media with lactate dehydrogenase assay) subtracted. All conditions were compared to the negative control indicated by a minus sign (−) and no significant difference, indicated by p>0.05, was seen for α-lexA up to 40 μM and α-nonsense up to 80 μM. Error bars are standard deviation.

FIG. 13. Effect of 45-minute incubation with PNA treatment during infection. (A) Individual colonies of SL1344 were picked off of solid media and grown for 16 hours in luria broth supplemented with 30 μg/mL streptomycin, diluted 1:10, and regrown for 4 hours. After regrowth bacteria was diluted to a concentration equivalent to a 30 multiplicity of infection of 200,000 cells/mL in 100 μL (conditions of HeLa cell infection after 24 hours growth). Cultures were suspended in phosphate buffered saline and treated with 10 μM of either α-lexA, α-rpsD, or plain phosphate buffered saline for 45 minutes before serial dilution, plated on solid media (40 μg/mL streptomycin), and incubated for 16 hours. Colony forming units were counted to enumerate percent infection with respect to no treatment. There is no significant difference (p>0.05) between α-lexA and α-rpsD at 45 minutes of treatment. (B) Cultures were grown and treated as described for panel A except that prior to treatment an aliquot was removed from each biological replicate as a no treatment condition for that biological replicate. Percent infection was then calculated in comparison to each replicates' no treatment. Error bars are standard deviation for three biological replicates.

FIG. 14. Combining the bacterial Type III secretion system (T3SS) and the holing/endolysin kill switch for PNA delivery and release. (A) Schematic showing the PNA deliver and release approach using the T3SS and Holin systems respectively to treat an intracellular infection of HeLa cells. HeLa cells were seeded 24 hours prior to infection. A 4:1 mixture of SL1344-Holin to SL1344-mCherry cells with (Bottom) and without (Top) 45 minute PNA pretreatment used to infect HeLa cells for 45 minutes. (Bottom) The PNA enters SL1344-Holin during the 45 minute pretreatment and is carried into the HeLa cell during infection. Following infection and gentamicin protection, the PNA is released from the SL1344-Holin during the 18-hours of treatment to target the SL1344-mCherry population. After 18-hours of incubation the HeLa cells are lysed to determine the number of intracellular colony forming units. (B) Normalized intracellular bacterial load represented as the ratio of normalized SL1344-mCherry:

(CFU_{SL1344-mCherry}^{Lysed}/CFU_{SL1344-mCherry}^{Infected With})

to normalized SL1344-Holin:

(CFU_{Holin}^{Lysed}/CFU_{Holin}^{Infected With})

where triangles represent individual biological replicates. (C) Normalized intracellular load of each HeLa biological replicate presented as the CFU count of each bacterial strain divided by the total intracellular bacterial load lysed from the individual HeLa cells. Above each bar is the associated MOI that was used to infect the specific biological replicate. Data shown are the average of 3 biological replicates with standard deviation shown as error bars. Significance (represented by an asterisk, p-val<0.05) was determined relative to no treatment.

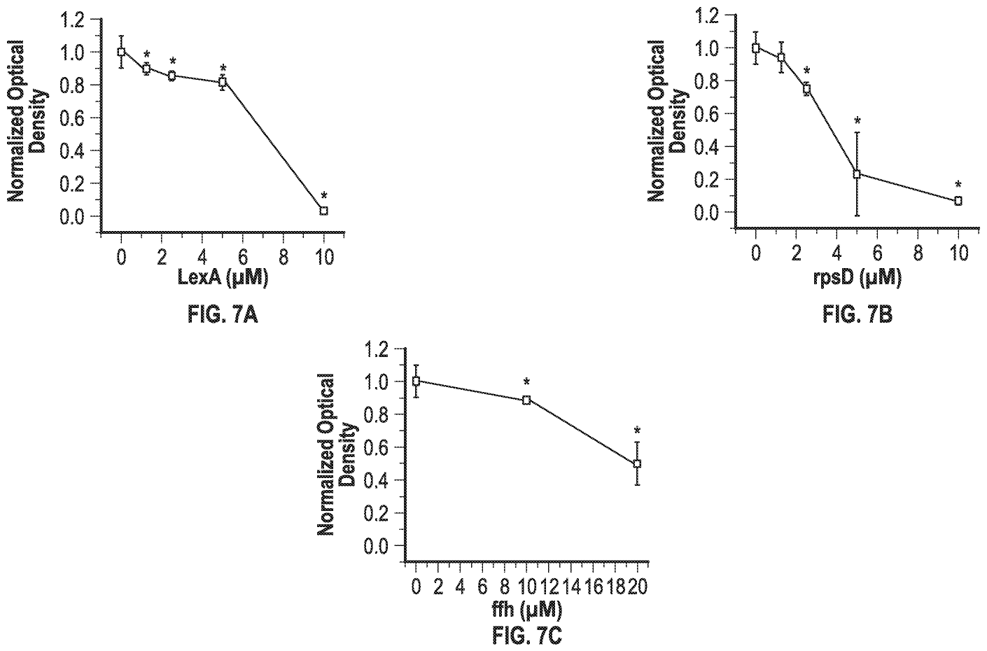

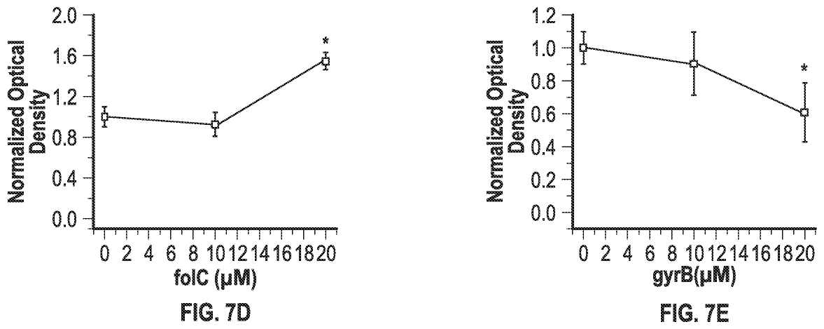

FIG. 15. qPCR RNA Expression and PNA efficacy correlates with RNA concentration. (A) Mean expression levels of three of the five PNA targets as measured by qPCR with respect to the expression of the housekeeping gene (cysG). (B) The average number of mRNA molecules per cell in exponential phase MG1655 was extracted from transcriptome of E. coli. We compared this data to the estimated concentration of PNA that caused MG1655 growth to fall to normalized OD 0.8, based on linear fits of graphs from Supplementary FIG. 7. These data sets were plotted on the y- and x-axis respectively, and an exponential decay fit was performed on the data. The resulting correlation is plotted here, along with the corresponding equation and its R² value.

FIG. 16. Predicted RNA folding of ffh mRNA. The entire theoretical mRNA sequence from E. coli MG1655 was analyzed using the RNAfold WebServer. Folding is based on minimum free energy structures. Red shades indicate lower entropy, or areas of low conformational flexibility. The specific binding site of the PNA sequence is shown to the right.

FIG. 17. Predicted RNA folding of folC mRNA. The entire theoretical mRNA sequence from E. coli MG1655 was analyzed using the RNAfold WebServer. Folding is based on minimum free energy structures. Red shades indicate lower entropy, or areas of low conformational flexibility. The specific binding site of the PNA sequence is shown to the right.

FIG. 18. Predicted RNA folding of gyrB mRNA. The entire theoretical mRNA sequence from E. coli MG1655 was analyzed using the RNAfold WebServer. Folding is based on minimum free energy structures. Red shades indicate lower entropy, or areas of low conformational flexibility. The specific binding site of the PNA sequence is shown to the right.

FIG. 19. Predicted RNA folding of lexA mRNA. The entire theoretical mRNA sequence from E. coli MG1655 was analyzed using the RNAfold WebServer³. Folding is based on minimum free energy structures. Red shades indicate lower entropy, or areas of low conformational flexibility. The specific binding site of the PNA sequence is shown to the right.

FIG. 20. Predicted RNA folding of rpsD mRNA. The entire theoretical mRNA sequence from E. coli MG1655 was analyzed using the RNAfold WebServer³. Folding is based on minimum free energy structures. Red shades indicate lower entropy, or areas of low conformational flexibility. The specific binding site of the PNA sequence is shown to the right.

FIG. 21. Predicted protein interaction networks of each PNA gene target. Known protein-protein interactions (obtained from the STRING database) between the gene targeted by each PNA and other proteins throughout the cell are shown as string maps. String maps present connections based on confidence, with darker lines indicating greater confidence in the existence of an interaction and node color indicating “closeness” to the target protein. The total number of nodes is tabulated to the left for each network, as along with the cluster coefficient of the overall network representing the “tightness” of the proteins in function. Networks were constructed for standard genomes of E. coli, K. pneumoniae, and S. typhimurium. FolC networks were excluded for the latter two genomes, as the designed α-folC PNA had no homology to the corresponding genes in these organisms. Networks were constructed using information from the String database based on co-expression, co-occurrence, gene fusion, experimental validation, databases, text mining, and neighborhood. A medium confidence interaction score of 0.400 threshold was established.

FIG. 22. Pathways impacted by PNA gene expression knockdown in E. coli, K. Pneumoniae, and S. typhimurium. The STRING maps created in FIG. 21 were used to extract the known cellular pathway that each related protein is involved in, based on the Kyoto Encyclopedia of Genes and Genomes (KEGG). Overarching cellular pathways are grouped, color coded, and each affected sub pathway is categorized to its larger pathway via color coding. Pathways that are significantly overrepresented (p<0.05) in the overall network were identified, and the number of proteins in each pathway were determined. A color scale was created showing the relative number of proteins in each of these pathways. A logarithmic transformation was used to ensure that pathways with low protein counts were still visible.

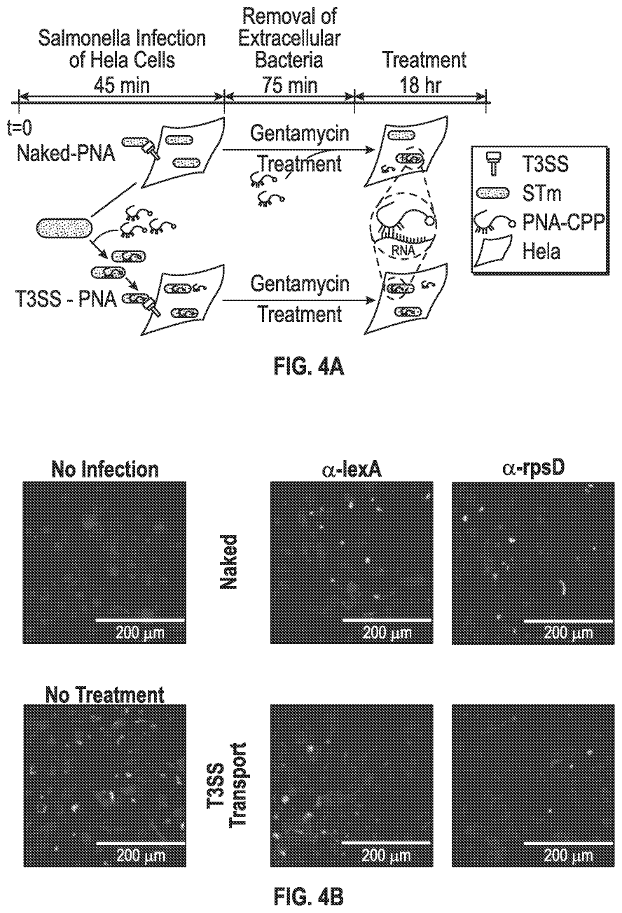

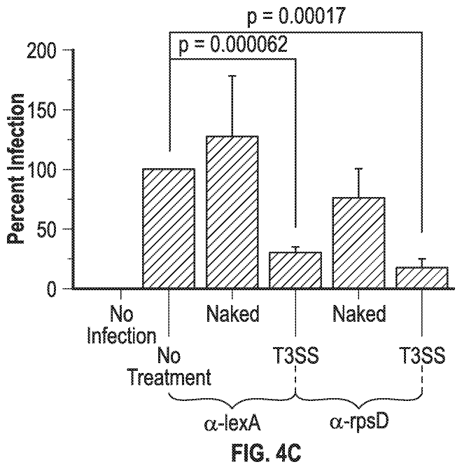

FIG. 23. Raw CFU/mL of HeLa infection treated by T3SS. Intracellular SL1344 colonies were enumerated by lysing with 30 μL of 0.1% Triton for 15 minutes at room temperature then diluted 1:10 by 270 μL addition of DPBS. The lysate was serially diluted 1:10 in 100 μLs and 10 μL plated onto LB agar with 40 μg/mL of Streptomycin. Following overnight growth colonies were counted to determine the CFU/mL. Shown are individual HeLa biological replicates shown in FIG. 4C where error bars are standard deviation between technical replicates shown as asterisks.

FIG. 24. Plasmid map and growth curves of IPTG induced lysis of SL1344-Holin. (A) Plasmid map of pRG1 modified to include the lacIq gene showing the Holin-Endolysin cassette, Ampicillin resistance gene, lac promoter, and pMB1 origin of replication. (B) Optical density was measured at 600 nm and normalized optical density is reported as absorbance at 600 nm minus media blank and each biological replicate normalized to the optical density at time zero. 1 mM IPTG was added for induction. Error bars indicate standard deviation of n=3 biological replicates.

FIG. 25. α-recA growth inhibition of SL1344. Optical density was measured at 600 nm in 50 μL cultures and normalized optical density is reported at 24 hours as absorbance at 600 nm minus media blank and each biological replicate normalized to the optical density reading at time zero. Overnight cultures of SL1344 were diluted 1:10,000 and treated with 10 μM addition of α-recA for 24 hours. Error bars indicate standard deviation of n=3 biological replicates.

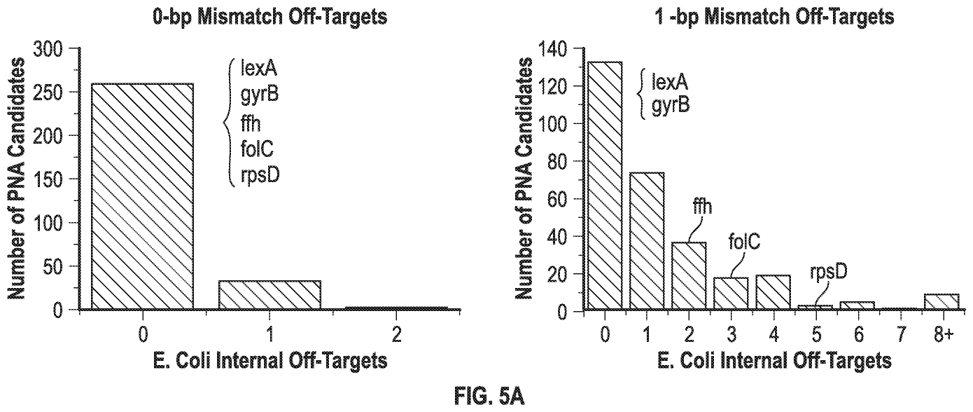

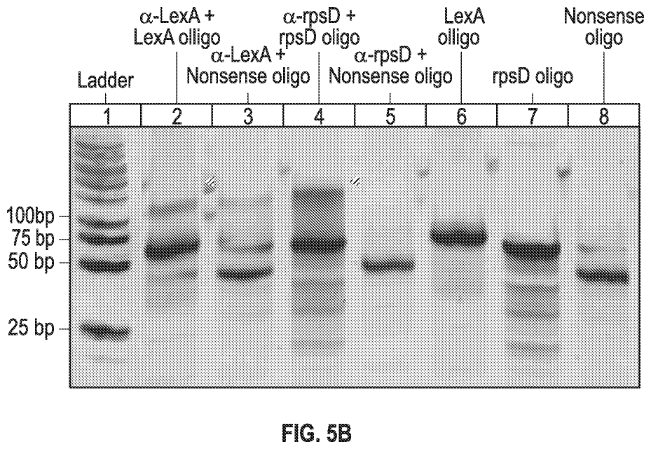

FIG. 26. Raw CFU/mL in double infection study. Following double infection experiments colony forming units were counted for both SL1344-mCherry (determined by florescence) and SL1344-Holin (FIG. 5B-C). Intracellular SL1344 colonies were enumerated by lysing with 30 μL of 0.1% Triton for 15 minutes at room temperature then diluted 1:10 by addition of 270 μL of DPBS. The lysate was serially diluted 1:10 in 100 μLs and 10 μL plated onto LB agar with 40 μg/mL of Streptomycin and 100 μg/mL Ampicillin. Error bars indicate standard deviation of technical replicates.

FIG. 27. Normalized mCherry/Holin treatment with IPTG. The same protocol for the double infection clearance using SL1344-Holin was used but during treatment 1 mM IPTG was added to induce lysis and release of the PNA instead of relying on leaky expression. Significant decrease in the normalized mCherry/Holin was seen for the condition with α-recA without IPTG (leaky expression, p=0.026) and with IPTG (p=0.006).

### MODE(S) FOR CARRYING OUT THE INVENTION(S)

The following detailed description is provided to aid those skilled in the art in practicing the various embodiments of the present disclosure, including all the methods, uses, compositions, etc., described herein. Even so, the following detailed description should not be construed to unduly limit the present disclosure, as modifications and variations in the embodiments herein discussed may be made by those of ordinary skill in the art without departing from the spirit or scope of the present discoveries.

Generally, the inventive technology includes the use of predictive homology to design novel broad-pathogen and gene-specific antisense RNA-inhibitors against clinical isolates of bacteria pathogens. Additional embodiment may include systems and methods to generate novel antibiotic compounds, and in particular novel antisense PNA-antibiotic compounds. In one embodiment, the invention includes the generation of novel antisense-PNA molecules directed to inhibit or downregulate non-traditional antibiotic pathways that may be effective at inhibiting cell growth in MDR clinical isolates. Additional preferred embodiments may include the use of novel antisense-PNA molecules as adjuvants and/or potentiators in combination with antibiotics, preferably directed to MDR bacteria. In certain embodiments, such antisense-PNA molecules may resensitize previously resistant bacterial strains to one or more traditional antibiotics.

In one specific embodiment, the invention may include methods, systems and compositions to treat MDR bacteria via the systematic design of gene/pathogen-specific PNA antisense therapeutics. In this preferred embodiment, PNA therapeutics may block the translation of a desired gene by binding to the transcriptional initiation region (TIR) for targeted growth inhibition or killing of the desired pathogen. Additional, embodiments of the invention may systems and methods for the novel introduction of antisense-PNA molecules to an intracellular environment through the use of bacterial Type-3 secretion system (T3SS) delivery method. Additional embodiments may include the incorporation introduction of antisense-PNA molecules to an intracellular environment through the use of bacterial (T3SS) delivery system in a non-pathogenic probiotic or endosymbiotic bacterium. In certain embodiments, such PNAs may also be directed to target essential genes in viral, fungal or parasitic pathogens.

As noted, in additional embodiments of the invention may systems and methods for the novel introduction of various therapeutic and/or diagnostic molecules to an intracellular environment through the use of bacterial secretion system, such as a Type-III (T3SS) or IV (T4SS) secretion system delivery method. Additional embodiments may include the introduction of therapeutic and/or diagnostic molecules, such as an antisense oligonucleotide, to an intracellular environment through the use of bacterial (T3SS) or (T4SS) delivery system in non-pathogenic probiotic bacteria. In certain embodiments, such PNAs or other antisense oligomers may also be directed to target essential genes in viruses among other pathogens. Additional non-limiting examples may include the delivery of one or more therapeutic and/or diagnostic molecules he use of bacterial (T3SS) or (T4SS) delivery system in non-pathogenic probiotic bacteria may be selected from the group consisting of: antibodies, therapeutic peptides, CRISPR, Zinc Fingers, or TALENS, as well as other small compounds or molecules, such as for example antibiotics.

Additional embodiments may include the introduction of antisense oligomers, such as a PNAs, as well as other therapeutic and/or diagnostic molecules to an intracellular environment through the use of bacterial (T3SS) or (T4SS) delivery system in a bacteroides, such as lactobactillus, L. bulgaricus, S. thermophillus, B. acidifaciens B. barnesiaes, B. caccae, B. caecicola, B. caecigallinarum, B. cellulosilyticus, B. cellulosolvens, B. clarus, B. coagulans, B. coprocola, B. coprophilus, B. coprosuis, B. distasonis, B. dorei, B. eggerthii, B. gracilis, B. faecichinchillae, B. faecis, B. finegoldii, B. fluxus, B. fragilis, B. galacturonicus, B. gallinaceum, B. gallinarum, B. goldsteinii, B. graminisolvens, B. helcogene, B. intestinalis, B. luti, B. massiliensis, B. melaninogenicus, B. nordii, B. oleiciplenus, B. oris, B. ovatus, B. paurosaccharolyticus, B. pectinophilus, B. plebeius, B. polypragmatus, B. propionicifaciens, B. putredinis, B. pyogenes, B. reticulotermitis, B. rodentium, B. salanitronis, B. salyersiae, B. sartorii, B. stercoris, B. stercoris, B. suis, B. tectus, B. thetaiotaomicron, B. uniformis, B. vulgatus, B. xylanisolvens, and B. xylanolyticusxylanolyticus.

In some embodiments, gene expression of a group of genes in a bacterium may be modulated by an antisense oligomer, such as an antisense PNA-based system. As used herein, PNAs may be DNA analogs in which the phosphate backbone has been replaced by (2-aminoethyl) glycine carboyl units that are linked to the nucleotide bases by the glycine amino nitrogen and methylene carbonyl linkers. The backbone is thus composed of peptide bonds linking the nucleobases. Because the PNA backbone is composed of peptide linkages, the PNA is typically referred to as having an amino-terminal and a carboxy-terminal end. However, a PNA can be also referred to as having a 5′ and a 3′ end in the conventional sense, with reference to the complementary nucleic acid sequence to which it specifically hybridizes. The sequence of a PNA molecule is described in conventional fashion as having nucleotides G, U, T, A, and C that correspond to the nucleotide sequence of the DNA molecule. Such polynucleotides can be synthesized, for example, using an automated DNA synthesizer. Typically, PNAs are synthesized using either Boc or Fmoc chemistry. PNAs and other polynucleotides can be chemically derivatized by methods known to those skilled in the art. For example, PNAs have amino and carboxy groups at the 5′ and 3′ ends, respectively, that can be further derivatized. Custom PNAs can also be synthesized and purchased commercially. Since PNA is structurally markedly different from DNA, PNA is very resistant to both proteases and nucleases, and is not recognized by the hepatic transporter(s) recognizing DNA.

Certain embodiments provide a PNA system for treating a bacterial infection and in particular a MDR bacterium. In accordance with these embodiments, a system can function to target traditional and/or non-traditional antibiotic pathways that may kill or arrest bacteria growth. In some embodiments, a PNA system for treating a bacteria infection includes at least one, at least two, at least three, or at least four or more PNAs, wherein each PNA includes a sequence of at least 5, and up to 20 or more nucleic acids capable of hybridizing to a target sequence of a single (i.e., unique) gene of the bacterium or other pathogen. In some embodiments, the antisense PNA molecules of the PNA system may be configured to target an essential gene of a pathogen, or a combination thereof. Exemplary groups of target genes are generally described above.

In some embodiments, an essential gene from a traditional, or non-traditional antibiotic target pathway may include essential genes related to metabolism, cell signaling, and stress response may be capable of killing or inhibiting MDR bacterial strains. The genetic DNA sequence, or RNA/mRNA sequences associated with antibiotic resistance that may be targeted with the novel PNAs-based system described herein can be any sequence that confers antibiotic resistance, or aids in development of resistance. In other embodiments, the RNA sequence associated with antibiotic resistance is a regulatory RNA affecting the antibiotic resistance of bacteria. In particular embodiments, the RNA sequence is a small RNA (sRNA) sequence. Regulatory RNAs can affect antibiotic resistance of bacteria by regulating, for example, RNA synthesis, protein synthesis, cell membrane integrity, membrane transporters, and cell wall turnover. These processes and mechanisms are known to be involved in antibiotic resistance of bacteria. Example target genes may include: folC (H2-folate synthetase); ffh (signal recognition particle); lexA (SOS response repressor; gyrB (gyrase subunit B); and/or rpsD (30S ribosomal protein). Naturally such embodiments are exemplary only and not intended to be limiting.

Certain preferred embodiments provide a PNA system for treating a bacterial infection, and in particular, a MDR bacterial infection. Common drug-resistant bacteria that may be treated by the systems, methods and compositions described herein may include, but not be limited to: carbapenem resistant Enterobacteriaceae Klebsiella pneumonia (CREKP), MDR tuberculosis (MDRTB), MDR Salmonella enterica, MDR Salmonella typhimurium (MDRST), methicillin-resistant Staphylococcus aureus (MRSA), vancomycin-resistant S. aureus (VRSA), extended spectrum β-lactamase Klebsiella pneumoniae (ESBL K. pneumoniae), vancomycin-resistant Enterococcus (VRE), carbapenem-resistant Enterobacteriaceae Escherichia coli (CRE E. coli), MDR Escherichia coli (MDR E. coli), New-Delhi metallo-β-lactamase producing Klebsiella pneumoniae (NDM-1 K. pneumoniae) and MDR Acinetobacter baumannii (MRAB).

It will be recognized by those of skill in the art that any of the DNA or mRNA sequences described above can be targeted by antisense inhibitors. Target sequences can be those of E. coli or the homologous gene or mRNA sequence in another target bacterium. Given the benefit of this disclosure, those of skill in the art will be able to identify a target sequence and design an antisense inhibitor oligomer to target the gene or mRNA sequence. Target sites on DNA or RNA (e.g. sRNA) associated with antibiotic resistance can be any site to which binding of an antisense oligomer will inhibit the function of the DNA or RNA sequence. Inhibition can be caused by steric interference resulting from an antisense oligomer binding the DNA RNA sequence, thereby preventing proper transcription of the DNA sequence or translation of the RNA sequence. Target sites, such as those identified as SEQ ID NOs. 1-14, on an essential gene associated with an antibiotic or other essential pathway, can be any site to which binding of an antisense oligomer will inhibit transcription of the gene.

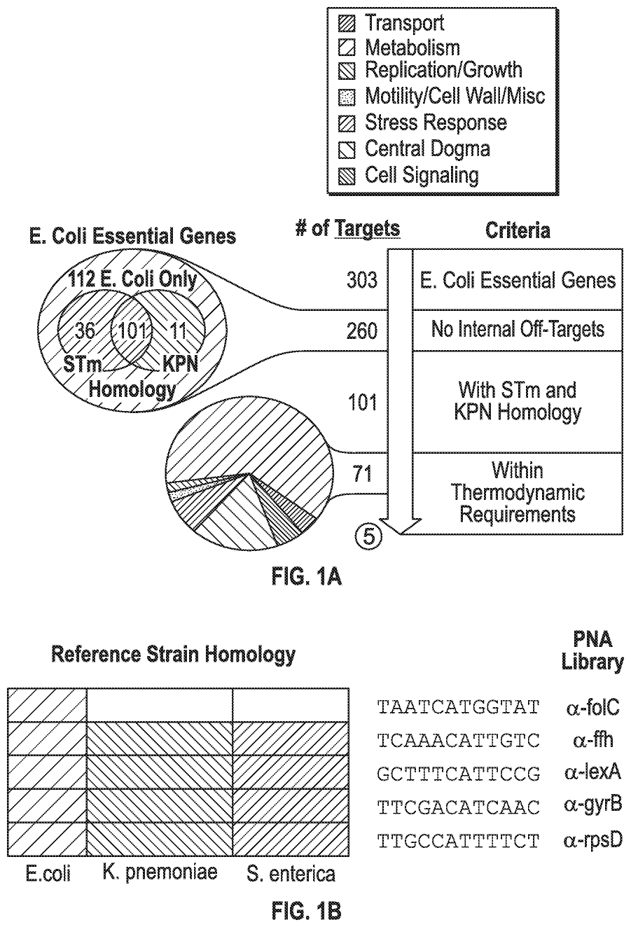

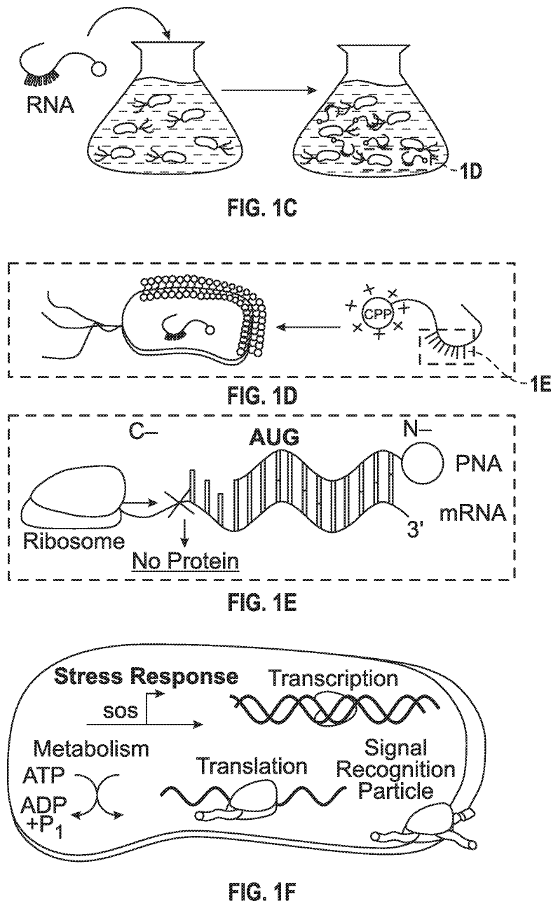

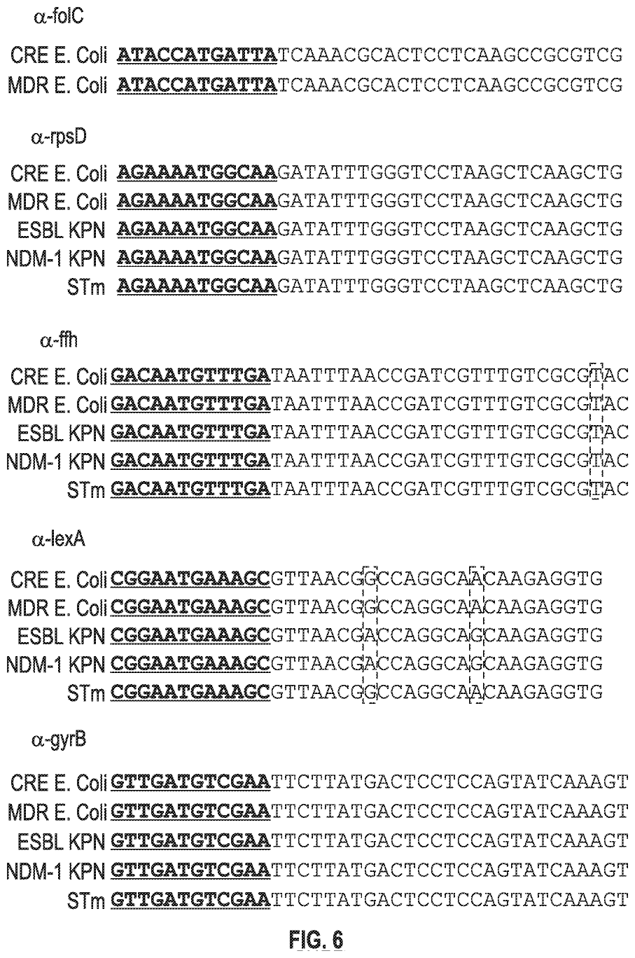

In certain embodiments, antisense sequences are designed to be centered on the start codon of a target gene. For example, as shown in FIGS. 1, 6 and Table 8, in one embodiment, antisense PNAs according to SEQ ID NO. 15-19 may be configured to target one or more pathogen genes, and more specifically in this embodiment α-folC, α-rspD, α-ffh, α-lexS and α-gyrB respectively. Again, as specifically demonstrated in FIG. 6, in this embodiment, such antisense PNA(s) may be configured to hybridize to the translation start codon region of the target gene. In this embodiment, the hybridization of the antisense PNA with the target gene start codon region may sterically hinder proper DNA transcription and thus inhibit expression of the target pathogen gene.

More particularly, target sites on an mRNA sequence associated with a traditional or non-traditional antibiotic resistance can be any site to which binding of an antisense oligomer will inhibit expression of the mRNA sequence. Protein functional sites on mRNA have been shown to be effective antisense sites for blocking ribosomal binding and migration. These include the ribosomal binding site (RBS) and translation start site (TSS), which are located in the 5′ untranslated region (UTR). In particular embodiments, the target site is a ribosomal binding site (RBS), a translational start site (TSS), or a YUNR motif Targeting an RBS inhibits ribosomal binding to the mRNA, thereby preventing translation of the mRNA, while targeting a TSS, prevents a bound ribosome from migrating past the start codon, thereby inhibiting translation. Targeting a 5′ YUNR motif results in a rate-limiting interaction between mRNA and ribosome, and can prevent ribosomal migration, thereby inhibiting mRNA translation.

An antisense oligomer can be complementary to a single target site or to two or more target sites. For example, an antisense oligomer can be complementary to any one of a TSS, RBS, or YUNR motif. However, an antisense oligomer can be complementary to two or more of the target sites. In particular embodiments, each individual antisense oligomer is complementary to a single target site. Wherein each individual antisense oligomer is complementary to a single target site, the antisense oligomer can be about 10-mers to about 20-mers in length. In certain embodiments, the antisense oligomer is about 12-mers in length. In certain embodiments, the antisense inhibitory oligomers are designed with the target sequence in the middle of the oligomer, with 3-5 nucleotides flanking the overlapping region. This provides for antisense oligomers with both high affinity and specificity. Wherein an individual antisense oligomer is complementary to two or more target sites, the antisense oligomer can be up to about 40-mers in length.

Because of the neutral PNA backbone, antisense PNAs tend to be hydrophobic. This hydrophobicity can impede uptake of the antisense PNA into bacterial cells. In order to overcome this, in a certain embodiment, a PNA can be linked to a cell penetrating peptide, for example as identified as amino acid sequence=SEQ ID NO. 23. Many cell penetrating peptides are known in the art, including but not limited to (KFF)₃K, penetratin, NLS, TAT, Arg(9), D-Arg(9), 10HC, cyLoP-1, Pep-1, and those cell penetrating peptides describe in U.S. Pat. No. 9,238,042 (Frederick et al., 2016, such specific CPP sequences being incorporated herein by reference), and can be linked to an antisense antibiotic oligomer, such as a PNA.

Antisense PNA sequences may be approximately twelve peptide nucleic acids long, centered on the mRNA target site or start codon. Antisense antibiotic oligomers may comprise the antisense sequence linked to a CPP such as a (KFF)₃K CPP attached at the N-terminus via an O-linker. Antisense antibiotic oligomers can comprise RNA or a nucleic acid analog such as PNA, morpholino, LNA, BNA, GNA, TNA, or 2′-O-methyl-substituted RNA.

In other embodiments, antisense inhibitor oligomers are designed against a target gene from one or more bacterial strain, and may be selected from the group of folC (SEQ ID NO. 1-2); rpsD (SEQ ID NO. 3-5); ffh (SEQ ID NO. 6-8); lexA (SEQ ID NO. 9-11); and gyrB (SEQ ID NO. 12-14). In certain embodiments, the antisense oligomers may comprise a CPP linked to the antisense inhibitor oligomer via an O-linker. Sequences and bacterial strains for such antisense inhibitor oligomers are provided in Table 1 and FIG. 1A. The antisense antibiotic oligomers used in accordance with this invention can be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents and knowledge of one of ordinary skill in the art.

A composition of the present disclosure can comprise one or more antisense oligomers, such as an antisense PNA. Where the composition comprises a single antisense oligomer, the antisense oligomer is capable of bactericidal effects, or has bacteriostatic effects. For example, a composition can comprise antisense PNA according to SEQ ID NO. 15, targeting α-folC of E. coli according to SEQ ID NOs. 1 or 2, and which binds to the translational start site of folC mRNA. By preventing translation of folC mRNA, this pathway may be inhibited and thereby induce a bactericidal effect. Alternately, an antisense PNA may have a bactericidal effect on its own. As demonstrated in the figures and description contain herein, α-rpsD significantly reduced the growth of all clinical isolates, with 99%, 98% and 99% growth inhibition across CRE E. coli, MDR E. coli and MDR STm respectively.

Also provided herein are compositions including 14 or more unique antisense PNA oligomers that target the sequences identified as SEQ ID NOs. 1-14. (See Table 1). Additionally, antisense PNA compositions identified in FIG. 1B are also included according to SEQ ID NOs. 15-19. It should be noted that the identification of any sequence herein, generally refers to the 12-mer sequence of each PNA which is shown N- to C-terminus. Additional sequences outside the target sequences may be variable while the sequences may also include a CPP or other linker. It should be noted that the start codon in each target sequence is italicized in the figures.

Such compositions can also have bactericidal or bacteriostatic effects, or can prevent emergence of antibiotic resistance. At least one antisense oligomer may be present in the composition at a pharmaceutically effective concentration or therapeutically effective amount. The pharmaceutically effective concentration of an antisense oligomer will depend on several factors, including but not limited to the oligomer's backbone composition, the affinity of the oligomer for its target, the specificity of the oligomer for its target, and the ability of the oligomer to enter the cell. In certain embodiments, a pharmaceutically effective concentration of an antisense oligomer is that concentration that prevents development of adaptive resistance. In particular embodiments, wherein the antisense oligomer is an antisense PNA.

As described above, the antisense antibiotic oligomers can be used in a synergistic combination with other known antimicrobial agents which may selected from the group consisting of: penicillins, cephalosporins, carbacephems, cephamycins, carbapenems, monobactams, aminoglycosides, glycopeptides, quinolones, tetracyclines, macrolides, and fluoroquinolones. Examples of antibiotic agents may be further selected from the group consisting of: Penicillin G (CAS Registry No.: 61-33-6); Methicillin (CAS Registry No.: 61-32-5); Nafcillin (CAS Registry No.: 147-52-4); Oxacillin (CAS Registry No.: 66-79-5); Cloxacillin (CAS Registry No.: 61-72-3); Dicloxacillin (CAS Registry No.; 3116-76-5); Ampicillin (CAS Registry No.: 69-53-4); Amoxicillin (CAS Registry No.: 26787-78-0); Ticarcillin (CAS Registry No.: 34787-01-4); Carbenicillin (CAS Registry No.: 4697-36-3); Mezlocillin (CAS Registry No.: 51481-65-3); Azlocillin (CAS Registry No.: 37091-66-0); Piperacillin (CAS Registry No.: 61477-96-1); Imipenem (CAS Registry No.: 74431-23-5); Aztreonam (CAS Registry No.: 78110-38-0); Cephalothin (CAS Registry No.: 153-61-7); Cefazolin (CAS Registry No.: 25953-19-9); Cefaclor (CAS Registry No.: 70356-03-5); Cefamandole formate sodium (CAS Registry No.: 42540-40-9); Cefoxitin (CAS Registry No.: 35607-66-0); Cefuroxime (CAS Registry No.: 55268-75-2); Cefonicid (CAS Registry No.: 61270-58-4); Cefinetazole (CAS Registry No.: 56796-20-4); Cefotetan (CAS Registry No.: 69712-56-7); Cefprozil (CAS Registry No.: 92665-29-7); Lincomycin (CAS Registry No.: 154-21-2); Linezolid (CAS Registry No.: 165800-03-3); Loracarbef (CAS Registry No.: 121961-22-6); Cefetamet (CAS Registry No.: 65052-63-3); Cefoperazone (CAS Registry No.: 62893-19-0); Cefotaxime (CAS Registry No.: 63527-52-6); Ceftizoxime (CAS Registry No.: 68401-81-0); Ceftriaxone (CAS Registry No.: 73384-59-5); Ceftazidime (CAS Registry No.: 72558-82-8); Cefepime (CAS Registry No.: 88040-23-7); Cefixime (CAS Registry No.: 79350-37-1); Cefpodoxime (CAS Registry No.: 80210-62-4); Cefsulodin (CAS Registry No.: 62587-73-9); Fleroxacin (CAS Registry No.: 79660-72-3); Nalidixic acid (CAS Registry No.: 389-08-2); Norfloxacin (CAS Registry No.: 70458-96-7); Ciprofloxacin (CAS Registry No.: 85721-33-1); Ofloxacin (CAS Registry No.: 82419-36-1); Enoxacin (CAS Registry No.: 74011-58-8); Lomefloxacin (CAS Registry No.: 98079-51-7); Cinoxacin (CAS Registry No.: 28657-80-9); Doxycycline (CAS Registry No.: 564-25-0); Minocycline (CAS Registry No.: 10118-90-8); Tetracycline (CAS Registry No.: 60-54-8); Amikacin (CAS Registry No.: 37517-28-5); Gentamicin (CAS Registry No.: 1403-66-3); Kanamycin (CAS Registry No.: 8063-07-8); Netilmicin (CAS Registry No.: 56391-56-1); Tobramycin (CAS Registry No.: 32986-56-4); Streptomycin (CAS Registry No.: 57-92-1); Azithromycin (CAS Registry No.: 83905-01-5); Clarithromycin (CAS Registry No.: 81103-11-9); Erythromycin (CAS Registry No.: 114-07-8); Erythromycin estolate (CAS Registry No.: 3521-62-8); Erythromycin ethyl succinate (CAS Registry No.: 41342-53-4); Erythromycin glucoheptonate (CAS Registry No.: 23067-13-2); Erythromycin lactobionate (CAS Registry No.: 3847-29-8); Erythromycin stearate (CAS Registry No.: 643-22-1); Vancomycin (CAS Registry No.: 1404-90-6); Teicoplanin (CAS Registry No.: 61036-64-4); Chloramphenicol (CAS Registry No.: 56-75-7); Clindamycin (CAS Registry No.: 18323-44-9); Trimethoprim (CAS Registry No.: 738-70-5); Sulfamethoxazole (CAS Registry No.: 723-46-6); Nitrofurantoin (CAS Registry No.: 67-20-9); Rifampin (CAS Registry No.: 13292-46-1); Mupirocin (CAS Registry No.: 12650-69-0); Metronidazole (CAS Registry No.: 443-48-1); Cephalexin (CAS Registry No.: 15686-71-2); Roxithromycin (CAS Registry No.: 80214-83-1); Co-amoxiclavuanate; combinations of Piperacillin and Tazobactam; and their various salts, acids, bases, and other derivatives.

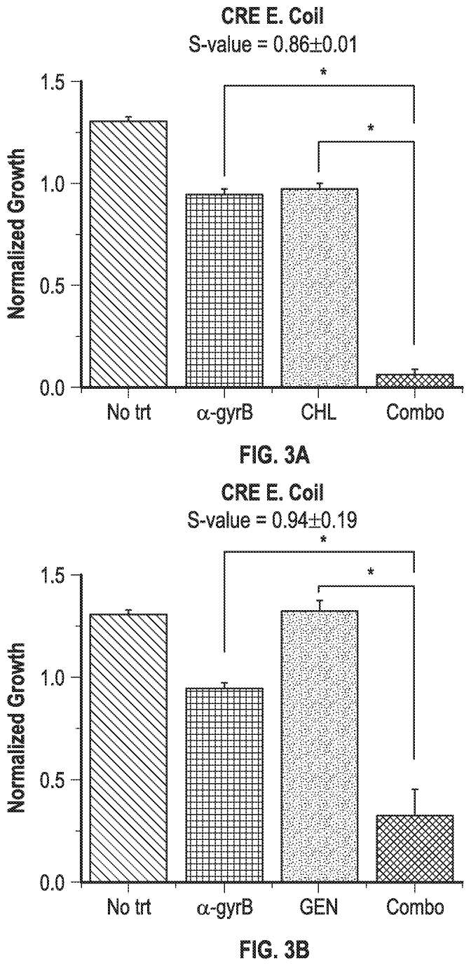

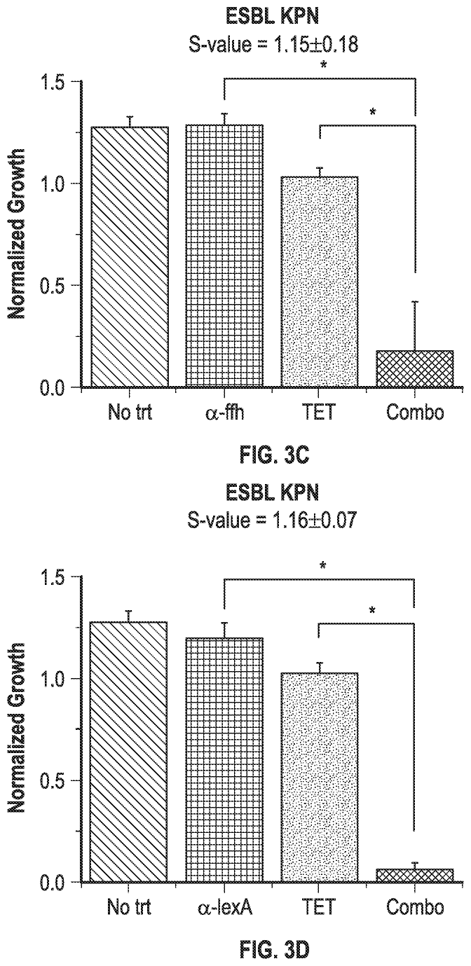

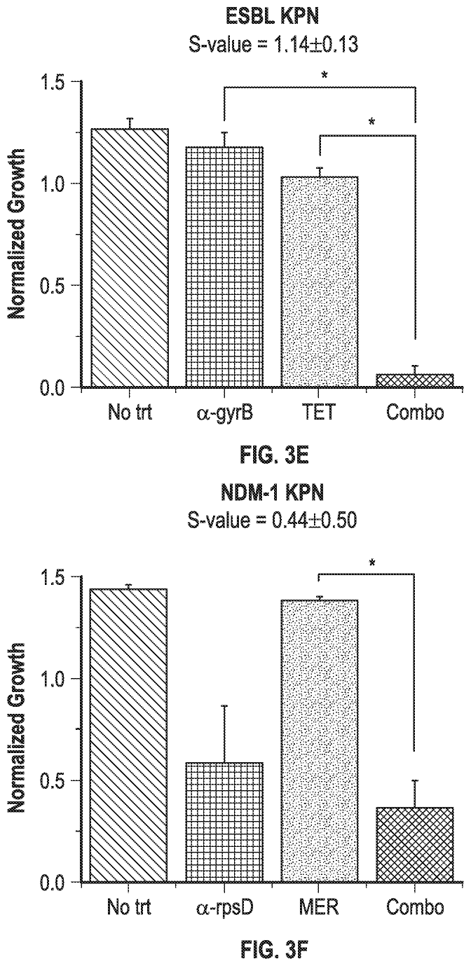

As generally shown in FIG. 3, in certain embodiments, the composition is a pharmaceutical composition. The compositions may routinely contain pharmaceutically acceptable concentrations of salt, buffering agents, preservatives and various compatible carriers or diluents. The proportion and identity of the pharmaceutically acceptable diluent is determined by chosen a route of administration, compatibility with the antisense oligomers of the composition, and standard pharmaceutical practice. Generally, the pharmaceutical composition will be formulated with components that will not significantly impair the biological activities of the antisense oligomers. The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the compositions are prepared uniformly and intimately, bringing into association the active ingredients with liquid carriers or finely divided solid carriers or both, and then, if necessary, shaping the product. One of skill in the art will recognize that formulations are routinely designed according to their intended use, i.e. route of administration.

Wherein the composition comprises one or more PNAs having bactericidal or bacteriostatic effects, a subject can be treated for bacterial infection by administering an appropriate dose of the composition. Optionally, such compositions can comprise one or more conventional antibiotics. Compositions described herein can be administered similarly to currently available antibiotics, including but not limited to oral administration, nasal administration, intravenous administration, intramuscular administration, intraperitoneal administration, topical administration, local delivery methods, and in feed and water supplies.

Subjects to be treated for a bacterial infection can be selected from the group of: human; feed animals including but not limited to cattle, swine, poultry, goat, and sheep; companion animals including but not limited to dog, cat, rodent, bird, and reptile; and laboratory animals. Subjects to be re-sensitized to an antibiotic can be those who have shown resistance to an antibiotic, or to whom an antibiotic is to be given where there is common drug resistance to the antibiotic. A composition described herein can also be provided to a subject in order to prevent or delay development of antibiotic resistance.

Methods are provided for treating a bacterial infection in a subject in need thereof. Subjects to be treated for a bacterial infection are administered a composition described herein, thereby treating the bacterial infection. In certain embodiments, the composition used for treating a bacterial infection targets at least one mRNA sequence that encodes a protein essential for bacterial homeostasis. In certain embodiments, the composition does not comprise a conventional antibiotic, while in other embodiments, the composition does comprise at least one conventional antibiotic. In yet other embodiments, a composition comprising at least one PNA capable of affecting translation of at least one drug resistance-associated enzyme or protein is administered to the subject first, followed by administration of an antibiotic. In preferred embodiments, the subject is human. In some embodiments, a PNA may be delivered through a T3SS of a bacterium, such as a probiotic, and may further be used in conjunction with the administration of an antibiotic.

Examples of suitable probiotic microorganisms include yeasts such as Saccharomyces, Debaromyces, Candida, Pichia and Torulopsis, molds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus. Specific examples of suitable probiotic microorganisms are: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus reuteri, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, and Staphylococcus xylosus.

The formulation of therapeutic compositions and their subsequent administration (dosing) is believed to be within the skill of those in the art. Optimal dosing schedules can be calculated from measurements of drug accumulation in the body of a subject. Persons of ordinary skill can easily determine optimum dosages, dosing methodologies and repetition rates. Optimum dosages may vary depending on the relative potency of individual PNAs, and can generally be estimated based on EC₅₀s found to be effective in in vitro and in vivo animal models. In general, dosage is from 0.01 μg to 100 μg per kg of body weight, and may be given once or more daily, weekly, or monthly. Persons of ordinary skill in the art can easily estimate repetition rates for dosing based on measured residence times and concentrations of the antisense antibiotic oligomers in bodily fluids or tissues. Following successful treatment, it may be desirable to have the subject undergo maintenance therapy to prevent the recurrence of the disease state, wherein one or more PNAs is administered in maintenance doses, ranging from 0.01 μg to 100 μg per kg of body weight. In certain embodiments, a patient is treated with a dosage of one or more PNAs that is at least about 1, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100 mg/kg body weight.

As noted above, in one embodiment of the invention, the present inventors present a novel approach which takes advantage of an exemplary Type III secretion system (T3SS) to deliver PNAs (see generally results in FIG. 4A). The T3SS is a molecular machine used by many gram-negative bacterial pathogens including pathogens Shigella, Yersinia, Salmonella, and Pseudomonas, to inject proteins, known as effectors, directly into eukaryotic host cells. Specifically, Salmonella has been shown to enter host cells such as macrophages and osteoblasts through either phagocytosis or use of the Type III secretion system encoded by the Salmonella pathogenicity island-1 proteins. This infection can lead to the death of the host cell as well as further infection of other cell types in the body and systemic infection due to the proliferation of bacteria within the Salmonella-containing vacuole, through the expression of proteins on the Salmonella pathogenicity island-2.

In one embodiment, one or more antisense PNAs, which may further be coupled with a CPP, may be introduced to a bacterium having a T3SS. In a preferred embodiment, one or more PNAs may be introduced and taken up by a bacterium having a T3SS which may subsequently be introduced to an infected cell or tissue. The PNAs may act according to its design and inhibit expression of one or more target genes, initiating a bacteriostatic or bactericidal effect. In a preferred embodiment, bacteria, having a T3SS may include bacteria that are a naturally occurring, or a probiotic or endosymbiont with the host that has a T3SS. As used herein, the term “probiotic” generally refers to bacteria that may colonize a target host for sufficient time to deliver one or more PNAs to said host. In this embodiment, a probiotic may be introduced to, and uptake, one or more PNAs which may then be administered to the host allowing intracellular transport and delivery to the targeted PNAs to the host cell. Administration of an antibiotic may also be coupled with the aforementioned treatment.

To address the issue of poor PNA transport properties, in certain embodiment the present inventors may repurpose a bacterial T3SS. As noted above, T3SS are molecular machines used by many Gram-negative bacterial pathogens including Shigella, Yersinia, Salmonella and Pseudomonas, to inject proteins, known as effectors, directly into eukaryotic host cells. These proteins manipulate host signal transduction pathways and cellular processes to the pathogen's advantage. Here, the present inventors may re-purpose the intrinsic T3SS in, for example, Shigella flexneri or Salmonella typhimurium for developing a delivery vehicle for the designed antisense molecules, such as antisense PNAs, which may further be coupled with a CPP. Using a probiotic may also allow the present inventors to switch PNA payload in a facile manner. One improvement of this platform is the ability to reduce the need for approval from regulatory bodies such as FDA for each antisense molecule, such as a PNA, used in the invention.

As noted above, the present inventors have demonstrated that Salmonella (which express T3SS) pretreated with PNAs is capable of carrying the PNA into epithelial HeLa and macrophage cell. As such, in certain embodiments, the invention includes a system in which the T3SS functions will be introduced in a non-pathogenic strain of bacteria, such as E. coli Nissle 1917, a probiotic strain that is easily culturable and has been tested in humans for treatment of irritable bowel syndrome. E. coli Nissle 1917 encoding the T3SS may be incubated with PNA-(KFF)₃K molecules for 3-6 hours to ensure entry of PNA molecules into cells. Due to lack of proteases or nucleases that are known to degrade PNAs, once imported into E. coli the PNA-CPP may be stable inside the cell. The amount of PNA-(KFF)₃K uptake may be quantified by measuring fluorescence of a FITC labeled PNA molecule (FITC-(KFF)₃K-PNA).

In a preferred embodiment, the present inventors may adopt the T3SS from the pathogen Shigella flexneri and modify the system to generate a protein delivery system composed of three modules: (1) the ˜31 kb long minimal DNA sequence that encodes delivery apparatus of a functional T3SS from S. flexneri, and (2) the transcriptional activator VirB to induce expression of the T3SS, and (iii) a kill switch circuit under the control of the Ipac promoter. Ipac is the native Shigella T3SS encoded translocator protein that gets activated once Shigella invades a mammalian cell to activate cell lysis once E. coli enters mammalian cells.

In one embodiment, the present inventors may adopt such a kill switch design based on the expression of holin and antiholin (Biobricks part Ba_K112808) system. Holin is a protein that forms pores in cell membranes. Anti-holin forms a dimer with holin, which is inactive. The Ipac controlled expression of Holin will activate pore formation once the T3SS encoding E. coli Nissle 1917 has successfully infected a mammalian cell. Once pores are formed by holin, lysozyme is expected to access the periplasmic space and degrade the cell wall, causing cell lysis and likely release of PNA-CPP molecules, which will eventually target the infecting pathogen. Using the probiotic strategy PNAs will no longer be in the blood stream, therefore the present inventors may eliminate the issue of low bioavailability, as well as, transport into unintended host cells will be prevented, thereby addressing the toxicity issue. An alternative embodiment would be to use the T3SS from Salmonella or broad-host Type IV secretion system such as that encoded by the RK2 plasmid system to deliver PNA or other antisense molecules.

The terminology used herein is for describing embodiments and is not intended to be limiting. As used herein, the singular forms “a,” “and” and “the” include plural referents, unless the content and context clearly dictate otherwise. Thus, for example, a reference to “a target gene” may include a combination of two or more such target genes. Unless defined otherwise, all scientific and technical terms are to be understood as having the same meaning as commonly used in the art to which they pertain.

The present invention includes all compositions and methods relying on correlations between the reported PNAs and the bacterial effects host. Such methods include methods for determining whether a patient is predicted to respond to administration of PNA and/or antibiotic therapy, as well as methods for assessing the efficacy of PNA and/or antibiotic therapy, therapy. Such diagnostic information may be used to more effectively treat or kill, for example, MDR bacteria, while reducing or ameliorating further drug resistance. This diagnostic activity may be done in vivo, or ex vivo.

Further included are methods for improving the efficacy of a therapy for bacterial infection by administering to a subject a therapeutically effective amount of an agent that alters the activity or expression of a target gene in a pathogenic bacteria. In this context, the term “effective” is to be understood broadly to include reducing or alleviating the signs or symptoms of a bacterial infection, improving the clinical course of a bacterial infection, enhancing killing of bacteria, or reducing any other objective or subjective indicia of a bacterial infection. Different drugs, doses and delivery routes can be evaluated by performing the method using different drug administration conditions. The markers may also be used as pharmaceutical compositions or in kits.

The present invention includes all compositions and methods relying on correlations between the reported PNAs and the antibiotic sensitivity of a bacteria. Such methods include methods for determining whether a patient is predicted to respond to administration of PNA, or PNA+antibiotic therapy, as well as methods for assessing the efficacy of such therapies generally. In one embodiment, the action, whether alone or synergistically with another antibiotic, or select PNAs may be measured and/or characterized such that it may be a predictor of clinical outcome in patients prior to receiving antibiotic therapy, or patients that have severe infection possibly resulting in resistance to traditional antibiotics. Further included, are methods for improving the efficacy of an antibiotic therapy by administering to a subject a therapeutically effective amount of a PNA that alters the activity or expression of one or more target genes and/or that works synergistically to improve the bacteriostatic or bactericidal effects of an antibiotic.

Further included are methods for improving the efficacy of a cancer therapy by administering to a subject a therapeutically effective amount of one or more PNAs, or a carrier bacteria expressing a PNA, such a probiotic and the like. In this context, the term “effective” or “effective amount” or “therapeutically effective amount” is to be understood broadly to include reducing a bacterial population through a bactericidal or bacteria static mechanisms, improving the clinical course of the disease, or reducing any other objective or subjective indicia of the disease, or inducing an observable effect. Different doses and delivery routes can be evaluated by performing the method using different administration conditions. Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

As used herein, “antisense oligomers” or “antisense oligonucleotide” means any antisense molecule that may modulate the expression of one or more genes. Examples may include antisense PNAs, antisense RNA. This terms also encompasses RNA or DNA oligomers such as interfering RNA molecules, such as dsRNA, dsDNA, mRNA, siRNA, or hpRNA as well as locked nucleic acids, BNA, polypeptides and other oligomers and the like.

The term “PNA” “peptide nucleic acid”, as used herein, refers to a DNA mimic with a neutral polyamide backbone on which the nucleic acid bases are attached in the same manner as they are to the phosphate backbone of DNA. PNAs can be synthesized routinely by standard peptide chemistry and can be labeled with biotin or various other labels as either part of the solid phase synthesis, or following cleavage from the resin. See, e.g., Chollet et al., Nucleic Acids Research, Vol. 13, No. 5, pp. 1529-1541 (1985).

As used herein, the term “gene” or “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example, a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

The term “target sequence,” may mean a nucleotide sequence, such as a DNA sequence, or a mRNA sequence, that may be complementary to an antisense molecules, and preferably an antisense peptide nucleic acid.

The term “including” is used herein to mean, and is used interchangeably with, the phrase “including but not limited to.” The term “or” is used herein to mean, and is used interchangeably with, the term “and/or,” unless context clearly indicates otherwise.

The term “homologous” or “sequence identity” as used herein means a nucleic acid (or fragment thereof), including morpholino nucleic acids, or a protein (or a fragment thereof) having a degree of homology to the corresponding natural reference nucleic acid or protein that may be in excess of 70%, or in excess of 80%, or in excess of 85%, or in excess of 90%, or in excess of 91%, or in excess of 92%, or in excess of 93%, or in excess of 94%, or in excess of 95%, or in excess of 96%, or in excess of 97%, or in excess of 98%, or in excess of 99%). For example, in regard to peptides or polypeptides, the percentage of homology or identity as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage of homology as described above is calculated as the percentage of the components found in the smaller of the two sequences that may also be found in the larger of the two sequences (with the introduction of gaps), with a component being defined as a sequence of four contiguous amino acids. Also included as substantially homologous is any protein product which may be isolated by virtue of cross reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

In another embodiment, the invention includes PNAs that have substantial sequence similarity to the PNAs described herein. Two PNAs have “substantial sequence identity” when there is at least about 70% sequence identity, at least about 80% sequence identity, at least about 90% sequence identity, at least about 95% sequence identity or at least 99% sequence identity. Reference to one PNA refers explicitly to a homolog having substantial sequence similarity.

In yet another embodiment, the PNA comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

Optionally, the therapeutic methods of the present invention may be combined with other antibiotic therapies. For example, when two or more different treatments are combined, the treatments may work synergistically and allow reduction of dosage of each of the treatments, thereby reducing the detrimental side effects exerted by each compound at higher dosages. As such, in certain embodiments, the use of PNAs in conjunction with antibiotics may allow a reduction in the dosage of said antibiotic. This embodiment not only saves time and money, but also reduces the emergence of bacterial antibiotic resistance.

As used herein, the term “endogenous” refers to any material from or produced inside an organism, cell, tissue or system.

As used herein, the term “exogenous” refers to any material introduced from or produced outside an organism, cell, tissue or system.

As used herein, the terms “inhibit” and “inhibition” means to reduce a molecule, a reaction, an interaction, a gene, an mRNA, and/or a protein's expression, stability, function or activity by a measurable amount, or to prevent such entirely. “Inhibitors” are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, the terms “complementary” or “complement” also refer to a nucleic acid comprising a sequence of consecutive nucleobases or semiconsecutive nucleobases (e.g., one or more nucleobase moieties are not present in the molecule) capable of hybridizing to another nucleic acid strand or duplex even if less than all the nucleobases do not base pair with a counterpart nucleobase. In certain embodiments, a “complementary” nucleic acid comprises a sequence in which about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%), about 98%), about 99%, or about 100%, and any range derivable therein, of the nucleobase sequence is capable of base-pairing with a single or double stranded nucleic acid molecule during hybridization. In certain embodiments, the term “complementary” refers to a nucleic acid that may hybridize to another nucleic acid strand or duplex in stringent conditions, as would be understood by one of ordinary skill in the art.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Rational Design of PNAs Across Multiple Pathogens Against Non-Traditional Antibiotic Pathways and Novel Genes to Inhibit Multi-Drug Resistant Bacteria

The present inventors developed a bioinformatics toolbox to design gene-specific antisense-PNA RNA-inhibitors against five clinical isolates of Enterobacteriaceae, including carbapenem-resistant E. coli and extended spectrum β-lactamase-producing K. pneumoniae. Our toolbox uses predictive homology to design PNAs against a total of 303 well-characterized essential genes in E. coli as identified in the Database of Essential Genes. Unique 12-mer PNAs were designed to complement the start codon (AUG) of essential genes of interest in the middle of the oligomer, with 4-5 nucleotides flanking the start codon. These were refined to 260 candidates that did not show any potential off-targets at the start codon (STC) of an untargeted gene in E. coli MG1655 (FIG. 1A, FIG. 5A). The present inventors did not consider non-start site off-target genes because PNA has been shown to have the highest efficacy when the antisense molecule is a perfect match centered on the start codon. Additionally, partial sequence-mismatch effects greater than 1 base pair were not considered since PNA have been shown not to tolerate 2 or more base pair mismatches. The present inventors narrowed the list to 101 PNA candidates that showed homology in two other reference strains of our clinical isolates: K. pneumoniae MGH 78578 and Salmonella enterica serovar typhimurium SL1344. Finally, the present inventors refined the list to 71 total candidates that did not demonstrate self-complementation and were less likely to have solubility issues under experimental conditions as generally described below.

The present inventors sampled five PNA molecules, a small fraction of the design space of 71 PNA candidates, with significant therapeutic success (>50%) in comparison to current small molecule screening which achieves about <0.0001% success. These PNA molecules target non-traditional pathways including H2-folate synthetase (folC) in folate biosynthesis, the signal recognition particle protein gene (ffh) which is essential for protein translocation, and the gene for SOS response repressor protein (lexA) (FIG. 1B, F, Table 1). The present inventors also designed PNAs against traditional antibiotic targets similar to fluoroquinolones and tetracycline/aminoglycosides: gyrase subunit B (gyrB) and 30S ribosomal protein S4 (rpsD) respectively. During PNA design, a sequence expected to target only E. coli strains (α-folC) was included to assess the ability to rationally design species-selective antisense molecules. The remaining four antisense molecules, targeting rpsD (α-rpsD), ffh (α-ffh), lexA (α-lexA), and gyrB (α-gyrB), were designed for homology against E. coli, K. pneumoniae, and S. enterica.

The present inventors analyzed the PNAs for off-targets within the start codon region of genes in S. enterica and K. pneumoniae when allowing for zero and single base pair mismatches. All PNAs had no zero basepair mismatch off-targets except for α-rpsD which showed one off-target in Salmonella (FIG. 5, Tables 2 and 3). When allowing for single base pair mismatches α-lexA, α-gyrB, α-ffh, α-folC, and α-rpsD showed zero, zero, 2, 3 and 5 off-targets respectively (Table 2, FIG. 5A-B). In addition to computationally predicted specificity, the PNAs with the least and the most predicted off-targets, α-lexA, and α-rpsD respectively, both showed specificity to their targets by electrophoretic mobility shift assay (FIG. 5B, Table 3). Each 12-mer PNA was conjugated to positively charged (KFF)₃K cell penetrating peptide (CPP) via an AEEA-linker to increase transport across the membranes of gram-negative bacteria including E. coli, K. pneumoniae, and S. enterica (FIG. 1C-E). The linker has been shown to reduce steric interference between the PNA and CPP during target binding. This allowed us to add PNA exogenously to culture without the need for specific transport or expression from a plasmid.

### Example 2: Antisense Antibiotics Inhibit Growth of Clinical Isolates

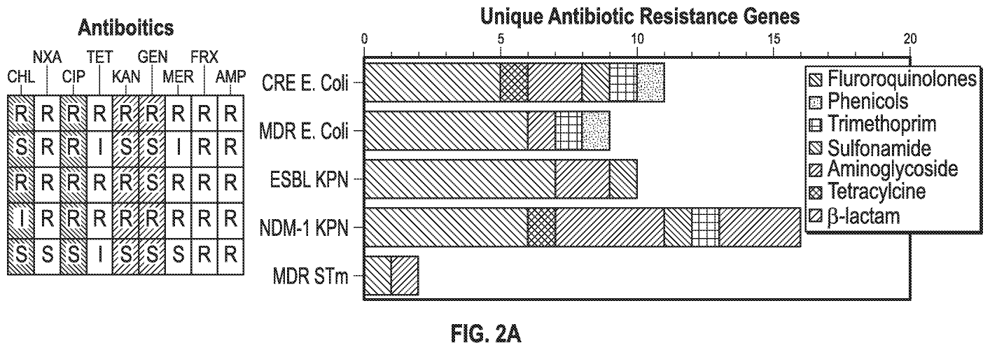

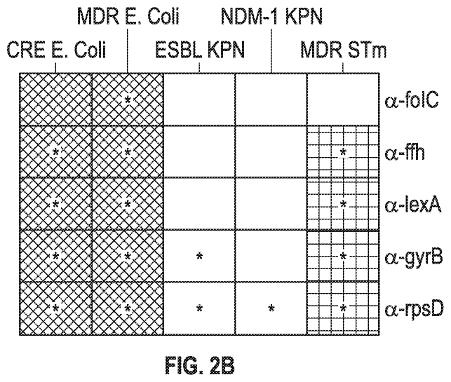

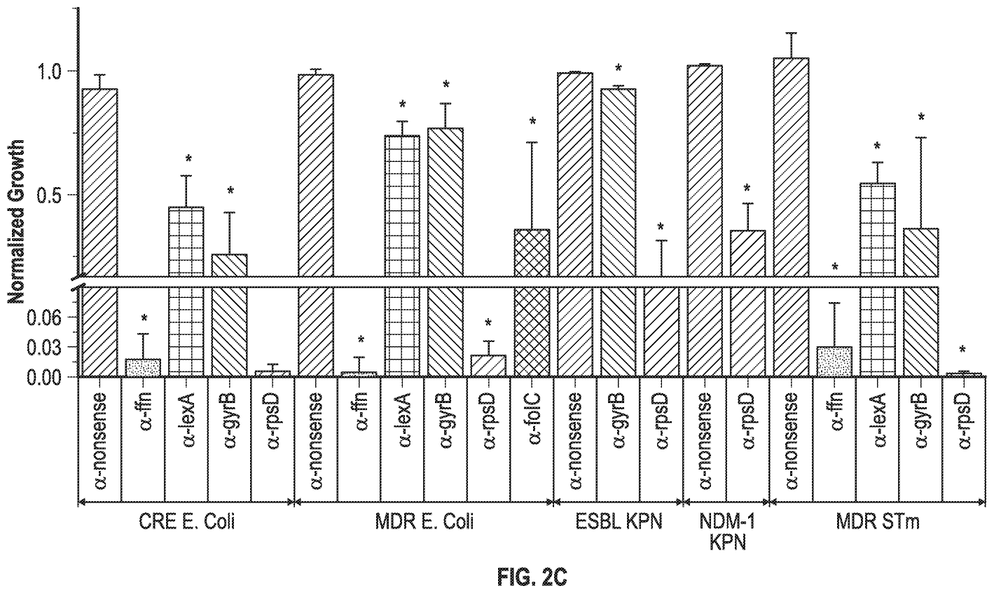

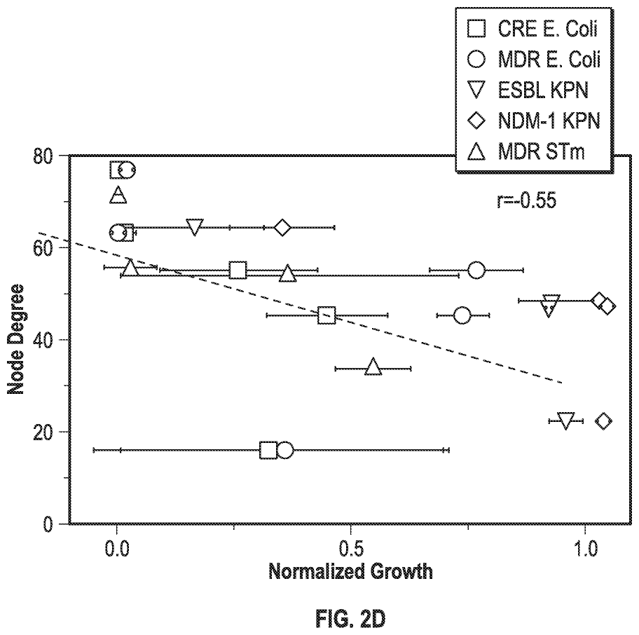

The PNAs were used to treat five randomly selected highly-resistant clinical isolates of Enterobacteriaceae including a carbapenem-resistant Enterobacteriaceae (CRE) E. coli, a multidrug-resistant (MDR) E. coli, an extended spectrum β-lactamase (ESBL)-producing K. pneumoniae (KPN), a New Delhi Metallo β-lactamase 1 (NDM-1) KPN, and an isolate of MDR S. enterica which was shown to be serovar typhimurium (STm). Phenotypic antibiotic resistance characterization of the clinical isolates was performed to determine “sensitive” (S), “intermediate” (I), and “resistant” (R) phenotypes using the 2016-2017 Clinical & Laboratory Standards Institute (CLSI) sensitive/resistant breakpoint values (Table 4). The present inventors screened nine antibiotics of varied mechanisms and classes including penicillins (ampicillin), cephalosporins (ceftriaxone), carbapenems (meropenem), aminoglycosides (gentamicin and kanamycin), tetracyclines (tetracycline), fluoroquinolones (ciprofloxacin), quinolones (nalidixic acid), and phenicols (chloramphenicol) (FIG. 2A (left panel)). All isolates were found to have resistance to two or more antibiotics, and in the extreme case, CRE E. coli showed resistance to all nine antibiotics tested. All strains were resistant to ampicillin and ceftriaxone.

In addition to antibiotic screening, the clinical isolates' genotypic resistance profile was screened using genome sequencing (FIG. 2A (right panel), Table 5). It was found that all of the clinical strains had at least two unique antibiotic resistance genes, and at least one β-lactamase gene, which confers resistance to varied β-lactam antibiotics. The number of unique antibiotic resistance genes identified in CRE E. coli, MDR E. coli, ESBL KPN, NDM-1 KPN, and MDR STm were 11, 9, 10, 16, and two, respectively. The resistance genes identified were cross-referenced with the phenotypic antibiotic resistance results and indicated a correlation between the presence of resistance genes identified and the resistance profile of the clinical isolate (Table 6). Through sequence analysis, the present inventors confirmed the presence of the antisense target sequences and found no potential sequence off targets in start codon regions in the clinical isolates, except one for α-rpsD (FIG. 6, Table 2).

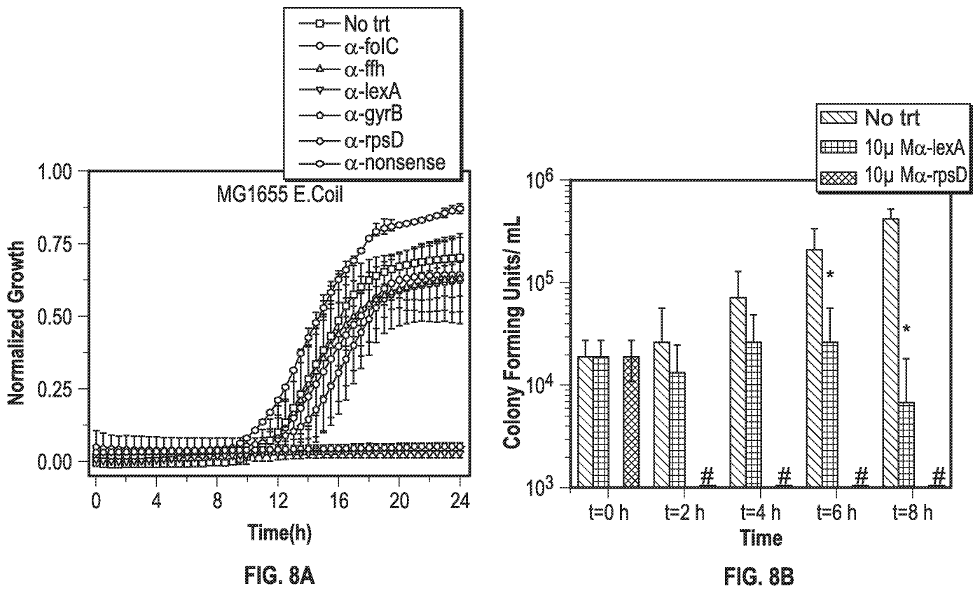

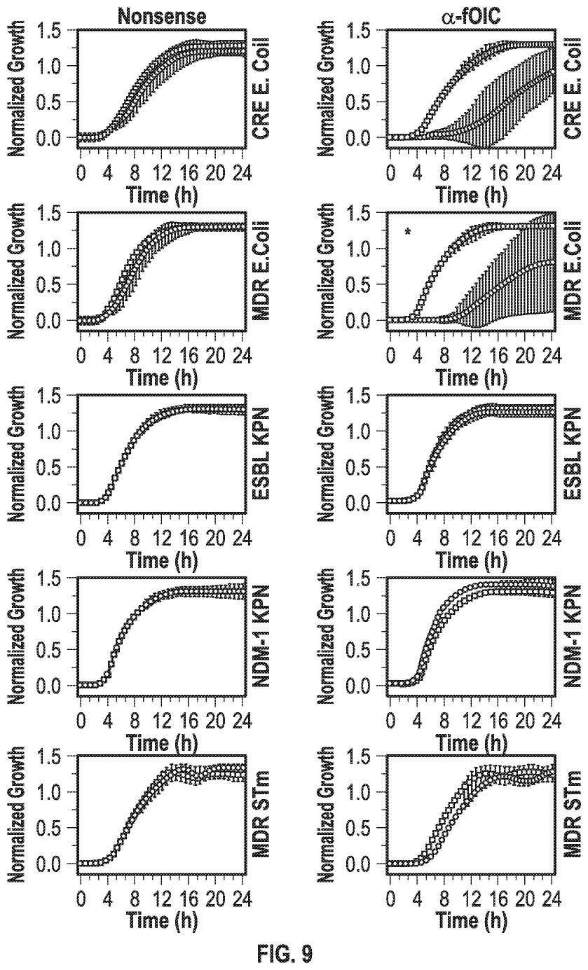

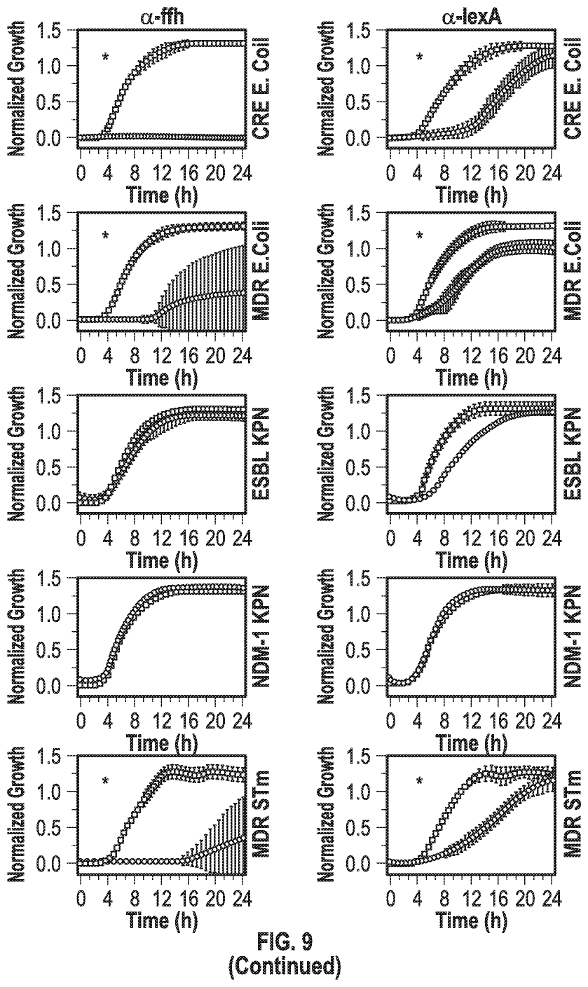

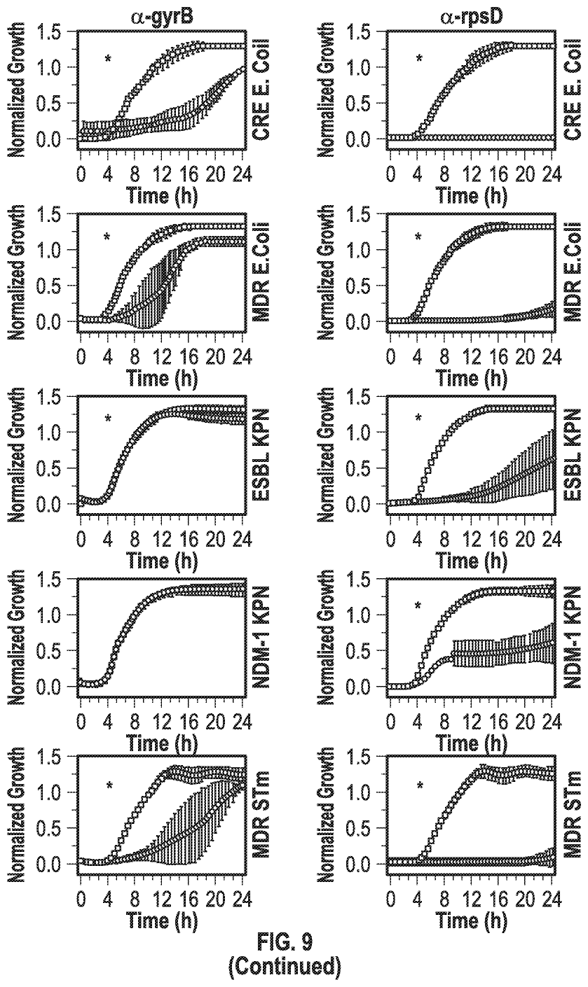

Despite significant resistance to traditional antibiotics, strong growth inhibition of clinical isolates was observed with PNA treatment (FIG. 2C). Testing of PNA molecules on a lab strain of E. coli, MG1655, showed that most PNAs were effective at a dose of 10 μM (FIG. 7, 8A-B). All further PNA testing was used at this concentration, including a control PNA molecule that targets a non-existent sequence in the select isolates and shows no growth inhibition, confirming no toxicity due to PNA or CPP (FIG. 2C, FIGS. 8A and 9). At this concentration 4 out of 5, 5 out of 5, 2 out of 4, 1 out of 4, and 4 out of 4 of all PNAs predicted to share homology with the isolates were successful in inhibiting growth of CRE E. coli, MDR E. coli, ESBL KPN, NDM-1 KPN, and MDR STm respectively (FIG. 2B,C and 9). The most effective PNA, α-rpsD, showed greater than 98% growth inhibition in CRE E. coli [99±0.4%], MDR E. coli [98±0.7%], and MDR STm [99±0.1%]. α-rpsD also caused 82%±8.8% and 65±7.1% growth inhibition in ESBL KPN and NDM-1 KPN respectively. α-gyrB showed significant growth inhibition in CRE E. coli [71±12%], MDR E. coli [36±14.2%], ESBL KPN [5±1.4%], and MDR STm [74±15.4%]. α-ffh showed greater than 98% growth inhibition in CRE E. coli [98±1.5%], MDR E. coli [99±0.9%], and MDR STm [99±0.8%]. Finally, α-lexA showed greater than 50% inhibition in CRE E. coli [51±8.1%] and MDR STm [62±5.4%]. Given that the PNAs were designed against non-pathogenic, drug-sensitive genomes and based on only E. coli's set of essential genes, the number of significantly inhibited isolates was a remarkable 73% of treatments. Out of these nearly 54% significantly reduced growth by greater than 50%, and nearly 27% of the treatments lead to more than 97% growth inhibition.

The present inventors next sought to further understand why certain PNA therapies were more effective than others through analysis of mRNA concentration, mRNA secondary structure, and protein interaction networks. Surprisingly, the most effective PNAs targeted RNAs with the highest intracellular mRNA concentration, producing a strong exponential decay relationship between the PNA efficacy and RNA levels (R²=0.86) (FIG. 15). The secondary structure of the target mRNA bore no relevance on PNA efficacy (FIG. 16-20). A network of all known protein interactions of each PNA target was constructed using the STRING database (FIG. 21) and observed a significant negative correlation between the average number of protein-protein partners and the corresponding PNA's growth inhibition of clinical isolates (Pearson correlation coefficient=−0.55, p=0.008, FIG. 2D). The present inventors identified specific cellular pathways (defined by the Kyoto Encyclopedia of Genes and Genomes) that are significantly over-represented within each of these networks, indicating the biological processes that were particularly disrupted by PNA therapy (FIG. 22). These data suggest that that the more interconnected a target gene's network of protein-protein partners is the more efficacious the PNA monotherapy was in preventing MDR growth.

### Example 3: Antisense-PNA Acts as a Potentiator and Adjuvant with Small-Molecule Traditional Antibiotics in MDR Bacteria

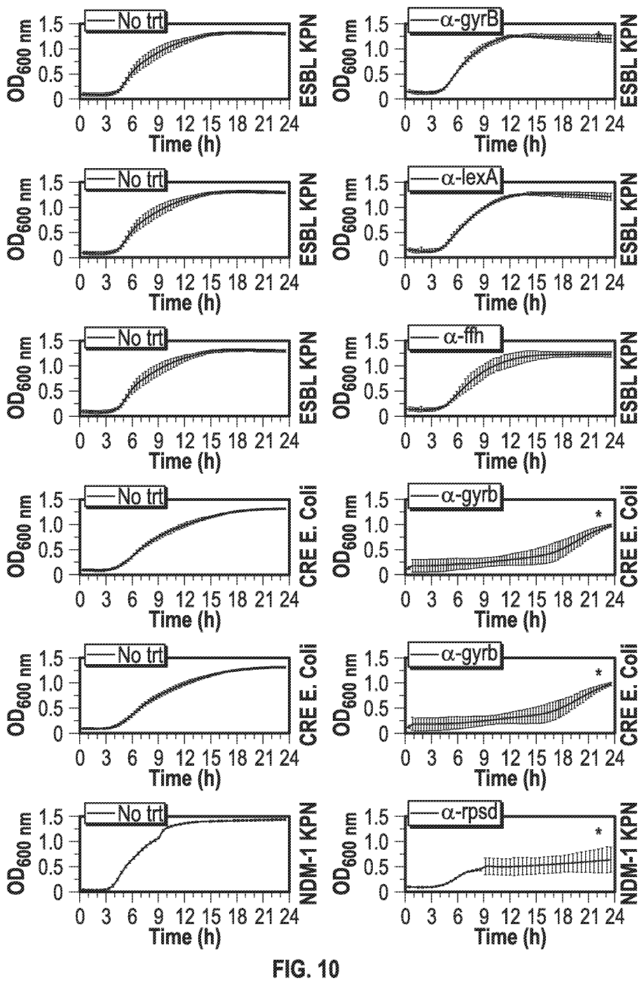

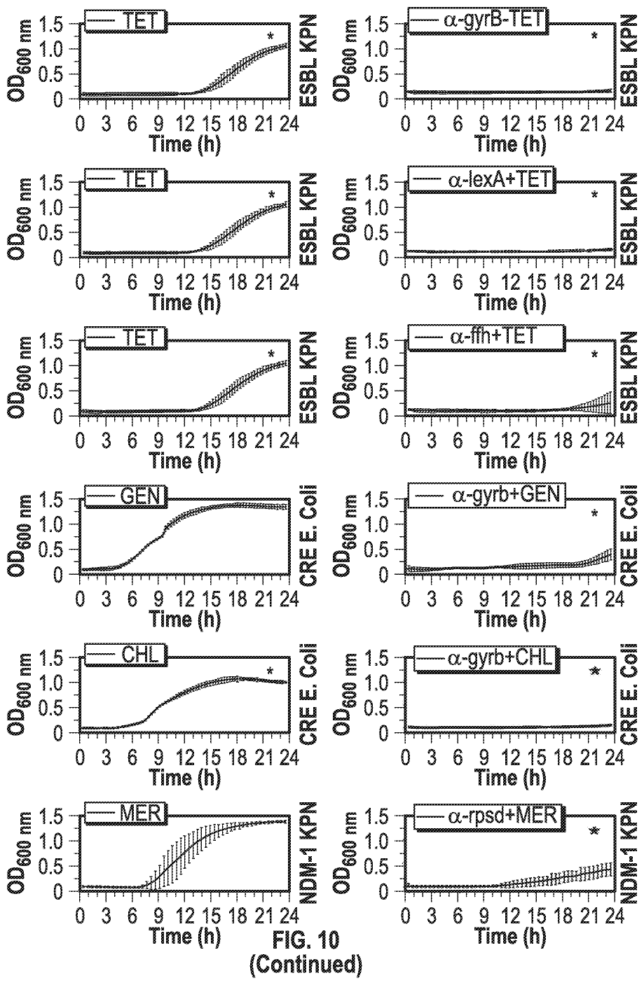

Given the highly resistant phenotype of our clinical isolates and the need for a variety of antimicrobial treatment options, the present inventors next tested the ability of our PNAs to work as potentiators or adjuvants in combination with small-molecule antibiotics. A fraction of the PNAs that were either not effective or partially effective against the clinical isolates at 10 μM were combined with antibiotic concentrations below the minimum inhibitory concentration (MIC) for the isolate. Using the Bliss-Independence model we evaluated the effect of combination (Equation 1), where an S-value>0 indicates synergy. Treatment of CRE E. coli with α-gyrB, which showed a partial therapeutic effect at 10 μM, in combination with antibiotics chloramphenicol (8 μg/mL, FIG. 3A, FIG. 10) and gentamicin (4 μg/mL, FIG. 3B, FIG. 10) led to significant growth inhibition with combination therapy compared to each monotherapy (p<0.05).

This result is striking given that this isolate possessed specific resistance genes for chloramphenicol (PheCml45) and gentamicin (AadB) (FIG. 2A), and was still resensitized to these antibiotics at their CLSI sensitive breakpoints (Table 4). For α-gyrB combination with chloramphenicol and gentamicin, the S-values were 0.27 and 0.19 respectively indicating strong synergy with both antibiotics in CRE E. coli.

The present inventors also evaluated antibiotic potentiation in ESBL KPN using three different PNAs that showed no or minimal growth inhibition as monotherapy at 10 μM: α-ffh, α-lexA, and α-gyrB. These PNAs showed a strong adjuvant activity using 2 μg/mL tetracycline, which is below the CLSI sensitive breakpoint, even though the clinical isolate was capable of surviving at 32 μg/mL tetracycline (FIG. 3C-E). All combinations had significant growth inhibition compared to each monotherapy and a positive S-value, indicating the synergistic effect of the antisense-PNA molecules when added to tetracycline treatment.

The present inventors next investigated a case where the PNA exhibited partial monotherapy effect, but the clinical isolate (NDM-1 KPN) was resistant to the last resort antibiotic meropenem (FIG. 2A, Table 5). The addition of 10 μM α-rpsD and 8 μg/mL meropenem significantly reduced the growth of NDM-1 KPN compared to meropenem monotherapy treatment (FIG. 3F). These results are relevant since PNA monotherapy was not as effective on the KPN isolates, but the combination with a traditional antibiotic strongly inhibited the growth of the isolates. Such synergy of PNA with antibiotics observed in these data can be explained by the fact that targeting multiple protein networks involved in diverse cellular processes may induce fitness deficits resulting in negative epistasis. The present inventors have recently shown, through various techniques, that by expressing an antibiotic resistance gene while disrupting areas of homeostasis including redox, metabolism, and gene expression it is possible to exploit these fitness burdens and resensitize bacteria to antibiotics. The fitness cost of expressing an antibiotic resistance gene in conjunction with perturbation by PNA produce separate and compounding fitness burdens making the bacteria susceptible to previously ineffective antibiotics. These data demonstrate that PNAs that are not as effective as monotherapies can still be highly effective in potentiating the activity of antibiotics in drug-resistance pathogens, expanding the effective supply of usable antibiotics against MDR pathogens.

### Example 4: Using Bacterial Type III Secretion System (T3SS) to Enhance PNA Transport

Despite the tremendous potential of PNAs as both mono- and co-therapies, one of the main challenges limiting their application is their poor uptake by living cells. Since PNA is a hydrophobic molecule it does not transport easily across cell membranes. A number of cellular delivery approaches have been developed including electroporation, co-transfection with DNA, and conjugation to cell-penetrating peptides (CPPs) or nanoparticles. The inventive technology disclosed herein includes a novel approach which takes advantage of the bacterial Type III secretion system (T3SS) to deliver PNAs. The T3SS is a molecular machine used by many gram-negative bacterial pathogens including Salmonella, Shigella, Yersinia, and Pseudomonas, to inject proteins, known as effectors, directly into eukaryotic host cells initiating engulfment and intracellular uptake of bacteria within host cells.

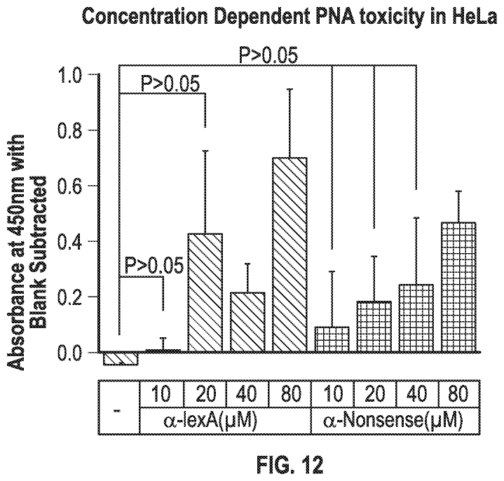

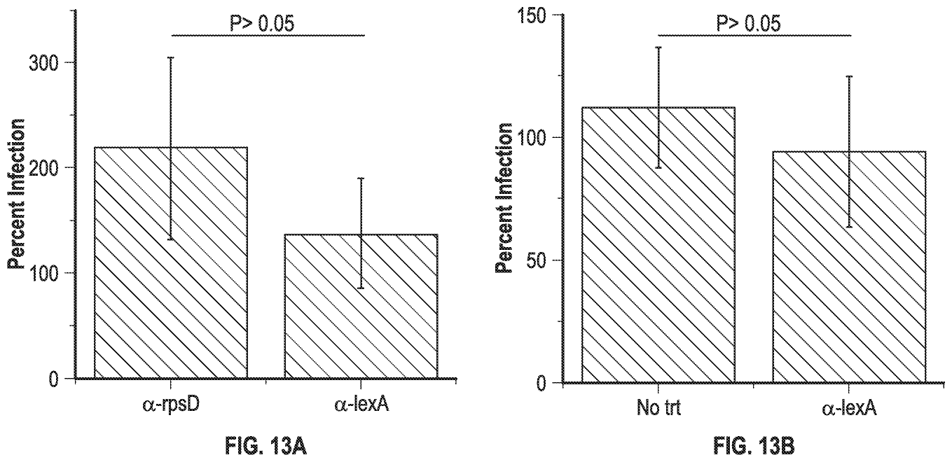

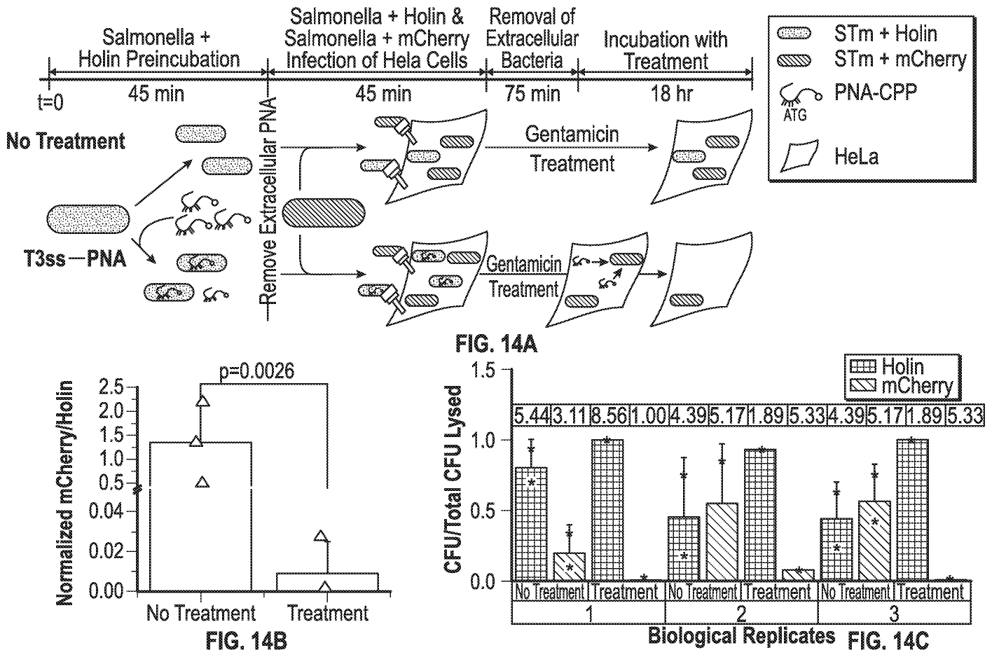

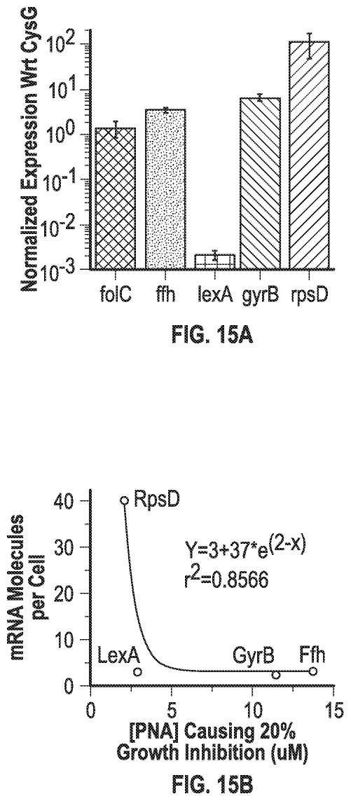

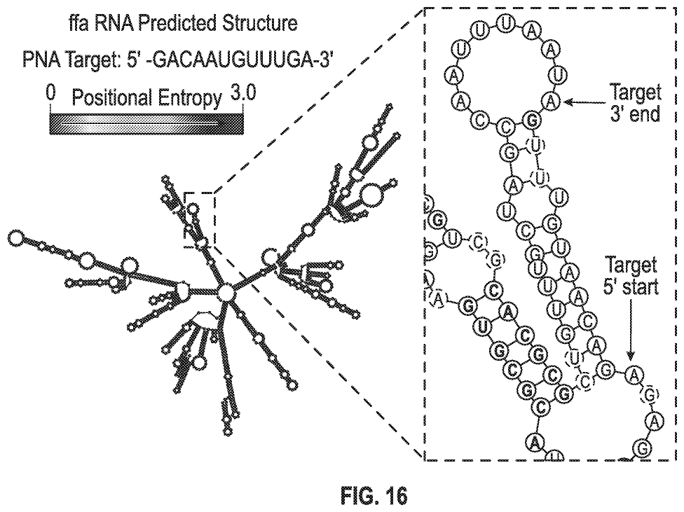

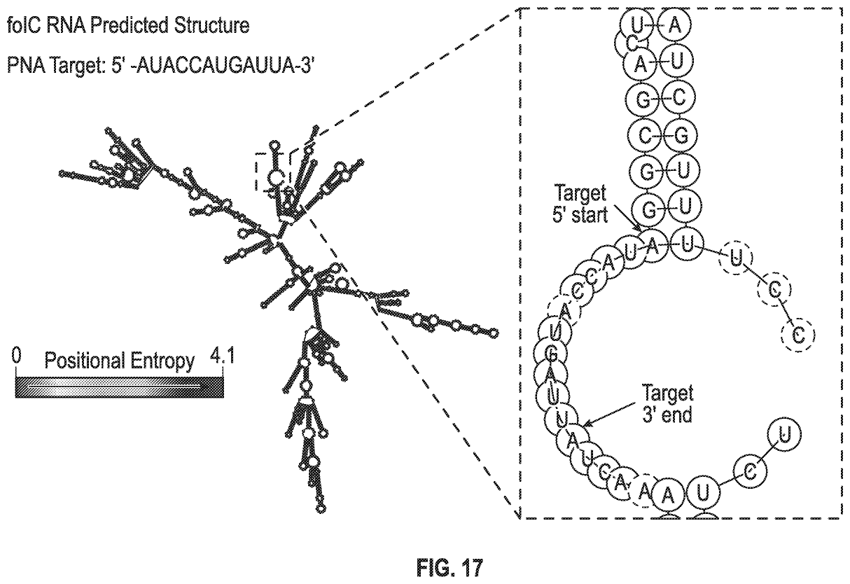

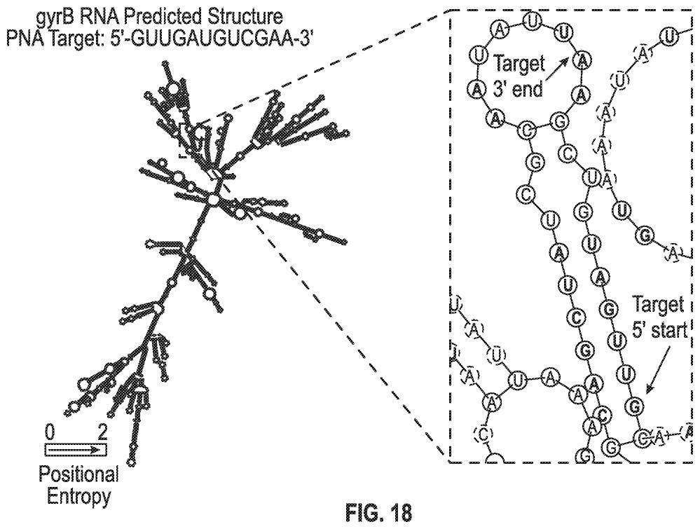

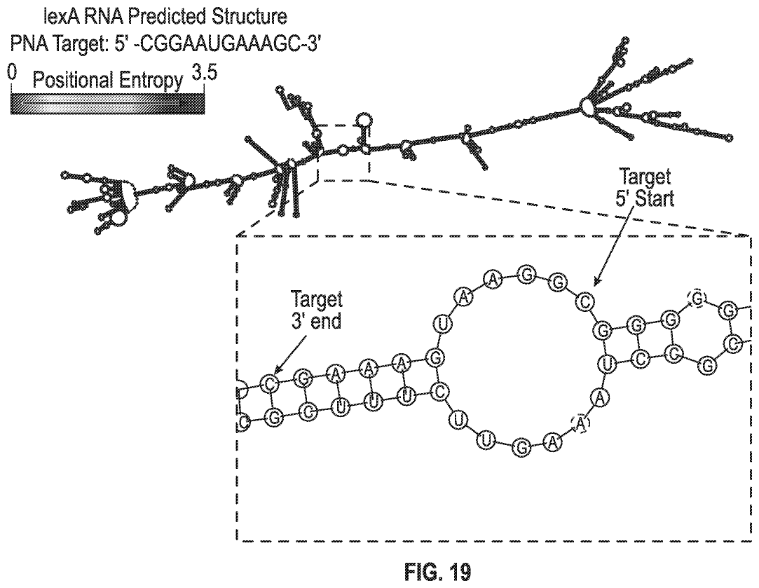

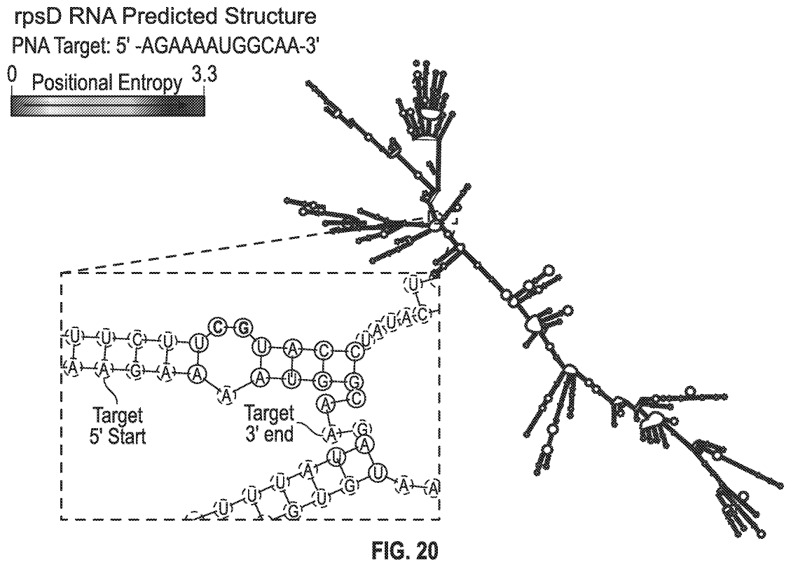

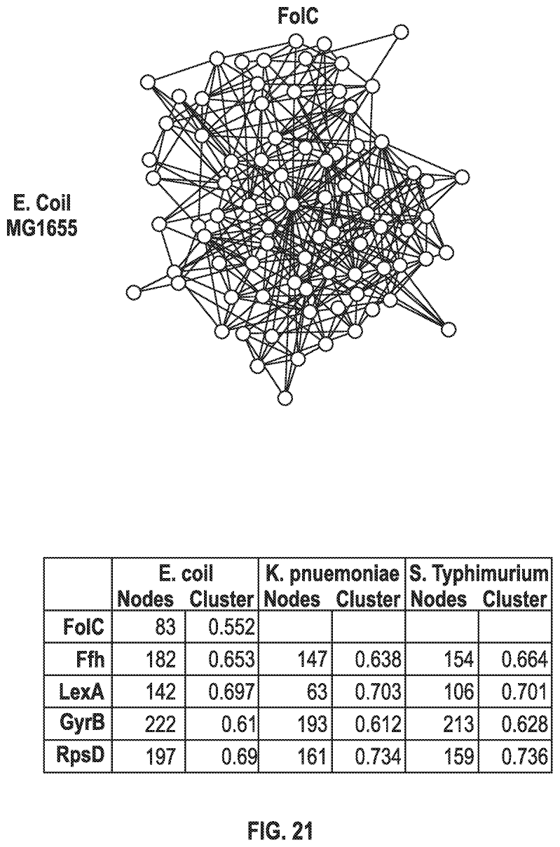

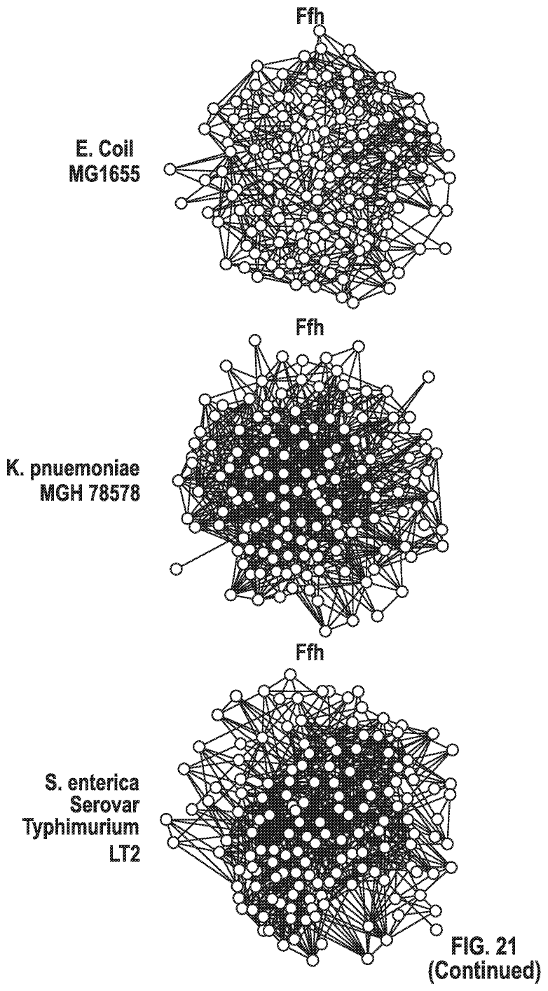

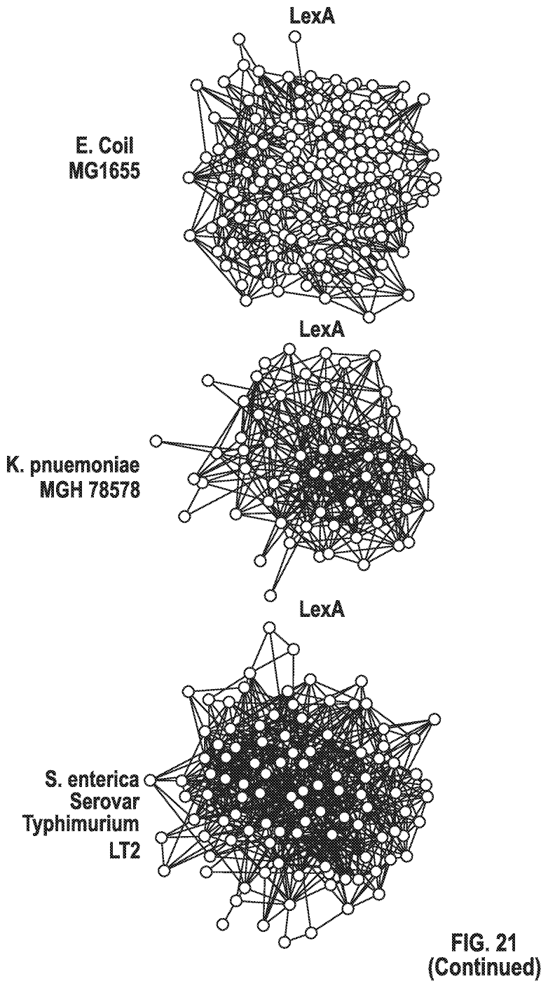

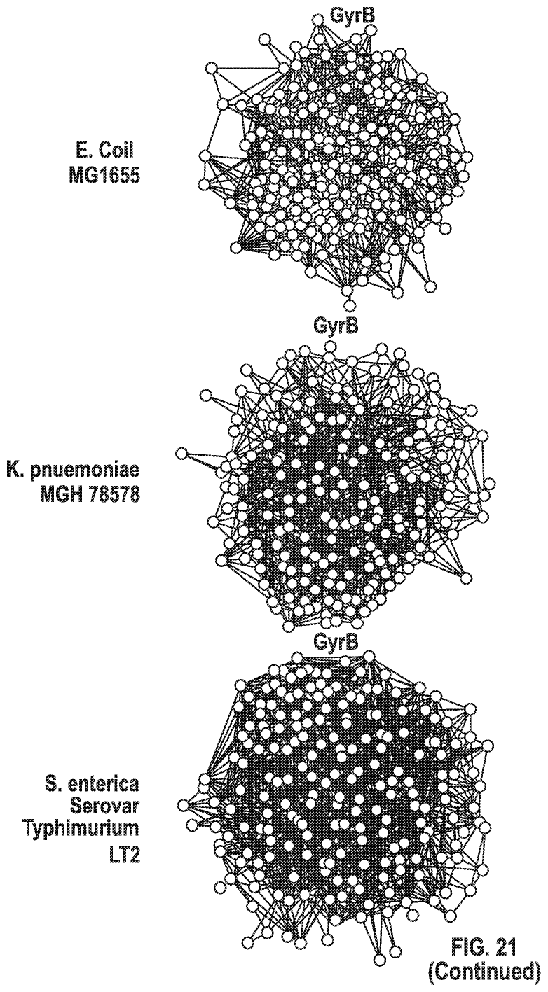

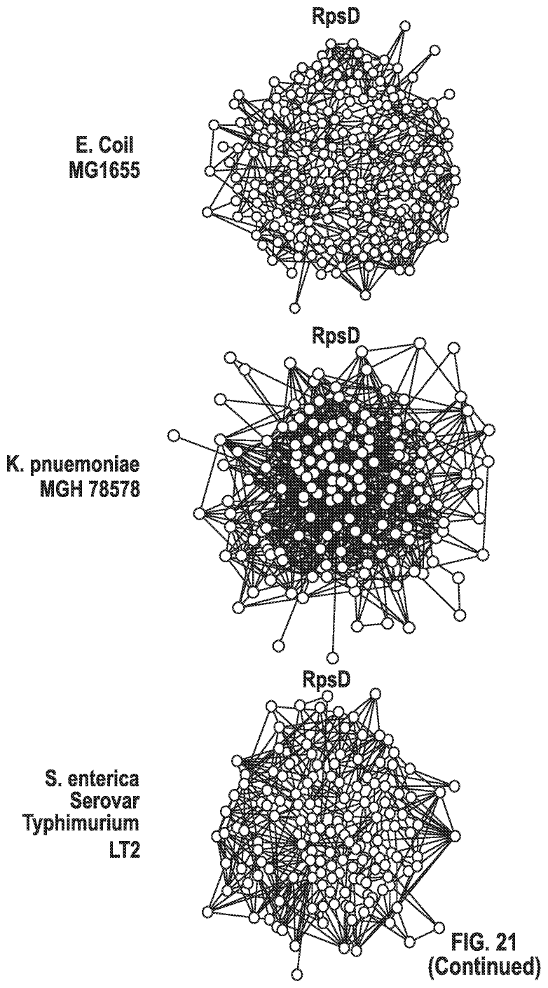

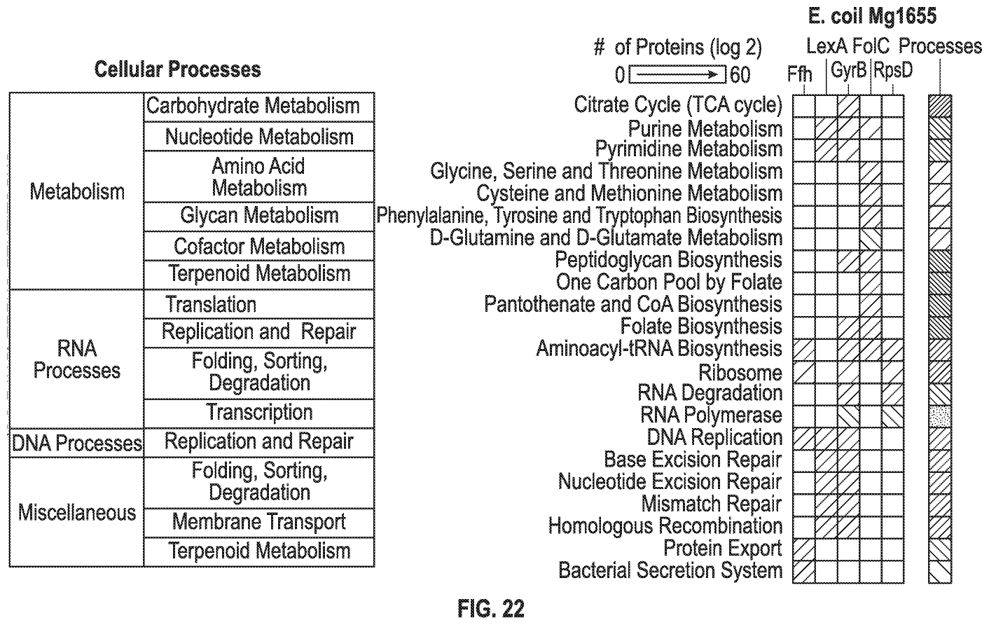

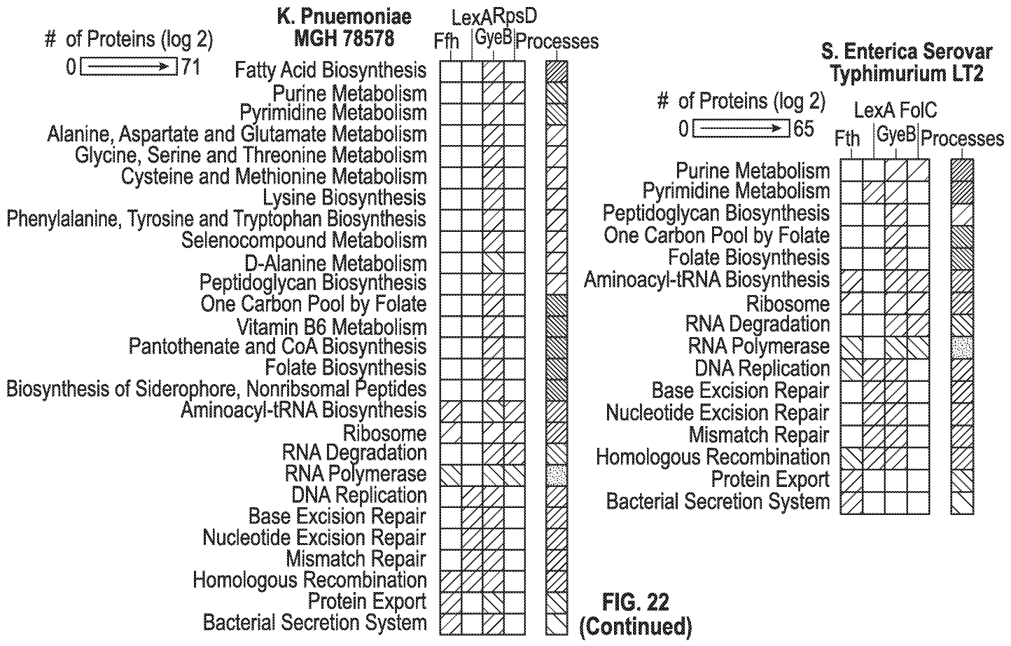

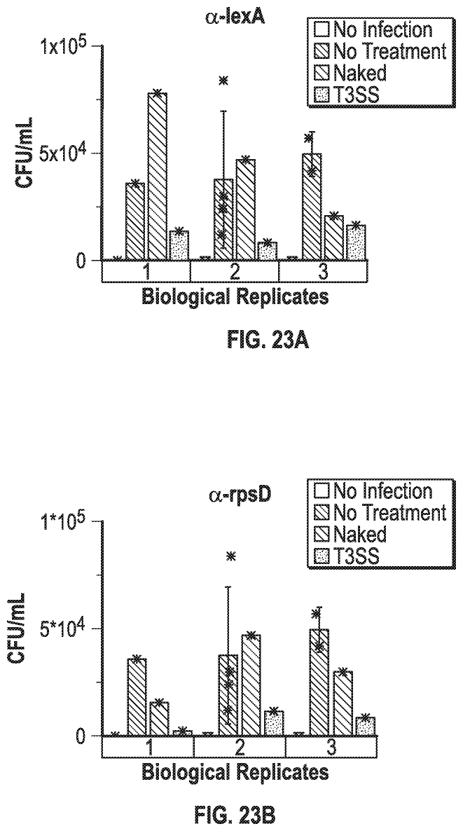

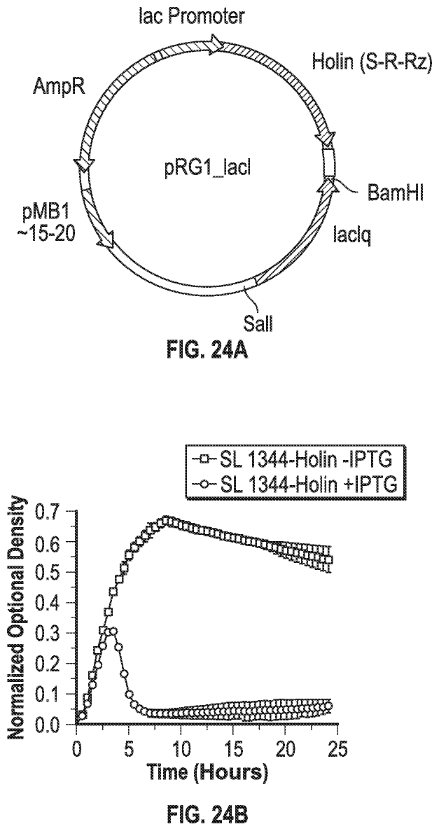

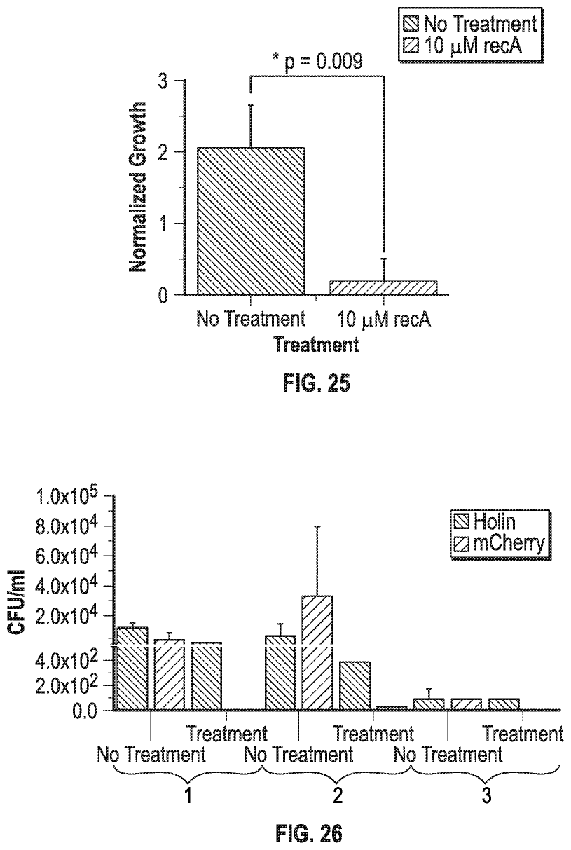

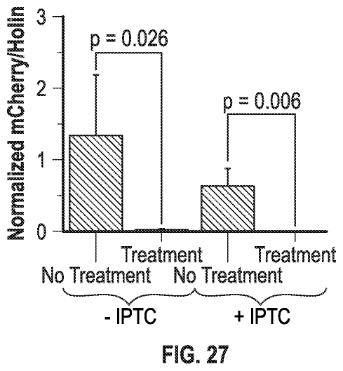

The present inventors utilized the intrinsic T3SS encoded by Salmonella as a delivery vehicle for the designed PNAs (FIG. 4A). The performance of either α-rpsD or α-lexA was investigate given that these were effective against the clinical isolate of Salmonella. These PNA were not found to be lethal to human epithelial cells (HeLa) for a range of concentrations (FIG. 12). To test whether PNAs can eliminate Salmonella enterica serovar typhimurium (strain SL1344 expressing GFP from the chromosome), HeLa cells were infected with Salmonella and treated with PNA molecules using two different methods: one in which PNA was transported via the T3SS (termed as T3SS-PNA treatment), and another in which PNA was added exogenously to the mammalian cells (termed naked PNA treatment) (FIG. 4A).

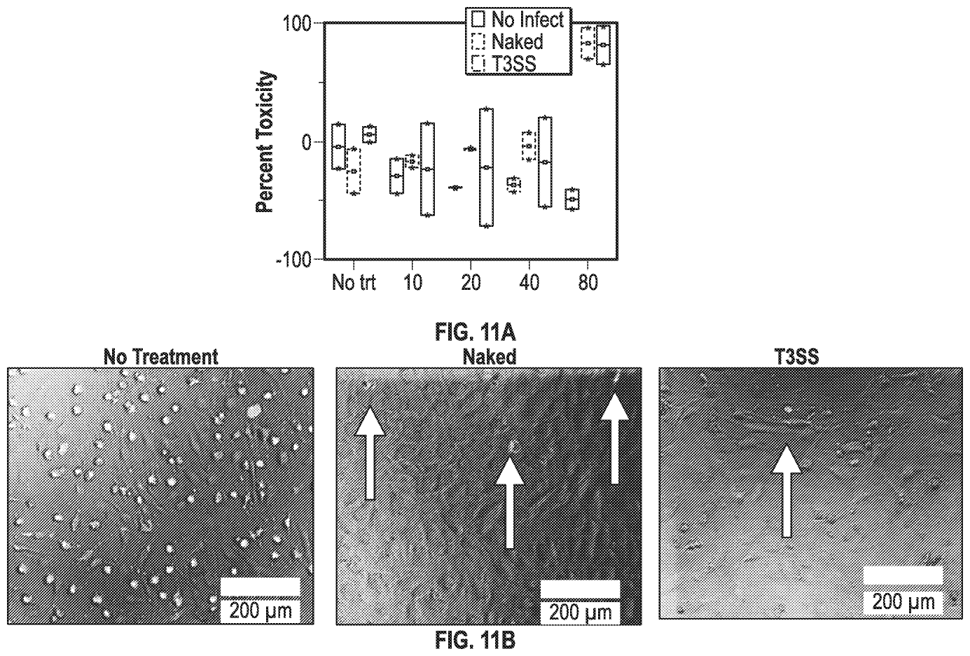

For all cases, HeLa cells were infected at time t=0 with SL1344 for 45 minutes with a multiplicity of infection (MOI) of bacteria to HeLa cells of approximately 10:1. For the T3SS-PNA treatment method HeLa cells were co-incubated with SL1344 and either α-rpsD or α-lexA for 45 minutes, allowing the PNA to enter the Salmonella while simultaneously transporting the PNA-carrying Salmonella into the HeLa cells using the T3SS. This 45 minute co-incubation was not long enough for the PNA to decrease bacterial viability (FIG. 13). Post-infection, the cells were washed and incubated with media containing gentamicin for a period of 75 minutes to remove any extracellular bacteria and/or PNA. For all treatments the media was then replaced with fresh gentamicin containing growth medium, with the naked PNA treatment condition also containing 10 μM PNA, and incubated an additional 18 (FIG. 4A). At the end of 18 hours, cultures were analyzed via imaging or measured by intra-epithelial colony forming units (CFU). Both naked α-lexA and α-rpsD were not effective in clearing an infection compared to no treatment. Contrastingly, α-lexA and α-rpsD delivered via the T3SS both significantly reduced intra-epithelial Salmonella CFU at concentrations of 10 μM by nearly 69±8% and 82±13% respectively (FIG. 4B-C, p<0.05, and FIG. 23). The present inventors obtained similar results in an infection of mouse osteoblast cells (FIG. 11B). These results demonstrate that α-lexA and α-rpsD are capable of diminishing a Salmonella infection and the bacterial T3SS is an effective delivery vehicle of PNAs into a mammalian cells to treat clinically relevant intracellular infections.

### Example 5: Using T3SS and Cell Lysis Switch to Eliminate a Secondary Intracellular Infection

While the T3SS, unaltered, can be used to transport PNA into mammalian cells, infection by Salmonella can lead to the death of the host cell as well as systemic infection; however by combining the T3SS with a lysis switch to release the PNA from the Salmonella, we can transform the T3SS into a therapeutic delivery tool. Here the present inventors implemented a novel method of PNA “delivery and release” to treat a secondary infection, using the T3SS and the naturally programmed cell lysis of the holin-endolysin system. The holin-endolysin system is part of the λ phage lysis cassette which consists of holin, antiholin, and endolysin. Holin functions by accumulating in the inner membrane of the bacteria until a concentration-dependent step causes the proteins to associate and form holes along the inner membrane. The present inventors posited that the T3SS in Salmonella would carry the PNA into infected mammalian cells and the programmed cell lysis will release the intracellular PNA to the target infection for treatment.

To that end, the present inventors built a “delivery strain” of Salmonella by expressing the holin lysis cassette, encoding a holin-antiholin according to SEQ ID NO. 34, under control of an inducible laciQ promoter (referred to as SL1344-Holin, FIG. 24), and a therapeutic “target strain” of Salmonella by expressing mCherry from a plasmid in SL1344 (referred to as SL1344-mCherry). The present inventors followed the same infection protocol as described above with few modifications and treated cells with a different PNA, α-recA (N′-KFFKFFKFFK-AEEA-GTCGATAGCAT-C′), to show the generalizability of the assay. The recA gene is activated by DNA damage to induce the SOS response in bacteria; α-recA PNA was found to be lethal at 10 μM for Salmonella in broth cultures (FIG. 25). As with the previous experiment, the present inventors allowed the Salmonella to uptake the PNA during a 45 minute incubation where the treatment condition (referred to as T3SS-PNA) consisted of SL1344-Holin incubated with 10 μM of α-recA in phosphate buffer saline (PBS) and the no treatment was incubated in only PBS. In parallel, equal cell densities of the target strain SL1344-mCherry was incubated with PBS for 45-minutes. SL1344-Holin (with or without PNA) and SL1344-mCherry cells were washed and mixed in a ratio of 4:1 respectively to achieve a final total MOI of approximately 10:1 for infection of HeLa cells. The infection was carried out for 45 minutes followed by the removal of extracellular bacteria using a gentamicin wash (FIG. 5A), and 18 hours of incubation in fresh growth medium containing gentamicin. Despite fixing the MOI and the initial amounts of SL1344-mCherry or SL1344-Holin used for infection of HeLa cells the extracellular and intracellular bacterial load was found to vary across biological replicates. To account for this variation, the present inventors measured the extracellular and intracellular bacterial load before and after infection respectively and reported the normalized ratio of the latter to the former (FIG. 5B-C, FIG. 26). The SL1344-Holin delivery system significantly reduced the normalized ratio of SL1344-mCherry to SL1344-Holin compared to no treatment with a 99.6±0.72% drop (FIG. 5B, p=0.0026). For two out of three biological replicates, the target strain was completely eliminated across a range of MOI (FIG. 5C, FIG. 26). Furthermore, the present inventors also found that the clearance of the target strain was consistent even at high levels of IPTG induction (FIG. 27). These data provides the first evidence for the use of a combination T3SS and holin-endolysin delivery system to effectively carry and release PNAs to treat a bacterial infection. As note elsewhere, while Salmonella is a pathogenic bacterium in the future T3SS could be expressed heterologously in a probiotic bacterial strain for entry and release of PNA to create a safe and efficient system for PNA transport.

### FIG. 6: Materials and Methods.

Single basepair mismatch off-targets: α-rpsD in S. enterica showed homology with the start site of rtcA (Table 2) when allowing for zero basepair mismatches. The rtcA gene codes for RNA 3′-terminal phosphate cyclase and plays a role in end healing within an RNA repair pathway. When allowing for a 1-bp mismatch on the start codon for all the PNAs, the present inventors found no off-targets for α-lexA and α-gyrB, whereas α-ffh, α-folC, α-rpsD showed 2, 3, 5 off-targets respectively (FIG. 5A, Table 2). A single basepair-mismatch does not necessarily prevent binding to the corresponding RNA sequence but will significantly lower efficiency and increase the minimum inhibitory concentration (MIC). Using an electrophoretic mobility gel shift assay (EMSA) the present inventors find that both α-rpsD (most 1-bp mismatches) and α-lexA (least 1-bp mismatches) show binding specificity to their target (FIG. 5B).

Antibiotic resistant genome screening of clinical isolates: CRE E. coli was found to have five β-lactam resistance genes, two aminoglycoside resistance genes, and one gene each for phenicol, tetracycline, sulfonamide, and trimethoprim resistance. NDM-1 KPN was found to have seven β-lactam resistance genes, three genes each for fluoroquinolone and aminoglycoside resistance and one gene each for tetracycline, sulfonamide, and trimethoprim resistance.

Predicted RNA Folding of PNA targets: The folding of each PNA target theoretical sequence was analyzed using the RNAfold Web. The PNA target regions of ffh (FIG. 16), folC (FIG. 17), and lexA (FIG. 18) all show relatively high local positional entropy, while the rpsD (FIG. 19) and gyrB (FIG. 20) target regions exhibit low entropy. Despite this, both α-gyrB and α-rpsD PNAs were effective in killing both clinical E. coli MDR isolates, as well as wildtype MG1655. This suggests that RNA secondary structure has little influence on PNA efficacy.

Connected cellular pathways and protein network interactions affected by knockdown PNA targets: Based on protein network interactions predictions from the STRING Database (FIG. 21) FolC and LexA tend to interact with few other proteins. Cluster coefficients indicate the former interacts with a diverse set of proteins, while the latter interacts with very specific types of proteins that often also interact with one another. Notably, a cluster of ribosome-related proteins are called in the Ffh and RpsD networks, as can be observed by the standout areas of orange nodes tightly connected to one another. This resulted in relatively high levels of nodes, and generally high levels of cluster coefficient predicted interconnectedness (especially for rpsD). GyrB is particularly interesting in that it both interacts with the most other proteins in each organism and interacts with the most diverse set of proteins with low levels of interconnectedness (excluding FolC). PNA targeting of rpsD is predicted to therefore result in the greatest level of cascading affects throughout dissimilar pathways in the cell and could explain the PNA's relative high success in inducing antibiotic synergy.

Cellular pathway interactions based on PNA target knockdowns (FIG. 22) show that metabolic related pathways are significantly overrepresented in the pool of proteins that FolC interacts with, particularly in relation to amino acid and cofactor synthesis. Proteins related to LexA are heavily involved in a wide range of DNA related processes, as well as nucleotide metabolism. Proteins related to RpsD are very tightly related to RNA processes, and particularly processes related to the ribosome. A few of these processes are also overrepresented in partners of Ffh, which also interacts with miscellaneous membrane and protein export pathways. Most notable is the highly variable pathways that interact with GyrB, covering a wide range of metabolism, RNA, and DNA related processes. This high coverage of pathways related to GyrB could be the reason why the PNA designed to inhibit its expression resulted in the most successful synergy across all organisms and antibiotics tested.

Bioinformatics toolbox for PNA design: Essential genes were identified using the Keio collection entry in the Database of Essential Genes (DEG), which was then used to extract the nucleic acid sequences from the corresponding genome downloaded from the RefSeq database. PNA sequences were extracted using the DEG gene names and a custom Python script as the reverse complements of 12-mer nucleotide sequences centered on the mRNA AUG start codons (STC) for genes of interest. These sequences were screened for off-target alignments within the E. coli genome from which the PNA sequences originated and for homology within the KPN and STm genomes using the Bowtie short-read alignment tool (alignment settings: -v 0-1 12 -a), allowing for no basepair mismatches or gaps within the sequence alignment. The Bedtools “intersect” function was used to identify alignments that overlapped with genome features, and a custom Python script was used to parse this data and calculate the alignments' proximities to gene STCs. Off-target or homology inhibition was defined as a sequence alignment overlapping the STC of a gene that the PNA was not specifically designed to inhibit. Thermodynamic considerations for PNA sequences were screened for using a custom Python script designed to search for potential solubility and self-interference issues. The former was addressed by looking for purine stretches greater than 5 bases, a purine content of greater than 50%, or a guanine-peptide content of greater than 35%. The latter was addressed by looking for self-complementary sequences of greater than 5 bases. PNA target pathways were identified using EcoCyc and Regulon Database.

PNA synthesis: PNAs were ordered from PNA Bio Inc. (Newbury Park, Calif.) conjugated to cell penetrating peptide KFFKFFKFFK (SEQ ID NO. 18). PNA was re-suspended in H₂O with 5% DMSO at 100 μM. Stocks were stored at −20° C. for long-term and at 4° C. for working stocks to minimize freeze/thaw cycles. PNA used for toxicity studies (FIG. 12) was synthesized using solid phase chemistry described in supplemental methods.

Bacterial Cell culture: The clinical isolates were obtained from the lab of Nancy Madinger at the University of Colorado Anschutz campus. Clinical isolates were grown in Cation Adjusted Mueller Hinton broth (CAMHB) (Becton, Dickinson and Company 212322) at 37° C. with 225 rpm shaking or on solid CAMHB with 1.5% agar at 37° C. Clinical isolates were maintained as freezer stocks in 90% CAMHB, 10% glycerol at −80° C. Freezer stocks were streaked out onto solid CAMHB and incubated for 16 h to produce single colonies prior to experiments. For each biological replicate, a single colony was picked from solid media and grown for 16 h in liquid CAMHB prior to experiments. Non-clinical isolates, such as E. coli MG1655 (ATCC700926) and Salmonella entorica serovar typhimurium SL1344, were cultured in liquid 2% lysogeny broth (LB) or on 2% LB with 1.5% agar for solid plates. S. typhimurium cultures were supplemented with 30 μg/mL streptomycin and SL1344 strains containing plasmids pFPV (SL1344-mCherry) or pRG1 modified with a lacIq promoter (SL1344-Holin) were supplemented with 100 μg/mL ampicillin. Freezer stocks were stored in 60% LB broth, 40% glycerol at −80° C. Freezer stocks were streaked out onto solid LB and incubated for 16 h to produce single colonies before experiments. For biological replicates, single colonies were started in liquid LB and grown for 16 hours prior to experiments. E. coli MG1655 PNA growth experiments were carried out in M9 media (1×M9 minimal media salts solution (MP Biomedicals), 2.0 mM MgSO₄, and 0.1 mM CaCl₂ in sterile water) with 0.4% glucose.

Antibiotic resistance screening: Sensitive/resistant breakpoints were taken from the 2016-2017 Clinical & Laboratory Standards Institute report (Table 4). Liquid cultures of the clinical strains were diluted to a 0.5 McFarland standard and added to the respective antibiotic test condition. The antibiotic minimum inhibitory concentration (MIC) for each clinical isolate was determined as the lowest antibiotic concentration which prevented visible cell growth for 24 h. Strains were: “sensitive” if the MIC was equal to or below the sensitive-breakpoint concentration, “resistant” if the MIC was greater than or equal to the resistant-breakpoint concentration, and “intermediate” if the MIC was in-between.

Genome sequencing library prep and data analysis: Liquid cultures were inoculated from individual colonies off of solid cation-adjusted Mueller-Hinton broth (CAMHB) for each clinical isolate. Cultures were grown for 16 h as described above and then 1 mL of culture was used to isolate DNA using the Wizard DNA Purification Kit (Promega). A Nanodrop 2000 (Thermo Scientific) was used to measure DNA concentration and purity. For library preparation, >2 μg of DNA was submitted in 50-100 μL samples. The libraries were prepared for sequencing using Nextera XT DNA Library Preparation Kit (Illumina), and the sequencing was run with a 2×250 bp MiSeq run (Illumina).

Sequencing reads were first trimmed using TRIMMOMATIC v0.32 for length and quality with a sliding window. For further analysis, the trimmed files, of only paired sequences, were transferred to Illumina BaseSpace (http://basespace.illumina.com). We assessed the sequencing quality using FASTQC v1.0.0 and performed de novo genome assembly with SPAdes Genome Assembler v3.6.0. The assembly was further corrected and improved using Rescaf v 1.0.1, and then we performed annotation using PROKKA v1.0.0. Antibiotic resistance genes were identified and characterized using SEAR and ARG-ANNOT pipelines. Integrated Genomics Viewer was used for data visualization.

Homology analysis for PNA in clinical isolates: Clinical isolate genome assemblies with annotation were loaded as contigs into UGENE v1.26.0. In each clinical isolate genome, we searched for each of the five 12-mer target sequences that were selected for the gene-targets. If a target sequence was found to match the start site of the same targeted gene in the clinical isolate we recorded the target as having homology in that genome.

PNA growth assay: Biological replicates were diluted 1:10,000 into treatment condition in 384-well plates and measured for 24 h in a Tecan GENios at 562 nm with a bandwidth of 35 nm. Media absorbance blanks were subtracted from data prior to analysis. Normalized optical density (OD) data is shown normalized to the time point where the “no treatment” growth curves reached saturation phase which varied across biological replicates.

Potentiation of antibiotics with PNAs: Combination growth curve experiments were performed following the same procedure as the PNA growth assay mentioned above, except for the addition of antibiotics tetracycline (2 μg/mL), gentamicin (4 μg/mL), chloramphenicol (8 μg/mL), and meropenem (8 μg/mL) where appropriate. Combinatorial effects were evaluated using the Bliss Independence model where the S parameter defines a deviation from no interaction as is defined as:

$$S = ( \frac{{OD}_{A B}}{{OD}_{0}} ) ( \frac{{OD}_{PNA}}{{OD}_{0}} ) - ( \frac{{OD}_{AB , PNA}}{{OD}_{0}} ) \qquad ( Equation 1 )$$

Where OD_{AB} is the OD at saturation time in only antibiotic, OD₀ is the OD at saturation in no treatment, OD_{PNA} is the OD at saturation in only antisense-PNA at 10 μM, and OD_{AB}, P_{NA} is the OD at saturation in a combination of antibiotic and antisense-PNA. Saturation time was determined as the time when the no treatment control reached its saturation growth phase. S>0 is a deviation towards synergy and S<0 is a deviation towards antagonism.

HeLa Cell Culture: HeLa cells were stored as freezer stocks in 10% dimethyl sulfoxide (DMSO, Sigma) plus fully media: Dulbecco's Modified Eagle Medium (DMEM, Fisher Scientific), 10% Fetal Bovine Serum (FBS, Advanced, Atlanta Biologics), and 50 units/mL Penicillin-Streptomycin (P/S; Fisher Scientific). For biological replicates a single freezer stock, passage two, was split into three different culture flasks and each flask continuously passaged as individual biological replicates in full media. Cells were cultured at 37° C., 5% CO₂, and controlled humidity. Cells were passaged at 80% confluency with 0.25% trypsin (HyClone). HeLa cells were seeded in 96 well tissue culture treated plates (Fisher Scientific) at 45,000 cells/mL respectively in 100 μL per well 24 hours prior to infection.

Salmonella infection of HeLa cells and PNA treatment: For a single infection (FIG. 4), SL1344 cultures were inoculated from a single colony on solid media in to 1 mL of LB media and grown for 16 hours at 37° C. with shaking. Cultures were diluted 1:10 and regrown in LB prior to infection. Bacteria were washed thrice with PBS, optical density at 600 nm measured, and diluted to a multiplicity of infection (MOI) of 10 in Dulbecco PBS (DPBS). MOI is calculated based on an established optical density and colony forming unit (CFU) calibration, and the number of mammalian cells per well at the time of infection approximated based on their doubling time. Mammalian cells were washed thrice with Dulbecco's Phosphate-Buffered Saline (DPBS, Fisher Scientific) prior to infection for 45 minutes 37° C. with controlled humidity. Media containing bacteria for infection was replaced with 40 μg/mL gentamicin in DPBS to remove any extracellular bacteria and incubated for 75 minutes before replacement with cell culture media and respective treatment.

At 18 h post-infection, wells for CFU analysis were washed thrice with 300 μL DPBS and lysed with 30 μL of 0.1% Triton X-100 for 15 minutes at room temperature. After 15 min, 270 μL of PBS was added to each well then serially diluted and plated on sold LB media supplemented with 40 μg/mL streptomycin. Plates were incubated for 16 hours at 37° C. and CFU per milliliter determined. Wells for staining and imaging were fixed with 4% methanol-free paraformaldehyde at room temperature for 20 minutes. Staining for nuclei was performed using 100 μL per well of 2.5 μg/mL DAPI (Santa Cruz Biotechnology) for 5 minutes at room temperature then washed thrice with DPBS before staining with 100 μL of 0.165 μM Alexa Fluor 647 Phalloidin (Thermo Fisher) supplemented with 0.25% Triton X-100 for 20 minutes at room temperature. Cells were rinsed with DPBS and stored in 65% glycerol at 4° C. Images were acquired using an EVOS FL microscope and analyzed using ImageJ.

Double infection of HeLa cells and PNA treatment: For the double infection of HeLa cells with SL1344-mCherry and SL1344-Holin (FIG. 5A-C), single colonies were picked on solid media and grown overnight in 1 mL of LB broth supplemented with 30 μg/mL Streptomycin and 100 μg/mL Ampicillin. Overnight cultures were diluted 1:10 and regrown for 3 hours. After regrowth the cultures were washed thrice with PBS and diluted to a concentration of to 9×10⁶ CFU/mL (MOI of 10). The SL1344-Holin infection stock was split into no treatment and treatment; 10 μM of recA was added to the treatment condition and an equal volume of PBS was added to the no treatment. Both conditions were incubated for 45 minutes at 37° C., mimicking the conditions used in the single infection experiment for the PNA to enter the Salmonella. After incubation all infection stocks (SL1344-mCherry, SL1344-Holin no treatment, and SL1344-Holin with treatment) were washed thrice with DPBS to remove any extracellular PNA. Each SL1344-Holin and SL1344-mCherry stock was combined at a 4:1 ratio to create the treatment and no treatment infection solutions and 50 μL was to infect the HeLa. To ensure that the incubation ratio remained the same and PNA waste was minimized the DPBS washes were done with volumes under 1 mL resulting in variability of the actual MOI infected with of each infection stock. To account for this variability each infection stock was serially diluted, plated, grown overnight, and CFUs counted to determine the exact amount of bacteria that was used to infect for each condition. This extracellular CFU causing infection was measured for both SL1344-Holin and SL1344-mCherry using florescence and is referred to as CFU_{Holin}^{Infected With} and CFU_{SL1344-mCherry}^{Infected With} respectively. For CFU counting the separate bacterial populations were separated by counting the SL1344-mCherry as those colonies that fluoresced when excited by light at 587 nm in a light box and viewed through a 610 nm emission filter, and the SL1344-Holin colonies as colonies showing lack of fluorescence. At 18 h post-infection wells for CFU analysis were washed thrice with 300 μL PBS and lysed with 30 μL of 0.1% Triton X-100 for 15 minutes at room temperature. After 15 min, 270 μL of PBS was added to each well then serially diluted and plated on sold LB media supplemented with 40 μg/mL streptomycin. Plates were incubated for 16 hours at 37° C. and CFU per milliliter determined. The intracellular CFU post lysis was measured for both SL1344-Holin and SL1344-mCherry using florescence and is referred to as CFU_{Holin}^{Lysed} and CFU_{SL1344-mCherry}^{Lysed} respectively. To account for variability of MOI we normalized the CFU of SL1344-Holin and SL1344-mCherry cells using the ratio (CFU_{SL1344-mCherry}^{Lysed}/CFU_{SL1344-mCherry}^{Infected With}) and (CFU_{Holin}^{Lysed}/CFU_{Holin}^{Infected With}) respectively.

Effect of PNA after 45 min of incubation during HeLa infection: SL1344 were cultured as described above and split 1:10 and regrown for 4 hours. Samples were rinsed with PBS and diluted to a concentration equivalent to an MOI of 30 for a 24 hour grown culture of HeLa cells in 100 μL. PNA (100 mM) was added at 5 μL to 45 μL of bacteria and incubated for 45 minutes at culturing conditions. Samples (10 μL) were taken at t=0 (before PNA addition) and t=45 min (after PNA treatment), and serially diluted, plated, and grown for 16 hours at 37° C. and CFU counted.

Error and Significance Analysis: Error bars represent one standard deviation from the mean of biological replicates. In all cases, significance designated with an asterisk (*) is defined as p<0.05 using a Student's t test.

PNA Synthesis: Synthesis of the PNA molecules was carried out in solid-phase on Fmoc-MBHA resin (0.22 mmol/g loading) at a 0.02 millimolar scale. Fmoc deprotection steps were performed using piperidine and coupling was performed using HATU as an activator. Post-coupling acetylation was carried out with a 5%/6% v/v solution of acetic anhydride and 2,6-lutidine, respectively, in DMF. Cleavage was performed for 2 hours using a 88%/2%/5%/5% v/v solution of TFA, TIPS, phenol, and water, respectively. The PNA molecule was precipitated in ethyl ether, centrifuged, and the purified using high pressure liquid chromatography.

Gel Shift Mobility Assay: Synthetic DNA oligonucleotides that are 57-60 nucleotides in length were incubated with their respective PNA binding site at 37° C. overnight. The concentration of the DNA fragments was held at 500 nM, while the PNA was always in excess at 1 μM. The reactions were performed in 1×TE with 20 mM KCl (pH 7.0). The formation of PNA-ssDNA complexes was observed on a 20% polyacrylamide nondenaturing gel using 1×TBE running buffer. 1×SYBR-Gold® was used to stain and visualize the DNA using about 50 mL to coat the gel in low-light conditions for 20 minutes. The gel was directly put on a UV sample tray and imaged using the Gel Doc™ EZ Imaging system from Bio-Rad.

Plasmid and strain construction: The SL1344-Holin strain contains a modified pRG1 plasmid that is IPTG inducible. The original pRG1 plasmid expresses the SRRz genes of the lysis cassette.⁸ The lacIq gene was inserted in between the SalI and BamHI cut sites. The lacIq gene was extracted from the E. coli strain DH5αz1 by colony PCR with Phusion High-Fidelity DNA Polymerase (New England Biolabs) and with the following primers: forward primer (5′-AAAGGATCCCATCACTGCCCGCTTTCCAGTCG-3′) and reverse primer (5′-AAAGTCGACCCGACACCATCGAATGGTGCAAAACCTTTCG-3′). The PCR products were subsequently gel-purified (Zymoclean Gel DNA Recovery Kit, Zymo Research Corporation), digested sequentially with BamHI and SaII (FastDigest Enzymes, Thermo Scientific) as per provided protocols, and PCR-purified (GeneJET PCR Purification Kit, Thermo Scientific) between and after digestion. The pRG1 backbone was also digested with BamHI and SaII and gel purified, and T4 DNA Ligase (Thermo Scientific) was used to ligate the pRG1 backbone and the extracted lacIq gene. Ligations were transformed into electrocompetent SL1344 cells and plasmid minipreps were performed using Zyppy Plasmid Miniprep Kit (Zymo Research). Confirmation was done by measuring the optical density with and without 1 mM IPTG (FIG. 27). The SL1344-mCherry bacterial strain contains the pFPV-mCherry plasmid (Addgene #20956) which was transformed into electrocompetent SL1344 cells and confirmed by fluorescence when excited with light at 587 nm in a light box and viewed through a 610 nm emissions filter.

Optical Density Growth Measurements of SL1344-Holin: Single colonies of each bacterial strain were picked and grown in LB supplemented with 30 μg/mL Streptomycin and 100 μg/mL Ampicillin. Overnight cultures were diluted 1:1,000 into 100 μL of LB supplemented with 30 μg/mL Streptomycin and 100 μg/mL Ampicillin and 1 mM IPTG where appropriate in a 96-well plate. Cultures were grown at 37° C., with shaking, for 24 hours and OD₆₀₀n measured every 30 minutes using a GENios plate reader (Tecan Group Ltd.) operating under Magellan software (version 7.2).

String Database Analysis: Gene names for each of the PNA-targeted RNA sequences were entered into the STRING database and searched in the organisms of Escherichia coli K12 MG1655, Klebsiella pneumoniae subspecies pneumoniae MGH 78578, and Salmonella enterica subspecies enterica serovar typhimurium strain LT2. The meaning of network edges was set to confidence, and all active interaction sources were used. The minimum interaction score was left at 0.400, while the max number of interactions was set to 250 for the 1^{st} shell. Counts of nodes and clusters were taken from the analysis tab. Counts of significantly enriched KEGG⁹ pathway IDs were extracted from the analysis tab.

Quantitative real-time polymerase chain reaction: Individual colonies of E. coli MG1655 were grown 16 h in liquid and subsequently diluted 1:10,000 into liquid media. Cells were collected when they reached exponential phase; determined as OD 0.4-0.5. 200 μL of culture was added to Bacteria RNAprotect (Qiagen) and pelleted following the manufacturer's instructions. Samples were flash frozen in an ethanol-dry ice bath and stored at −80° C. RNaseZap (Life Technologies) was used to protect extracted RNA from RNases. GeneJET RNA purification kit (Thermo Scientific) was used to extract RNA from frozen cell pellets followed by treatment with Turbo DNA-free kit (Ambion). 100 ng of cDNA was synthesized using Maxima Universal First Stand cDNA synthesis kit (Thermo Scientific). Primers for qPCR, listed in Table 7, were purchased from Integrated DNA Technologies. 1.5 ng of cDNA was used for 10 μL qPCR reaction with Maxima SYBR Green qPCR master mix with ROX normalization (Thermo Scientific) using QuantStudio 6 flex system (Thermo Scientific). Transcript levels were analyzed following the ΔCq method concerning moderately expressed housekeeping gene cysG.

Osteoblast Precursor Cell Culture: MC3T3-E1 osteoblast precursor cells were stored in liquid nitrogen in growth media supplemented with %10 dimethyl sulfoxide (DMSO, Sigma). Osteoblasts were recovered from freezer stocks and cultured in growth media consisting of α-Minimum Essential Media (α-MEM, Gibco) supplemented with 10% Fetal Bovine Serum (FBS, Advanced, Atlanta Biologics), and 50 units/mL Penicillin-Streptomycin (P/S, Fisher Scientific) at 37° C., 5% CO₂, and controlled humidity. For biological replicates a freezer stock, at passage 9, was split three ways and each replicate continuously passaged as individual biological replicates at 80% confluency using 0.25% trypsin (HyClone). For toxicity experiments osteoblasts, between passages 10 and 15, were seeded on to 96-well tissue culture treated plates (Fisher Scientific) at 100,000 cells/mL in 100 μL 24 hours before infection or treatment.

Osteoblast Precursor Infection and Toxicity Measurement: Osteoblast cells were infected by replacing the growth media with Dulbecco's phosphate-buffered saline containing Salmonella at a concentration equivalent to a multiplicity of infection of 30 and incubated at growth conditions for 45 minutes. After infection media was replaced with growth media supplemented with 30 μg/mL gentamicin instead of Penicillin-Streptomycin, incubated for 75 minutes, and replaced with fresh gentamicin containing media and treatment conditions (150 μL per well). After 18 hours of treatment 90 μL of supernatant was used to determine lactate dehydrogenase (LDH) release as a measure of cytotoxicity using the CytoSelect™ LDH Cytotoxicity Assay Kit. Percent toxicity is defined and measured as:

$$Percent Toxicity = \frac{( {Abs}_{450 nm}^{Treatment} - {Abs}_{450 nm}^{NegativeControl} )}{( {Abs}_{450 nm}^{PositiveControl} - {Abs}_{450 nm}^{NegativeContorl} )} \qquad ( Equation S1 )$$

### TABLES

TABLE 1
Homology between mRNA and PNA of bacterial reference
sequences. Antisense-PNA molecules ordered from PNA
Bio Inc. The target sequence is bolded and underlined
and the start codon is italicized.

| | Pathogens | | |
|---|---|---|---|
| | with | Sequence of the | |
| PNA | homology | targeted mRNA (5′ to 3′) | SEQ ID NO. |
| folC | E. coli | AGCGG [symbol] TCAAACG | SEQ ID NO. 1 |
| rpsD | E. coli | TGGAGAA [symbol] GATATTT | SEQ ID NO. 2 |
| | K. pneumoniae | TGGAGAA [symbol] GATATTT | SEQ ID NO. 3 |
| | S. enterica | TGGAGAA [symbol] GATATTT | SEQ ID NO. 4 |
| | S. enterica, | AAAGGAT [symbol] GGATCAT | SEQ ID NO. 5 |
| | rtcA | | |
| ffh | E. coli | GGCGAGA [symbol] TAATTTA | SEQ ID NO. 6 |
| | K. pneumoniae | GGCGAGA [symbol] TAATTTA | SEQ ID NO. 7 |
| | S. enterica | GGCGAGA [symbol] TAATTTA | SEQ ID NO. 8 |
| lexA | E. coli | CAGGGGG [symbol] GTTAACG | SEQ ID NO. 9 |
| | K. pneumoniae | CAGGGGG [symbol] GTTAACG | SEQ ID NO. 10 |
| | S. enterica | CAGGGGG [symbol] GTTAACG | SEQ ID NO. 11 |
| gyrB | E. coli | GAGAAAC [symbol] TTCTTAT | SEQ ID NO. 12 |
| | K. pneumoniae | GAGAAAC [symbol] TTCTTAT | SEQ ID NO. 13 |
| | S. enterica | GAGAAAC [symbol] TTCTTAT | SEQ ID NO. 14 |

TABLE 2
Predicted 0-bp mismatch off-targets of antisense-PNA molecules in each reference bacterial
genome and clinical isolate. The label “STC” denotes gene off-targets for which the
antisense-PNA aligns to the start codon, and which will therefore be much more
likely to experience translational inhibition.
Bacterial Reference Genome Results

| | | | Salmonella |
|---|---|---|---|
| | | | enterica serovar |
| | | | Typhimurium |
| PNA | E. coli MG1655 | K. pneumoniae MGH 78578 | SL1344 |
| α-folC | None | KPN_04193 (putative 6-phosphofructokinase) | None |
| | | uxaC | |
| α-rpsD | cueO | Non-protein coding region | wcaM |
| | narI | | rtcA (STC) |
| α-ffh | None | None | None |
| α-lexA | None | None | None |
| α-gyrB | Last 5 nt of psiE | None | None |

Clinical Isolates Results

| | | MDR E. | | | |
|---|---|---|---|---|---|
| | CRE E. coli | coli | ESBL KPN | NDM-1 KPN | STm |
| α-folC | Prokka 00542 | None | pfka1 | pfkA1 | None |
| | Prokka 03005 | | | | |
| α-rpsD | cueO | cueO | frlD | Non-protein | Prokka03791 |
| | | | Non-protein | coding region | Prokka 03488 |
| | | | coding region | | rtcA (STC) |
| α-ffh | uhpC | uhpC | yhes1 | yheS2 | None |
| α-lexA | None | None | None | None | None |
| α-gyrB | Non-protein | None | Non-protein | None | None |
| | coding region | | coding region | | |
| | Last 5 bp of yhbX | | | | |

TABLE 3
DNA oligonucleotides containing PNA-target gene
sequence that were utilized for DNA-PNA complex
formation in EMSA. Synthetic DNA oligonucleotides
purchased from IDT that are 60nt in length,
contained the PNA-target gene sequence bolded and
underlined. The nonsense oligonucleotide was a
randomized 60nt sequence that had no complementation
for any PNA binding, that served as a non-binding
control.

| Oligo/ | | | |
|---|---|---|---|
| Primer | Target | | |
| Purpose | gene | Oligo/Primer Sequence (5′ to 3′) | SEQ ID NO. |
| Antisense | rpsD | ATT TAG GTG ACA CTA TAG AAG TGG | SEQ ID NO. 20 |
| oligomer | | AGA AAG [symbol] AGA TAT TTG | |
| for | | GGT CCT AAG CTC | |
| α-rpsD | | | |
| Antisense | lexA | ATT TAG GTG ACA CTA TAG AAG CAG | SEQ ID NO. 21 |
| oligomer | | GGG GCG GA [symbol] AAG CGT TAA CGG | |
| for | | CCA GGC AAC AAG | |
| α-lexA | | | |
| Nonsense | N/A | GAA TTC GAA TTC GGT CAG TGC GTC | SEQ ID NO. 22 |
| oligomer | | CTG CTG ATG TGC TCA GTA TCT CTA | |
| | | TCA CTG ATA GGG | |

TABLE 4
CLSI sensitive/resistant breakpoints. CLSI breakpoints (μg/mL)
for 2016-2017 were used to determine antibiotic resistance
of clinical isolates.

| Antibiotic | Sensitive | Intermediate | Resistant |
|---|---|---|---|
| Ampicillin (AMP) | 8 | 16 | 32 |
| Ceftriaxone (FRX) | 1 | 2 | 4 |
| Meropenem (MER) | 1 | 2 | 4 |
| Gentamicin (GEN) | 4 | 8 | 16 |
| Kanamycin (KAN) | 16 | 32 | 64 |
| Tetracycline (TET) | 4 | 8 | 16 |
| Ciprofloxacin (CIP) | 1 | 2 | 4 |
| (E. coli and K. pneumoniae) | | | |
| Ciprofloxacin | 0.06 | 0.125 | 1 |
| (Salmonella enterica) | | | |
| Nalidixic Acid (NXA) | 6 | N/A | 32 |
| Chloramphenicol (CHL) | 8 | 16 | 32 |

TABLE 5
Unique antibiotic resistance genes identified in clinical isolates.
Italicized label represents antibiotic class the gene confers
resistance to where: Bla is for β-lactam resistance, Flq is
for fluoroquinolone resistance, AGly is for amino-
glycoside resistance, Phe is for phenicol resistance,
Tet is for tetracycline resistance, Sul is for
sulfonamide resistance, and Tmt if for
trimethoprim resistance. Non-italicized
portion is the unique gene identified by
ARG-ANNOT.

| CRE E. coli | MDR E. coli | ESBL KPN | NDM-1 KPN | STm |
|---|---|---|---|---|
| Bla AmpC1 | Bla AmpC2 | Bla SHV-11 | Bla TEM-217 | AGly |
| Bla AmpH | Bla PBP | Bla AmpH | Bla NDM-1 | Aac6-Iaa |
| Bla AmpC2 | Bla ampH | Bla Oxa-9 | Bla CTX-M | Bla PBP |
| Bla CMY-94 | Bla TEM-219 | Bla TEM-171 | Bla SHV-73 | |
| Bla PBP | Bla TEM-10 | Bla TEM-220 | Bla PBP | |
| AGly AadB | Bla CTX-M | Bla KPC-3 | Bla AmpH | |
| AGly StrA/B | AGly Sat-2A | Bla PBP | Bla PBP | |
| Phe PheCm145 | Tmt DfrA1 | Flq OqxBgb | Flq QnrB1 | |
| Tet TetB | Phe CatA1 | AGly | Flq Qnr-S1 | |
| Sul SulI | | AadA1-pm | Flq OqxBgb | |
| Tmt Dfr24 | | AGly Aac6-Ib | AGly StrB | |
| | | | AGly RmtF | |
| | | | AGly Ant3 | |
| | | | Tet TetA/R | |
| | | | Sul SulI | |
| | | | Tmt DfrA1 | |

TABLE 6
Genetic and phenotypic antibiotic resistance characterization. Red cells
correspond to full resistance across an antibiotic class. Yellow is partial
resistance to the class, and green is full sensitivity to the class.

| | CRE E. | MDR E. | ESBL | NDM-1 | |
|---|---|---|---|---|---|
| | coli | coli | KPN | KPN | STm |
| | # of | # of | # of | # of | # of |
| Antibiotic | Resistance | Resistance | Resistance | Resistance | Resistance |
| Type | Genes | Genes | Genes | Genes | Genes |
| β-lactams | 5 | 6 | 7 | 7 | 1 |
| Amino- | 2 | 1 | 2 | 3 | 1 |
| glycosides | | | | | |
| Tetracyclines | 1 | 0 | 0 | 1 | 0 |
| Fluorquin- | 0 | 0 | 1 | 3 | 0 |
| olones | | | | | |
| Phenicols | 1 | 1 | 0 | 0 | 0 |

TABLE 7
RT-qPCR primers used for gene expression analysis.
The length of qPCR products are between 160 and 200 nt.

| | Forward Primer | Reverse Primer | Product | |
|---|---|---|---|---|
| Gene | (5′→3′) | (5′→3′) | Length | SEQ ID NO. |
| gyrB | CGGGTCCATAGTGGTTTCCC | GTGAGAAACTGCGTGGCTTG | 191 | SEQ ID NO. 24, 25 |
| folC | GCTCAAGCAGTTGTTCTGCC | TCTCACCGGGCGTATGAAAG | 176 | SEQ ID NO. 26, 27 |
| ffh | TTCCATACGCACCAGCACTT | CGCGCAGGCAGAGAAATTAG | 193 | SEQ ID NO. 28, 29 |
| rpsD | CAGCCAGGTTGGCTTTTCAC | AGAAGCACGTCAGCTGGTTA | 178 | SEQ ID NO. 30, 31 |
| lexA | GTTAACGGCCAGGCAACAAG | TCAATAACGCCTTTGCGTGC | 162 | SEQ ID NO. 32, 33 |

TABLE 8
PNAs Library sequences.

| | | |
|---|---|---|
| ATACCATGATTA | α-folC | SEQ ID NO. 15 |
| GACAATGTTTGA | α-ffh | SEQ ID NO. 16 |
| cGGAATGAAAGC | α-lexA | SEQ ID NO. 17 |
| GTTGATGTCGAA | α-gyrB | SEQ ID NO. 18 |
| AGAAAATGGCAA | α-rpsD | SEQ ID NO. 19 |

### REFERENCES

The following references are incorporated into the specification in their entirety:
1. United States Center for Disease Control. Antibiotic Resistance Threats. (2013).
2. Tacconelli, E. & Magrini, N. Global priority list of antibiotic-resistant bacteria to guide research, discovery, and development of new antibiotics. (2017).
3. Baba, T. et al. Construction of Escherichia coli K-12 in-frame, single-gene knockout mutants: the Keio collection. Mol. Syst. Biol. 2, 1-11 (2006).
4. Courtney, C. & Chatterjee, A. cis-Antisense RNA and Transcriptional Interference: Coupled Layers of Gene Regulation. J. Gene Ther. 2, 1-9 (2014).
5. Dryselius, R., Aswasti, S. K., Rajarao, G. K., Nielsen, P. E. & Good, L. The translation start codon region is sensitive to antisense PNA inhibition in Escherichia coli. Oligonucleotides 13, 427-33 (2003).
6. V. V. Demidov, V. N. Potaman, M. D. Frank-Kamenetskii, M. Egholm, O. Buchardt, S. H. Sonnichsen, P. E. N. Stability of peptide nucleic acids in human serum and cellular extracts. Biochem. Pharmacol. 48, 1310-1313 (1994).
7. Nielsen, P. E., Egholm, M., Berg, R. H. & Buchardt, O. Sequence-selective recognition of DNA by strand displacement with a thymine-substituted polyamide. Science 254, 1497-500 (1991).
8. Nielsen, P. E. & Haaima, G. Peptide nucleic acid (PNA). A DNA mimic with a pseudopeptide backbone. Chem. Soc. Rev. 26, 73 (1997).
9. Hyrup, B. & Nielsen, P. E. Peptide Nucleic Acids (PNA): Synthesis, Properties and Potential Applications. Bioorg. Med. Chem. 4, 5-23 (1996).
10. Courtney, C. M. & Chatterjee, A. Sequence-Specific Peptide Nucleic Acid-Based Antisense Inhibitors of TEM-1 β-Lactamase and Mechanism of Adaptive Resistance. ACS Infect. Dis. 1, 253-263 (2015).
11. Good, L. & Nielsen, P. E. Inhibition of translation and bacterial growth by peptide nucleic acid targeted to ribosomal RNA. Proc. Natl. Acad. Sci. U.S.A 95, 2073-2076 (1998).
12. Mondhe, M., Chessher, A., Goh, S., Good, L. & Stach, J. E. M. Species-selective killing of bacteria by antimicrobial peptide-PNAs. PLoS One 9, e89082 (2014).
13. Bognar, A. L., Osborne, C., Shane, B., Singer, S. C. & Ferone, R. Folylpoly-γ-glutamate synthetase-dihydrofolate synthetase. J. Biol. Chem. 260, 5625-5630 (1985).
14. Phillips, G. J. & Silhavy, T. J. The E. coli ffh gene is necessary for viability and efficient protein export. Nature 359, 744-746 (1992).
15. Little, J. W. & Mount, D. W. The SOS Regulatory System of Escherichia coli. Cell 29, 11-22 (1982).
16. Das, U. & Shuman, S. 2′-Phosphate cyclase activity of RtcA: a potential rationale for the operon organization of RtcA with an RNA repair ligase RtcB in Escherichia coli and other bacterial taxa. Rna 19, 1355-1362 (2013).
17. Kilså Jensen, K., Ørum, H., Nielsen, P. E. & Nordén, B. Kinetics for Hybridization of Peptide Nucleic Acids (PNA) with DNA and RNA Studied with the BIAcore Technique.
18. Hatamoto, M., Ohashi, A. & Imachi, H. Peptide nucleic acids (PNAs) antisense effect to bacterial growth and their application potentiality in biotechnology. Appl. Microbiol. Biotechnol. 86, 397-402 (2010).
19. Kurupati, P., Tan, K. S. W., Kumarasinghe, G. & Poh, C. L. Inhibition of gene expression and growth by antisense peptide nucleic acids in a multiresistant β-lactamase-producing Klebsiella pneumoniae strain. Antimicrob. Agents Chemother. 51, 805-811 (2007).
20. Soofi, M. A. & Seleem, M. N. Targeting essential genes in Salmonella enterica serovar typhimurium with antisense peptide nucleic acid. Antimicrob. Agents Chemother. 56, 6407-6409 (2012).
21. Savard, P. & Perl, T. M. A call for action: managing the emergence of multidrug-resistant Enterobacteriaceae in the acute care settings. Curr. Opin. Infect. Dis. 25, 371-377 (2012).
22. CLSI M100 S27:2017-Performance Standards to Antimicrobial Susceptibility Testing; 27th Edition. 62-71 (2017).
23. Bérdy, J. Thoughts and facts about antibiotics: Where we are now and where we are heading. J. Antibiot. (Tokyo). 65, 441-441 (2012).
24. Hegreness, M., Shoresh, N., Damian, D., Hartl, D. & Kishony, R. Accelerated evolution of resistance in multidrug environments. Proc. Nat. Acad. Sci. U.S.A 105, 13977-81 (2008).
25. Vazquez-Torres, A. et al. Extraintestinal dissemination of Salmonella by CD18-expressing phagocytes. Nature 401, 804-808 (1999).
26. O'Leary, N. A. et al. Reference sequence (RefSeq) database at NCB: current status, taxonomic expansion, and functional annotation. Nucleic Acids Res. 44, D733-45 (2016).
27. Langmead, B., Trapnell, C., Pop, M. & Salzberg, S. L. Ultrafast and memory-efficient alignment of short DNA sequences to the human genome. Genome Biol. 10, R25 (2009).
28. Quinlan, A. R. & Hall, I. M. BEDTools: a flexible suite of utilities for comparing genomic features. Bioinformatics 26, 841-2 (2010).
29. Zhou, K. et al. Novel reference genes for quantifying transcriptional responses of Escherichia coli to protein overexpression by quantitative PCR. BMC Mol. Biol. 12, 18-26 (2011).
30. Bolger, A. M. et al. Trimmomatic: A flexible trimmer for Illumina sequence data. Bioinformatics 30, 2114-2120 (2014).
31. Zerbino, D. R. & Birney, E. Velvet: algorithms for de novo short read assembly using de Bruijn graphs. Genome Res. 18, 821-829 (2008).
32. Bankevich, A. et al. SPAdes: a new genome assembly algorithm and its applications to single-cell sequencing. J. Comput. Biol. 19, 455-477 (2012).
33. Seemann, T. Prokka: rapid prokaryotic genome annotation. Bioinformatics 30, 2068-2069 (2014).
34. Rowe, W. et al. Search Engine for Antimicrobial Resistance: A Cloud Compatible Pipeline and Web Interface for Rapidly Detecting Antimicrobial Resistance Genes Directly from Sequence Data. PLoS One 10, e0133492 (2015).
35. Gupta, S. K. et al. ARG-annot, a new bioinformatic tool to discover antibiotic resistance genes in bacterial genomes. Antimicrob. Agents Chemother. 58, 212-220 (2014).
36. Thorvaldsdóttir, H., Robinson, J. T. & Mesirov, J. P. Integrative Genomics Viewer (IGV): high-performance genomics data visualization and exploration. Brief Bioinform. 14, 178-92 (2013).
37.

SEQUENCE IDENTIFICATION
DNA
folC - E. coli
Artificial
SEQ ID NO. 1
ATACCATGATTA
DNA
rpsD - E. coli
Artificial
SEQ ID NO. 2
AGAAAATGGCAA
DNA
rpsD - K. pneumoniae
Artificial
SEQ ID NO. 3
AGAAAATGGCAA
DNA
rpsD - S. enterica
Artificial
SEQ ID NO. 4
AGAAAATGGCAA
DNA
rpsD - S. enterica, rtcA
Artificial
SEQ ID NO. 5
AGAAAATGGCAA
DNA
ffh - E. coli
Artificial
SEQ ID NO. 6
GACAATGTTTGA
DNA
ffh - K. pneumoniae
Artificial
SEQ ID NO. 7
GACAATGTTTGAT
DNA
ffh - S. enterica
Artificial
SEQ ID NO. 8
GACAATGTTTGA
DNA
lexA - E. coli
Artificial
SEQ ID NO. 9
CGGAATGAAAGC
DNA
lexA - K. pneumoniae
Artificial
SEQ ID NO. 10
CGGAATGAAAGC
DNA
lexA - S. enterica
Artificial
SEQ ID NO. 11
CGGAATGAAAGC
DNA
gyrB - E. coli
Artificial
SEQ ID NO. 12
GTTGATGTCGAA
DNA
gyrB - K. pneumoniae
Artificial
SEQ ID NO. 13
GTTGATGTCGAA
DNA
gyrB - S. enterica
Artificial
SEQ ID NO. 14
CGTTGATGTCGAA
Peptide Nucleic Acid
α-folC
Artificial
SEQ ID NO. 15
ATACCATGATTA
Peptide Nucleic Acid
α-ffh
Artificial
SEQ ID NO. 16
GACAATGTTTGA
Peptide Nucleic Acid
α-lexA
Artificial
SEQ ID NO. 17
CGGAATGAAAGC
Peptide Nucleic Acid
α-gyrB
Artificial
SEQ ID NO. 18
GTTGATGTCGAA
Peptide Nucleic Acid
α-rspD
Artificial
SEQ ID NO. 19
AGAAAATGGCAA
DNA
Antisense oligomer for α-rpsD
Artificial
SEQ ID NO. 20
ATTTAGGTGACACTATAGAAGTGGAGAAAGAAAATGGCAAGATATTTGGG
TCCTAAGCTC
DNA
Antisense oligomer for α-lexA
Artificial
SEQ ID NO. 21
ATTTAGGTGACACTATAGAAGCAGGGGGCGGAATGAAAGCGTTAACGGCC
AGGCAACAAG
DNA
Nonsense oligomer
Artificial
SEQ ID NO. 22
GAATTCGAATTCGGTCAGTGCGTCCTGCTGATGTGCTCAGTATCTCTATC
ACTGATAGGG
Amino Acid
cell penetrating peptide
Artificial
SEQ ID NO. 23
KFFKFFKFFK
DNA
gyrB forward primer
Artificial
SEQ ID NO. 24
CGGGTCCATAGTGGTTTCCC
DNA
gyrB reverse primer
Artificial
SEQ ID NO. 25
GTGAGAAACTGCGTGGCTTG
DNA
folC forward primer
Artificial
SEQ ID NO. 26
GCTCAAGCAGTTGTTCTGCC
DNA
folC reverse primer
Artificial
SEQ ID NO. 27
TCTCACCGGGCGTATGAAAG
DNA
ffh forward primer
Artificial
SEQ ID NO. 28
TTCCATACGCACCAGCACTT
DNA
ffh reverse primer
Artificial
SEQ ID NO. 29
CGCGCAGGCAGAGAAATTAG
DNA
rpsD forward primer
Artificial
SEQ ID NO. 30
CAGCCAGGTTGGCTTTTCAC
DNA
rpsD reverse primer
Artificial
SEQ ID NO. 31
AGAAGCACGTCAGCTGGTTA
DNA
lexA forward primer
Artificial
SEQ ID NO. 32
GTTAACGGCCAGGCAACAAG
DNA
lexA reverse primer
Artificial
SEQ ID NO. 33
TCAATAACGCCTTTGCGTGC
Amino Acid
holin- endolysin construct
Bacteriophage lambda
SEQ ID NO. 34
MKMPEKHDLLAAILAAKEQGIGAILAFAMAYLRGRYNGGAFTKTVIDATM
CAIIAWFIRDLLDFAGLSSNLAYITSVFIGYIGTDSIGSLIKRFAAKKAG
VEDGRNQSTGVPRYAGVVGGNRTSENQKSWLRHCRRRAIYLLRSPSQTCH
AKPKTQINRRRTLPASFPLVGCLPQAAWPERLLSEKSGRCGIAADGAWRF
TYDSWYPSGNRPLQQYLGFTAGRWLWSVRAGQPDCKIQRSGRNGQRDCMS
RVTAIISALVICIIVCLSWAVNHYRDNAITYKAQRDKNARELKLANAAIT
DMQMRQRDVAALDAKYTKELADAKAENDALRDDVAAGRRRLHIKAVCQSV
REATTASGVDNAASPRLADTAERDYFTLRERLITMQKQLEGTQKYINEQC
R
Amino Acid
Virulence regulon transcriptional
activator VirB Shigella flexneri
SEQ ID NO. 35
MVDLCNDLLSIKEGQKKEFTLHSGNKVSFIKAKIPHKRIQDLTFVNQKTN
VRDQESLTEESLADIIKTIKLQQFFPVIGREIDGRIEILDGTRRRASAIY
AGADLEVLYSKEYISTLDARKLANDIQTAKEHSIRELGIGLNFLKVSGMS
YKDIAKKENLSRAKVTRAFQAASVPQEIISLFPIASELNFNDYKILFNYY
KGLEKANESLSSTLPILKEEIKDLDTNLPPDIYKKEILNIIKKSKNRKQN
PSLKVDSLFISKDKRTYIKRKENKTNRTLIFTLSKINKTVQREIDEAIRD
IISRHLSSS

1. An antisense peptide nucleic acid configured to be complementary to at least one target sequence in a bacterial gene, wherein said peptide nucleic acid is configured to be capable of hybridizing to said target sequence and further inhibit expression of said bacterial gene; wherein said antisense peptide nucleic acid is introduced into a bacterium having an endogenous or exogenous expresses Type III secretion system configured to inject said antisense peptide nucleic acid into a eukaryotic cell; and wherein said bacterium comprises a genetically engineered bacterium having a polynucleotide coding sequence operably linked to an inducible promoter encoding a heterologous cell lysis kill switch, having at least one heterologous cell lysis protein; wherein said heterologous cell lysis protein comprises a holin-endolysin protein according to SEQ ID NO. 34.

2. (canceled)

3. The antisense peptide nucleic acid of claim 1 and further comprising a cell penetrating peptide conjugated to said antisense peptide nucleic acid sequence.

4. The antisense peptide nucleic acid of claim 3 wherein said cell penetrating peptide is selected from the group consisting of: (KFF)₃K; penetratin; NLS; TAT; Arg(9); D-Arg(9); 10HC; cyLoP-1; and Pep-1.

5. The antisense peptide nucleic acid of claim 4 wherein said cell penetrating peptide is conjugated to said antisense peptide nucleic acid sequence via a linker.

6. (canceled)

7. The antisense peptide nucleic acid of claim 1 wherein said at least one target sequence in a bacterial gene comprises a start codon of a bacterial gene.

8. The antisense peptide nucleic acid of claim 1 wherein said bacterial gene comprises a bacterial gene related to one or more antibiotic pathways such that inhibition of said bacterial gene has a bactericidal and/or bacteriostatic effect, or wherein said bacterial gene comprises a bacterial gene related to a non-traditional antibiotic pathway such that inhibition of said bacterial gene has a bactericidal and/or bacteriostatic effect.

9. (canceled)

10. The antisense peptide nucleic acid of claim 8 wherein said bacterial gene comprises a bacterial gene selected from the group consisting of: folC; ffh; lexA; gyrB; and rpsD.

11. The antisense peptide nucleic acid of claim 8 wherein said antisense peptide nucleic acid configured to be complementary to at least one target sequence in a bacterial gene comprises and antisense peptide nucleic acid configured to be complementary to a nucleic acid sequence selected from the group consisting of: SEQ ID NO's. 1-19.

12-16. (canceled)

17. The antisense peptide nucleic acid of claim 1 wherein said bacterial gene comprises a bacterial gene selected from the group consisting of: a bacterial gene in E. coli, a bacterial gene in K. pneumoniae, and/or a bacterial gene in S. enteric, a multi-drug resistant (MDR) bacteria, carbapenem resistant Enterobacteriaceae Klebsiella pneumonia (CREKP), MDR tuberculosis (MDRTB), MDR Salmonella enterica, MDR Salmonella typhimurium (MDRST), methicillin-resistant Staphylococcus aureus (MRSA), vancomycin-resistant S. aureus (VRSA), extended spectrum β-lactamase Klebsiella pneumoniae (ESBL K. pneumoniae), vancomycin-resistant Enterococcus (VRE), carbapenem-resistant Enterobacteriaceae Escherichia coli (CRE E. coli), MDR Escherichia coli (MDR E. coli), New-Delhi metallo-β-lactamase producing Klebsiella pneumoniae (NDM-1 K. pneumoniae) and MDR Acinetobacter baumannii (MRAB).

18-26. (canceled)

27. A method of delivering an antisense peptide nucleic acid to a host comprising the steps:
generating at least one antisense peptide nucleic acid configured to be complementary to at least one target sequence in a bacterial gene, wherein said antisense peptide nucleic acid is configured to be capable of hybridizing to said target sequence and inhibit expression of said bacterial gene;
transforming a delivery bacteria with a polynucleotide coding sequence operably linked to an inducible promoter encoding a heterologous cell lysis kill switch, wherein said cell lysis kill switch comprises a polynucleotide expressing:
a heterologous holin-endolysin construct according to amino acid sequence SEQ ID NO. 34;
a heterologous bacterial secretion system;
a heterologous transcriptional activator that induces expression of said heterologous bacterial secretion system;
introducing said antisense peptide nucleic acid into said transformed delivery bacteria having said heterologous cell lysis kill switch; and
introducing a therapeutically effective amount of said transformed delivery bacteria having one or more of said antisense peptide nucleic acids and further allowing the transport of said antisense peptide nucleic acid into a host cell through said bacterial secretion system and through cell membrane pores formed by said heterologous cell lysis kill switch;
lysing said transformed delivery bacteria through the action of said heterologous cell lysis kill switch.

28. The method of claim 27 wherein said bacterial secretion system comprises a bacterial secretion system selected from the group consisting of: a Type-III secretion system, and Type-IV secretion system.

29. The method of claim 27 wherein said transformed delivery bacteria is a probiotic to the host, symbiotic to the host, or endosymbiotic with the host.

30-31. (canceled)

32. The method of claim 27 wherein said heterologous transcriptional activator comprises heterologous VirB transcriptional activator according to amino acid sequence according to SEQ ID NO. 35.

33. (canceled)

34. The method of claim 27 wherein said inducible promoter comprises a promoter that is induced by entry into a host cell.

35. The method of claim 34 wherein said promoter that is induced by entry into a host cell comprises an Ipac promoter from Shigella flexneri induced by entry into a mammalian host cell.

36. The method of claim 27 and further comprising a cell penetrating peptide conjugated to said antisense peptide nucleic acid configured to be complementary to at least one target sequence in a bacterial gene and wherein said cell penetrating peptide is selected from the group consisting of: (KFF)₃K; penetratin; NLS; TAT; Arg(9); D-Arg(9); 10HC; cyLoP-1; and Pep-1.

37-38. (canceled)

39. The method of claim 27 wherein said bacterial gene comprises a bacterial gene related to one or more antibiotic pathways such that inhibition of said bacterial gene has a bactericidal and/or bacteriostatic effect or wherein said bacterial gene comprises a bacterial gene related to a non-traditional antibiotic pathway such that inhibition of said bacterial gene has a bactericidal and/or bacteriostatic effect.

40. (canceled)

41. The method of claim 39 wherein said bacterial gene comprises a bacterial gene selected from the group consisting of: folC; ffh; lexA; gyrB; and rpsD.

42. The method of claim 39 wherein said antisense peptide nucleic acid configured to be complementary to at least one target sequence in a bacterial gene comprises and antisense peptide nucleic acid configured to be complementary to a nucleic acid sequence selected from the group consisting of: SEQ ID NO's. 1-19.

43-47. (canceled)

48. The method of claim 27 wherein said bacterial gene comprises a bacterial gene selected from the group consisting of: E. coli, K. pneumoniae, S. enterica, a multi-drug resistant (MDR) bacteria, carbapenem resistant Enterobacteriaceae Klebsiella pneumonia (CREKP), MDR tuberculosis (MDRTB), MDR Salmonella enterica, MDR Salmonella typhimurium (MDRST), methicillin-resistant Staphylococcus aureus (MRSA), vancomycin-resistant S. aureus (VRSA), extended spectrum β-lactamase Klebsiella pneumoniae (ESBL K. pneumoniae), vancomycin-resistant Enterococcus (VRE), carbapenem-resistant Enterobacteriaceae Escherichia coli (CRE E. coli), MDR Escherichia coli (MDR E. coli), New-Delhi metallo-β-lactamase producing Klebsiella pneumoniae (NDM-1 K. pneumoniae) and MDR Acinetobacter baumannii (MRAB).

49-67. (canceled)
